(19) United States
(12) Patent Application Publication
(10) Pub. No.: US 2013/0150554 A1
(43) Pub. Date: Jun. 13, 2013

(54) CELL CULTURE OF GROWTH FACTOR-FREE ADAPTED CELLS

(71) Applicants: Mark Wallace Melville, Melrose, MA (US); Tara Ann Chamberlain, Chelmsford, MA (US); Martin Sinacore, Andover, MA (US)

(72) Inventors: Mark Wallace Melville, Melrose, MA (US); Tara Ann Chamberlain, Chelmsford, MA (US); Martin Sinacore, Andover, MA (US)

(73) Assignee: WYETH LLC, Madison, NJ (US)

(21) Appl. No.: 13/817,777

(22) PCT Filed: Aug. 8, 2011
(86) PCT No.: PCT/IB2011/053534

§ 371 Date: Feb. 19, 2013

Related U.S. Application Data
(60) Provisional application No. 61/375,770, filed on Aug. 20, 2010.

(51) Int. Cl. C12P 21/00

(52) U.S. Cl. CPC C12P 21/00
USPC 530/350; 435/71.1

(57) ABSTRACT

The present invention provides improved cell culture systems that allow optimum production of recombinant proteins. Among other things, the present invention provides methods of cell culture including a step of cultivating cells adapted to growth factor-free medium in a cell culture system that provides at least one growth factor.

### BACKGROUND OF THE INVENTION

Proteins have become increasingly important as diagnostic and therapeutic agents. In most cases, proteins for commercial applications are produced in cell culture, from cells that have been engineered and/or selected to produce unusually high levels of a particular protein of interest. Optimization of cell culture conditions is important for successful commercial production of proteins. Typically, to allow for an optimum growth of recombinant cells, serum or other protein supplements are added to cell culture medium to stimulate growth and help maintain growth and viability. On the other hand, many efforts have been made to decrease production cost. Because of the high costs of serum and protein supplements and a desire to minimize the use of animal-derived components and components of unknown composition, a number of protein- or serum-free medium have been developed. However, cell growth characteristics can be very different in protein- or serum-free medium as compared to serum-based medium. Therefore, there is a particular need for the development of improved cell culture systems for optimum production of proteins.

### SUMMARY OF THE INVENTION

The present invention provides improved cell culture systems for production of recombinant proteins. The present invention encompasses the unexpected discovery that cells conditioned or adapted to growth factor-free medium are more responsive to the re-addition of growth factors to the cell culture, demonstrating surprisingly superior growth and productivity, as well as reduced accumulation of free sulfhydryls, as compared to growth factor dependent culture or completely growth-factor free cell culture.

Thus, in one aspect, the present invention provides methods of cell culture including a step of cultivating cells adapted to growth factor-free medium in a cell culture system that provides at least one growth factor. In some embodiments, a method of the invention includes a step of first adapting the cells to a growth factor-free medium.

In some embodiments, the adapting step includes growing the cells in the growth factor-free medium for more than approximately 20 generations (e.g., more than 30, 40, 50, 60, 70, 80, 90, or 100 generations). In some embodiments, the adapting step includes growing the cells in the growth factor-free medium for approximately 30-300 generations. In certain embodiments, the adapting step includes growing the cells in the growth factor-free medium for approximately 25-50 generations.

In some embodiments, the growth factor-free medium is substantially free of insulin. In some embodiments, the growth factor-free medium is substantially free of growth factors. In some embodiments, the growth factor-free medium is substantially free of protein. In some embodiments, the growth factor-free medium is substantially free of insulin, peptone, hydrolysates, transferrins, and insulin-like growth factor I (IGF-I). In some embodiments, the growth factor-free medium is serum-free. In some embodiments, the growth factor-free medium is serum-free, protein-containing medium

In some embodiments, the adapting step includes first growing the cells in a medium comprising a growth factor before growing the cells in the growth factor-free medium. In some embodiments, the medium comprising the growth factor is a serum-free medium comprising the growth factor.

In some embodiments, the cell culture system is a fed batch system. In some embodiments, the fed batch system uses a base medium supplemented with one or more feed media. In some embodiments, the at least one growth factor is provided in the base medium of the fed batch system. In some embodiments, the at least one growth factor is provided in the base medium but not in a feed medium of the fed batch system. In some embodiments, the at least one growth factor is provided in a feed medium of the fed batch system. In some embodiments, the base medium and/or feed media are otherwise substantially free of other growth factors except the at least one growth factor. In some embodiments, the base medium and/or feed media are otherwise substantially free of peptone, hydrolysates, and/or transferrins except the at least one growth factor. In some embodiments, the base medium and/or feed media are otherwise substantially free of protein except the at least one growth factor. In some embodiments, the base medium and/or feed media are substantially free of serum.

In some embodiments, the at least one growth factor is selected from the group consisting of insulin, insulin-like growth factor (IGF-I), synthetic IGF-I (LR3) and combination thereof. In certain embodiments, the at least one growth factor is insulin. In some embodiments, insulin is provided at a concentration ranging from approximately 0.01 mg/L to 1 g/L. In some embodiments, the insulin is provided at a concentration of approximately 10 mg/L. In some embodiments, the insulin is provided at a concentration of approximately 2 mg/L. In some embodiments, the at least one growth factor is LR3. In some embodiments, LR3 is provided at a concentration ranging from approximately 1 ng/L to 1 mg/L (e.g., 1 ng/L to 100 μg/L). In some embodiments, LR3 is provided at a concentration of approximately 5 μg/L. In some embodiments, LR3 is provided at a concentration of approximately 50 μg/L.

In some embodiments, the cell culture system is a large-scale production system. In some embodiments, the cell culture system uses a bioreactor. In some embodiments, the cell culture system uses a shaken culture system (e.g., spin tubes, shake flasks, and large scale shaking systems).

A variety of cell types may be used in accordance with the present invention. For example, in some embodiments, the cells are mammalian cells. In some embodiments, the mammalian cells are selected from BALB/c mouse myeloma line, human retinoblasts (PER.C6), monkey kidney cells, human embryonic kidney line (293), baby hamster kidney cells (BHK), Chinese hamster ovary cells (CHO) (e.g., CHO, CHO-K1, CHO-DG44, or CHO-DUX cells), mouse sertoli cells, African green monkey kidney cells (VERO-76), human cervical carcinoma cells (HeLa), canine kidney cells, buffalo rat liver cells, human lung cells, human liver cells, mouse mammary tumor cells, TR1 cells, MRC 5 cells, FS4 cells, or human hepatoma line (Hep G2). In some embodiments, the mammalian cells are CHO cells.

In some embodiments, the cells express a recombinant protein. In some embodiments, the recombinant protein is a glycoprotein. In some embodiments, wherein the recombinant protein is selected from the group consisting of antibodies or fragments thereof, nanobodies, single domain antibodies, Small Modular ImmunoPharmaceuticals™ (SMIPs), VHH antibodies, camelid antibodies, shark single domain polypeptides (IgNAR), single domain scaffolds (e.g., fibronectin scaffolds), SCORPION™ therapeutics (single chain polypeptides comprising an N-terminal binding domain, an effector domain, and a C-terminal binding domain), growth factors, clotting factors, cytokines, fusion proteins, pharmaceutical drug substances, vaccines, enzymes and combinations thereof.

In some embodiments, a method according to the present invention further includes obtaining a recombinant protein produced by the cells. In some embodiments, a method according to the present invention further includes purifying the recombinant protein. In some embodiments, a method according to the present invention further includes preparing a pharmaceutical composition comprising the recombinant protein.

In some embodiments, the cells are cultivated under conditions such that the cell growth and/or productivity are increased as compared to control cells that are not first adapted to growth factor-free medium. In some embodiments, the cells are cultivated under conditions such that the cell growth and/or productivity are increased as compared to control cells that are cultivated in growth factor-free medium without the at least one growth factor.

In some embodiments, the cell growth is determined by viable cell density (VCD), viability, accumulated integrated viable cell density (aIVCD), biomass accumulation as measured by capacitance (ABER probe), and/or packed cell density (PCD). In some embodiments, the productivity is determined by titer, specific productivity and/or volumetric productivity. In some embodiments, the cell growth and/or productivity is increased by at least about 30% as compared to the control cells. In certain embodiments, the cell growth and/or productivity is increased by at least about 50% as compared to the control cells. In some embodiments, the titer is increased by at least 100% as compared to the control cells. In certain embodiments, the titer is increased by approximately 2- to 3-fold as compared to the control cells.

In some embodiments, the present invention provides a recombinant protein produced using inventive methods described herein.

In particular embodiments, the present invention provides methods of cell culture including steps of adapting cells to insulin-free culture and cultivating the cells in a medium that contains insulin or an insulin-like growth factor, wherein the cells are cultivated under conditions such that the cell growth and/or productivity is increased as compared to control cells that are not first adapted to insulin-free culture but cultivated under otherwise identical conditions.

In this application, the use of “or” means “and/or” unless stated otherwise. As used in this application, the term “comprise” and variations of the term, such as “comprising” and “comprises,” are not intended to exclude other additives, components, integers or steps. As used herein, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1: Exemplary insulin-free cell culture adaptation experimental design.

FIG. 2: Exemplary data demonstrating the effect of adaptation of cells producing Antibody 1 to insulin-free medium culture conditions on Qp (pg/cell/day).

FIG. 3: Exemplary data demonstrating the effect of adaptation of cells producing Nanobody 1 to insulin-free medium culture conditions on Qp (pg/cell/day).

FIG. 4: Exemplary data demonstrating the effect of adaptation of cells producing a fusion protein to insulin-free medium culture conditions on growth rate (1/hr) and percent viability.

FIG. 5: Exemplary data demonstrating the effect of adaptation of cells producing a SMIP™ to insulin-free medium culture conditions on growth rate (1/hr) and percent viability.

FIG. 6: Exemplary data demonstrating the effect of adaptation of cells producing a SMIP™ to insulin-free medium culture conditions on productivity (μg/10⁶ cells/mL) and titer (μg/mL).

FIG. 7: Exemplary data demonstrating the effect of adaptation of cells producing an antibody to insulin-free medium culture conditions on growth rate (1/hr) and percent viability.

FIG. 8: Exemplary data demonstrating viable cell density measured in insulin-free medium adapted cells (Cell Line 1) grown in fedbatch culture. Cells were transferred from insulin-free adaptation conditions at various time points (DCB, Mid 1, Mid 2, and EOS) and added to fedbatch culture.

FIG. 9: Exemplary data demonstrating the viability measured in insulin-free medium adapted cells (Cell Line 1) grown in fedbatch culture. Cells were transferred from insulin-free adaptation conditions at various time points (DCB, Mid 1, Mid 2, and EOS) and added to fedbatch culture.

FIG. 10: Exemplary data demonstrating the accumulated integrated viable cell density measured in insulin-free medium adapted cells (Cell Line 1) grown in fedbatch culture. Cells were transferred from insulin-free adaptation conditions at various time points (DCB, Mid 1, Mid 2, and EOS) and added to fedbatch culture.

FIG. 11: Exemplary data demonstrating the specific productivity (Qp; pg/cell/day) measured in insulin-free medium adapted cells (Cell Line 1) grown in fedbatch culture. Cells were transferred from insulin-free adaptation conditions at various time points (DCB, Mid 1, Mid 2, and EOS) and added to fedbatch culture.

FIG. 12: Exemplary data demonstrating titer (μg/mL) measured in insulin-free medium adapted cells (Cell Line 1) grown in fedbatch culture. Cells were transferred from insulin-free adaptation conditions at various time points (DCB, Mid 1, Mid 2, and EOS) and added to fedbatch culture.

FIG. 13: Exemplary data demonstrating the viable cell density measured in insulin-free medium adapted cells (Cell Line 2) grown in fedbatch culture. Cells were transferred from insulin-free adaptation conditions at various time points (DCB, Mid 1, Mid 2, and EOS) and added to fedbatch culture.

FIG. 14: Exemplary data demonstrating the viability measured in insulin-free medium adapted cells (Cell Line 2) grown in fedbatch culture. Cells were transferred from insulin-free adaptation conditions at various time points (DCB, Mid 1, Mid 2, and EOS) and added to fedbatch culture.

FIG. 15: Exemplary data demonstrating the accumulated integrated viable cell density measured in insulin-free medium adapted cells (Cell Line 2) grown in fedbatch culture. Cells were transferred from insulin-free adaptation conditions at various time points (DCB, Mid 1, Mid 2, and EOS) and added to fedbatch culture.

FIG. 16: Exemplary data demonstrating the titer measured in insulin-free medium adapted cells (Cell Line 2) grown in fedbatch culture. Cells were transferred from insulin-free adaptation conditions at various time points (DCB, Mid 1, Mid 2, and EOS) and added to fedbatch culture.

FIG. 17: Exemplary data demonstrating specific productivity measured in insulin-free medium adapted cells (Cell Line 2) grown in fedbatch culture. Cells were transferred from insulin-free adaptation conditions at various time points (DCB, Mid 1, Mid 2, and EOS) and added to fedbatch culture.

FIG. 18: Exemplary data demonstrating glucose utilization by insulin-free medium adapted cells (Cell Line 2) grown in fedbatch culture. Glucose concentrations (g/L) were measured in cell culture medium at various time points throughout the cell culture process.

FIG. 19: Exemplary data demonstrating lactate levels in culture medium of insulin-free medium adapted cells (Cell Line 2) grown in fedbatch culture. Lactate concentrations (g/L) were measured in cell culture medium at various time points throughout the cell culture process.

FIG. 20: Exemplary data demonstrating glutamate, glutamine, and ammonium levels in culture medium of insulin-free medium adapted cells (Cell Line 2) grown in fedbatch culture. Glutamate, glutamine, and ammonium concentrations (mmol/L) were measured in cell culture medium at various time points throughout the cell culture process.

FIG. 21: Exemplary data demonstrating sodium and potassium levels in culture medium of insulin-free medium adapted cells (Cell Line 2) grown in fedbatch culture. Sodium and potassium concentrations (mmol/L) were measured in cell culture medium at various time points throughout the cell culture process.

FIG. 22: Exemplary data demonstrating viable cell density measured in cells producing an antibody grown in various concentrations of insulin and/or LR3 in the base and/or feed media as indicated in Table 1.

FIG. 23: Exemplary data demonstrating accumulated integrated viable cell density measured in cells producing an antibody grown in various concentrations of insulin and/or LR3 in the base and/or feed media as indicated in Table 1.

FIG. 24: Exemplary data demonstrating viability measured in cells producing an antibody grown in various concentrations of insulin and/or LR3 in the base and/or feed media as indicated in Table 1.

FIG. 25: Exemplary data demonstrating residual glucose measured in cells producing an antibody grown in various concentrations of insulin and/or LR3 in the base and/or feed media as indicated in Table 1.

FIG. 26: Exemplary data lactate (g/L) measured in cells producing an antibody grown in various concentrations of insulin and/or LR3 in the base and/or feed media as indicated in Table 1.

FIG. 27: Exemplary data demonstrating titer measured in cells producing an antibody grown in various concentrations of insulin and/or LR3 in the base and/or feed media as indicated in Table 1.

FIG. 28: Exemplary data demonstrating specific productivity measured in cells producing an antibody grown in various concentrations of insulin and/or LR3 in the base and/or feed media as indicated in Table 1.

FIG. 29: Exemplary data demonstrating Ellman's signal measured in cells producing an antibody grown in various concentrations of insulin and/or LR3 in the base and/or feed media as indicated in Table 1.

FIG. 30: Exemplary data demonstrating Ellman's signal measured in cells producing an antibody grown in various concentrations of insulin and/or LR3 in the base and/or feed media as indicated in Table 1.

FIG. 31: Exemplary data demonstrating ammonium (mMol) measured in cells producing an antibody grown in various concentrations of insulin and/or LR3 in the base and/or feed media as indicated in Table 1.

FIG. 32: Exemplary data demonstrating pH measured in cells producing an antibody grown in various concentrations of insulin and/or LR3 in the base and/or feed media as indicated in Table 1.

FIG. 33: Exemplary data demonstrating titer measured in cells producing an antibody grown in various concentrations of insulin and/or LR3 in the base and/or feed media as indicated. The presence or absence (+ or −) of insulin in adaptation media is also indicated.

FIG. 34: Exemplary data demonstrating Ellman's signal measured in cells producing an antibody grown in various concentrations of insulin and/or LR3 in the base and/or feed media as indicated. The presence or absence (+ or −) of insulin in adaptation media is also indicated.

FIG. 35: Exemplary data demonstrating integrated viable cell density measured in cells producing a monoclonal antibody grown in various concentrations of insulin and/or LR3 in the base and/or feed media as indicated. The presence or absence (+ or −) of insulin in adaptation media is also indicated.

FIG. 36: Exemplary data demonstrating viability measured in cells producing a monoclonal antibody grown in various concentrations of insulin and/or LR3 in the base and/or feed media as indicated. The presence or absence (+ or −) of insulin in adaptation media is also indicated.

FIG. 37: Exemplary data demonstrating titer measured in cells producing a monoclonal antibody grown in various concentrations of insulin and/or LR3 in the base and/or feed media as indicated. The presence or absence (+ or −) of insulin in adaptation media is also indicated.

FIG. 38: Exemplary data demonstrating specific productivity (Qp; pg/cell/day) measured in cells producing a monoclonal antibody grown in various concentrations of insulin and/or LR3 in the base and/or feed media as indicated. The presence or absence (+ or −) of insulin in adaptation media is also indicated.

FIG. 39: Exemplary data demonstrating Ellman's signal measured in cells producing a monoclonal antibody grown in various concentrations of insulin and/or LR3 in the base and/or feed media as indicated. The presence or absence (+ or −) of insulin in adaptation media is also indicated.

FIG. 40: Exemplary data demonstrating titer measured in cells producing a monoclonal antibody grown in various concentrations of insulin in the base and/or feed media as indicated. The presence or absence (+ or −) of insulin in adaptation media is also indicated.

FIG. 41: Exemplary data demonstrating specific productivity (Qp; pg/cell/day) measured in cells (Cell Line 1) grown in various concentrations of insulin in the base and/or feed media as indicated.

FIG. 42: Exemplary data demonstrating titer measured in cells (Cell Line 1) grown in various concentrations of insulin in the base and/or feed media as indicated. The presence or absence (+ or −) of insulin in adaptation media is also indicated.

FIG. 43: Exemplary data demonstrating accumulated integrated viable cell density measured in cells (Cell Line 1) grown in various concentrations of insulin in the base and/or feed media as indicated. The presence or absence (+ or −) of insulin in adaptation media is also indicated.

FIG. 44: Exemplary data demonstrating titer measured in cells (Cell Line 1) grown in various concentrations of insulin in the base and/or feed media as indicated. The presence or absence (+ or −) of insulin in adaptation media is also indicated.

FIG. 45: Exemplary data demonstrating accumulated integrated viable cell density measured in cells (Cell Line 1) grown in various concentrations of insulin in the base and/or feed media as indicated. The presence or absence (+ or −) of insulin in adaptation media is also indicated.

FIG. 46: Exemplary data demonstrating specific productivity (Qp; pg/cell/day) measured in cells (Cell Line 2) grown in various concentrations of insulin in the base and/or feed media as indicated.

FIG. 47: Exemplary data demonstrating titer measured in cells (Cell Line 2) grown in various concentrations of insulin in the base and/or feed media as indicated. The presence or absence (+ or −) of insulin in adaptation media is also indicated.

FIG. 48: Exemplary data demonstrating accumulated integrated viable cell density measured in cells (Cell Line 2) grown in various concentrations of insulin in the base and/or feed media as indicated. The presence or absence (+ or −) of insulin in adaptation media is also indicated.

FIG. 49: Exemplary data demonstrating specific productivity (Qp; pg/cell/day) measured in cells (Cell Line 3) grown in various concentrations of insulin in the base and/or feed media as indicated.

FIG. 50: Exemplary data demonstrating titer measured in cells (Cell Line 3) grown in various concentrations of insulin in the base and/or feed media as indicated. The presence or absence (+ or −) of insulin in adaptation media is also indicated.

FIG. 51: Exemplary data demonstrating accumulated integrated viable cell density measured in cells (Cell Line 3) grown in various concentrations of insulin in the base and/or feed media as indicated. The presence or absence (+ or −) of insulin in adaptation media is also indicated.

FIG. 52: Exemplary data demonstrating specific productivity (Qp; pg/cell/day) measured in cells (Cell Line 4) grown in various concentrations of insulin in the base and/or feed media as indicated.

FIG. 53: Exemplary data demonstrating titer measured in cells (Cell Line 4) grown in various concentrations of insulin in the base and/or feed media as indicated. The presence or absence (+ or −) of insulin in adaptation media is also indicated.

FIG. 54: Exemplary data demonstrating accumulated integrated viable cell density measured in cells (Cell Line 4) grown in various concentrations of insulin in the base and/or feed media as indicated. The presence or absence (+ or −) of insulin in adaptation media is also indicated.

FIG. 55: Exemplary heat map result produced by analysis using Design-Expert® Software, indicating predicted desirability results in cell cultures grown in a range of insulin concentrations in the base medium (B; X axis) and feed medium (C; Y axis).

FIG. 56: Exemplary heat map result produced by analysis using Design-Expert® Software, indicating predicted titer results in cell cultures grown in a range of insulin concentrations in the base medium (B; X axis) and feed medium (C; Y axis).

### DEFINITIONS

In order for the present invention to be more readily understood, certain terms are first defined below. Additional definitions for the following terms and other terms are set forth throughout the specification.

About, Approximately: As used herein, the terms “about” and “approximately”, as applied to one or more particular cell culture conditions, refer to a range of values that are similar to the stated reference value for that culture condition or conditions. In certain embodiments, the term “about” refers to a range of values that fall within 25, 20, 19, 18, 17, 16, 15, 14, 13, 12, 11, 10, 9, 8, 7, 6, 5, 4, 3, 2, 1 percent or less of the stated reference value for that culture condition or conditions.

Adapt and Adapted: As used herein, the term “adapt”, or grammatical equivalents, when used in connection with cell culture, refers to a process of introducing cells to a particular type of cell culture condition and growing the cells for multiple generations before the end of stability. As used herein, cells or cell lines are adapted to a cell culture if the cells can grow in the cell culture for multiple generations (e.g., more than 10, 20, 30, 40, 50 generations) before the end of stability. Cells are “adapted” to a cell culture condition if the cells exhibit a growth rate and/or viability which is similar to growth rate and/or viability of the cells in a prior condition. In some embodiments, cells adapted to a culture condition exhibit a growth rate and or viability which differs from growth rate and/or viability of the cells in a prior condition by less than 20%, 10%, or 5%. In some embodiments, cells are adapted to grow in a medium lacking one or more growth factors. In some embodiments, cells are adapted to grow in medium lacking insulin. In some embodiments, cells are adapted to grow in medium lacking one or more of insulin, peptone, hydrolysates, transferrins, and IGF-1. In some embodiments, cells are adapted to grow in serum-free medium lacking insulin. “Adapting” cells to a cell culture is also referred to as “conditioning” cells to a cell culture. “Adapted” cells are also referred to as “conditioned” cells.

Amino acid: The term “amino acid” as used herein refers to any of the twenty naturally occurring amino acids that are normally used in the formation of polypeptides, or analogs or derivatives of those amino acids. Amino acids can be provided in medium to cell cultures. The amino acids provided in the medium may be provided as salts or in hydrate form.

Antibody: The term “antibody” as used herein refers to an immunoglobulin molecule or an immunologically active portion of an immunoglobulin molecule, i.e., a molecule that contains an antigen binding site which specifically binds an antigen, such as a Fab or F(ab′)₂ fragment. In certain embodiments, an antibody is a typical natural antibody known to those of ordinary skill in the art, e.g., glycoprotein comprising four polypeptide chains: two heavy chains and two light chains. In certain embodiments, an antibody is a single-chain antibody. For example, in some embodiments, a single-chain antibody comprises a variant of a typical natural antibody wherein two or more members of the heavy and/or light chains have been covalently linked, e.g., through a peptide bond. In certain embodiments, a single-chain antibody is a protein having a two-polypeptide chain structure consisting of a heavy and a light chain, which chains are stabilized, for example, by interchain peptide linkers, which protein has the ability to specifically bind an antigen. In certain embodiments, an antibody is an antibody comprised only of heavy chains such as, for example, those found naturally in members of the Camelidae family, including llamas and camels (see, for example, U.S. Pat. Nos. 6,765,087 by Casterman et al., 6,015,695 by Casterman et al., 6,005,079 and by Casterman et al., each of which is incorporated by reference in its entirety). The terms “monoclonal antibodies” and “monoclonal antibody composition”, as used herein, refer to a population of antibody molecules that contain only one species of an antigen binding site and therefore usually interact with only a single epitope or a particular antigen. Monoclonal antibody compositions thus typically display a single binding affinity for a particular epitope with which they immunoreact. The terms “polyclonal antibodies” and “polyclonal antibody composition” refer to populations of antibody molecules that contain multiple species of antigen binding sites that interact with a particular antigen.

Batch culture: The term “batch culture” as used herein refers to a method of culturing cells in which all the components that will ultimately be used in culturing the cells, including the medium (see definition of “medium” below) as well as the cells themselves, are provided at the beginning of the culturing process. A batch culture is typically stopped at some point and the cells and/or components in the medium are harvested and optionally purified.

Bioreactor: The term “bioreactor” as used herein refers to any vessel used for the growth of a mammalian cell culture. The bioreactor can be of any size so long as it is useful for the culturing of mammalian cells. Typically, the bioreactor will be at least 1 liter and may be 10, 100, 250, 500, 1000, 2500, 5000, 8000, 10,000, 12,0000 liters or more, or any volume in between. The internal conditions of the bioreactor, including, but not limited to pH and temperature, are typically controlled during the culturing period. The bioreactor can be composed of any material that is suitable for holding mammalian cell cultures suspended in medium under the culture conditions of the present invention, including glass, plastic or metal. The term “production bioreactor” as used herein refers to the final bioreactor used in the production of the polypeptide or protein of interest. The volume of the large-scale cell culture production bioreactor is typically at least 500 liters and may be 1000, 2500, 5000, 8000, 10,000, 12,0000 liters or more, or any volume in between. One of ordinary skill in the art will be aware of and will be able to choose suitable bioreactors for use in practicing the present invention.

Cell density and high cell density: The term “cell density” as used herein refers to the number of cells present in a given volume of medium. The term “high cell density” as used herein refers to a cell density that exceeds 5×10⁶/mL, 1×10⁷/mL, 5×10⁷/mL, 1×10⁸/mL, 5×10⁸/mL, 1×10⁹/mL, 5×10⁹/mL, or 1×10¹⁰/mL.

Cellular productivity: The term “cellular productivity” as used herein refers to the total amount of recombinantly expressed protein (e.g., polypeptides, antibodies, etc.) produced by a mammalian cell culture in a given amount of medium volume. Cellular productivity is typically expressed in milligrams of protein per milliliter of medium (mg/mL) or grams of protein per liter of medium (g/L).

Cell growth rate and high cell growth rate: The term “cell growth rate” as used herein refers to the rate of change in cell density expressed in “hr⁻¹” units as defined by the equation: (ln X2−ln X1)/(T2−T1) where X2 is the cell density (expressed in millions of cells per milliliter of culture volume) at time point T2 (in hours) and X1 is the cell density at an earlier time point T1. In some embodiments, the term “high cell growth rate” as used herein refers to a growth rate value that exceeds 0.023 hr⁻¹.

Cell viability: The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

Control and test: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested or monitored) is applied or present (e.g., a cell line adapted to growth factor free medium). In the second experiment, the “control,” the variable being tested is not applied or present (e.g., a control cell line that is not adapted to growth factor-free medium). In some embodiments, a control is a historical control (i.e., culture performed previously, or a result that is previously known). In some embodiments, a control is or comprises a printed or otherwise saved record. A control may be a positive control or a negative control.

Culture, Cell culture and Mammalian cell culture: These terms as used herein refer to a mammalian cell population that is grown in a medium (see definition of “medium” below) under conditions suitable to survival and/or growth of the cell population. As will be clear to those of ordinary skill in the art, these terms as used herein may refer to the combination comprising the mammalian cell population and the medium in which the population is grown.

Ellman's assays: As used herein, the term “Ellman's assays” refers to an assay performed to measure free sulfhydryl groups in cell culture medium. Ellman's reagent, 5,5′-dithio-bis-(2-nitrobenzoic acid) (DTNB), is a water-soluble compound for quantitating free sulfhydryl groups in solution. In particular, a solution of this compound produces a measurable yellow-colored product when it reacts with sulfhydryls. DTNV reacts with a free sulfhydryl groups to yield a mixed disulfide and 2-nitro-5-thiobenzoic acid (TNB). The target of DTNB in this reaction is the conjugate base (R—S—) of a free sulfhydryl group. Typically, the rate of this reaction is dependent on several factors: 1) the reaction pH, 2) the pKa′ of the sulfhydryl and 3) steric and electrostatic effects. TNB is the “colored” species produced in this reaction and has a high molar extinction coefficient in the visible range. Sulfhydryl groups may be estimated in a sample by comparison to a standard curve composed of known concentrations of a sulfhydryl-containing compound such as cysteine. Additionally or alternatively, sulfhydryl groups may be quantitated by reference to the extinction coefficient of TNB.

Fed-batch culture: The term “fed-batch culture” as used herein refers to a method of culturing cells in which additional components are provided to the culture at some time subsequent to the beginning of the culture process. The provided components typically comprise nutritional supplements for the cells which have been depleted during the culturing process. A fed-batch culture typically starts with base medium and additional components are provided as feed medium. A fed-batch culture is typically stopped at some point and the cells and/or components in the medium are harvested and optionally purified.

Feed medium: The term “feed medium” as used herein refers to a solution containing nutrients which nourish growing mammalian cells that is added after the beginning of the cell culture. A feed medium may contain components identical to those provided in the initial cell culture medium. Alternatively, a feed medium may contain one or more additional components beyond those provided in the initial cell culture medium. Additionally or alternatively, a feed medium may lack one or more components that were provided in the initial cell culture medium. In certain embodiments, one or more components of a feed medium are provided at concentrations or levels identical or similar to the concentrations or levels at which those components were provided in the initial cell culture medium. In certain embodiments, one or more components of a feed medium are provided at concentrations or levels different than the concentrations or levels at which those components were provided in the initial cell culture medium.

Functional variants: As used herein, the term “functional variants” denotes, in the context of a functional variant of an amino acid sequence (e.g., a growth factor), a molecule that retains a biological activity (e.g., activity to stimulate cell growth or proliferation) that is substantially similar to that of the original sequence. A functional variant or equivalent may be a natural derivative or is prepared synthetically. Exemplary functional variants include amino acid sequences having substitutions, deletions, or additions of one or more amino acids, provided that the biological activity of the original protein is conserved (e.g., activity to stimulate cell growth or proliferation). For example, a functional variant may have an amino acid sequence at least 70%, 75%, 80%, 85%, 90%, 95%, 96%, 97%, 98%, 99% identical to the amino acid sequence of an original protein (e.g., a growth factor such as insulin). Functional variants of insulin include naturally-occurring IGF's and synthetic variants of natural IGF's (e.g., LR3).

Gene: The term “gene” as used herein refers to any nucleotide sequence, DNA or RNA, at least some portion of which encodes a discrete final product, typically, but not limited to, a polypeptide. The term is not meant to refer only to the coding sequence that encodes the polypeptide or other discrete final product, but may also encompass regions preceding and following the coding sequence that modulate the basal level of expression (see definition of “genetic control element” below), as well as intervening sequences (“introns”) between individual coding segments (“exons”).

Genetic control element: The term “genetic control element” as used herein refers to any sequence element that modulates the expression of a gene to which it is operably linked. Genetic control elements may function by either increasing or decreasing the expression levels and may be located before, within or after the coding sequence. Genetic control elements may act at any stage of gene expression by regulating, for example, initiation, elongation or termination of transcription, mRNA splicing, mRNA editing, mRNA stability, mRNA localization within the cell, initiation, elongation or termination of translation, or any other stage of gene expression. Genetic control elements may function individually or in combination with one another.

Growth factor-free medium: The term “growth factor-free medium” as used herein encompasses any medium that is substantially free of at least one growth factor (e.g., free of at least one added cytokine, hormone (e.g., insulin), and/or other protein substance that stimulates and/or maintains cell growth or viability). For example, a growth factor-free medium may be an insulin-free medium, which is substantially free of insulin. In some embodiments, a growth factor-free medium is a medium that is substantially free of any growth factor. For example, a growth factor-free medium may be substantially free of insulin, peptone, hydrolysates, tranferrins and insulin-like growth factor I (IGF-I). In some embodiments, a growth factor-free medium is a medium that is substantially free of protein, which is also referred to as protein-free medium. Typically, a protein-free medium lacks serum or other protein supplements. The terms “medium” and “substantially” are further defined below.

Hybridoma: The term “hybridoma” as used herein refers to a cell created by fusion of an immortalized cell derived from an immunologic source and an antibody-producing cell. The resulting hybridoma is an immortalized cell that produces antibodies. The individual cells used to create the hybridoma can be from any mammalian source, including, but not limited to, rat, pig, rabbit, sheep, pig, goat, and human. The term also encompasses trioma cell lines, which result when progeny of heterohybrid myeloma fusions, which are the product of a fusion between human cells and a murine myeloma cell line, are subsequently fused with a plasma cell. Furthermore, the term is meant to include any immortalized hybrid cell line that produces antibodies such as, for example, quadromas (See, e.g., Milstein et al., Nature, 537:3053 (1983)).

Integrated Viable Cell Density: The term “integrated viable cell density” or IVCD as used herein refers to the average density of viable cells over the course of the culture multiplied by the amount of time the culture has run. In some cases, integrated viable cell density is also referred to as accumulated integrated viable cell density (aIVCD). Assuming the amount of polypeptide and/or protein produced is proportional to the number of viable cells present over the course of the culture, integrated viable cell density is a useful tool for estimating the amount of polypeptide and/or protein produced over the course of the culture.

Medium, Cell culture medium, Culture medium: These terms as used herein refer to a solution containing nutrients which nourish growing mammalian cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. The solution is preferably formulated to a pH and salt concentration optimal for cell survival and proliferation. The medium may also be a “defined medium”—a serum-free medium that contains no proteins, hydrolysates or components of unknown composition. Defined media are free of animal-derived components and all components have a known chemical structure.

Metabolic waste product: The term “metabolic waste product” as used herein refers to compounds produced by the cell culture as a result of metabolic processes that are in some way detrimental to the cell culture. Exemplary metabolic waste products include lactate, which is produced as a result of glucose metabolism, and ammonium, which is produced as a result of glutamine metabolism.

Osmolarity and Osmolality: “Osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution (1 mOsm/kg H₂O at 38° C. is equivalent to an osmotic pressure of 19 mm Hg). “Osmolarity,” by contrast, refers to the number of solute particles dissolved in 1 liter of solution. When used herein, the abbreviation “mOsm” means “milliosmoles/kg solution”.

Perfusion culture: The term “perfusion culture” as used herein refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified.

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond.

Protein: The term “protein” as used herein refers to one or more polypeptides that function as a discrete unit. If a single polypeptide is the discrete functioning unit and does require permanent physical association with other polypeptides in order to form the discrete functioning unit, the terms “polypeptide” and “protein” as used herein are used interchangeably. If discrete functional unit is comprised of more than one polypeptide that physically associate with one another, the term “protein” as used herein refers to the multiple polypeptides that are physically coupled and function together as the discrete unit.

Recombinantly expressed polypeptide and Recombinant polypeptide: These terms as used herein refer to a polypeptide expressed from a mammalian host cell that has been genetically engineered to express that polypeptide. The recombinantly expressed polypeptide can be identical or similar to polypeptides that are normally expressed in the mammalian host cell. The recombinantly expressed polypeptide can also foreign to the host cell, i.e. heterologous to peptides normally expressed in the mammalian host cell. Alternatively, the recombinantly expressed polypeptide can be chimeric in that portions of the polypeptide contain amino acid sequences that are identical or similar to polypeptides normally expressed in the mammalian host cell, while other portions are foreign to the host cell.

Seeding: The term “seeding” as used herein refers to the process of providing a cell culture to a bioreactor or another vessel. The cells may have been propagated previously in another bioreactor or vessel. Alternatively, the cells may have been frozen and thawed immediately prior to providing them to the bioreactor or vessel. The term refers to any number of cells, including a single cell.

Serum free medium: As used herein, the term “serum-free medium” refers to a medium that does not contain animal serum (usually fetal calf serum) or extracts thereof. A serum-free medium may also be a “defined medium”—a serum-free medium that contains no serum, hydrolysates or components of unknown composition. Defined media are free of animal-derived components and all components have a known chemical structure. In some embodiments provided herein, a serum free medium includes at least one growth factor (as compared to a growth factor-free medium).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Supplementary components: The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, supplementary components may be added to the initial cell culture. In certain embodiments, supplementary components may be added after the beginning of the cell culture.

Titer: The term “titer” as used herein refers to the total amount of recombinantly expressed polypeptide or protein produced by a mammalian cell culture divided by a given amount of medium volume. Titer is typically expressed in units of milligrams of polypeptide or protein per milliliter of medium.

### DETAILED DESCRIPTION

The present invention provides, among other things, improved cell culture systems for the improved production of recombinant proteins. In particular, the invention provides a method of cell culture based on cultivating cells adapted to growth factor-free medium in a cell culture system that provides at least one growth factor (e.g., insulin, IGF-I and/or LR3).

Various aspects of the invention are described in detail in the following sections. Those of ordinary skill in the art will understand, however, that various modifications to these embodiments described herein are within the scope of the appended claims. It is the claims and equivalents thereof that define the scope of the present invention, which is not and should not be limited to or by this description of certain embodiments. The use of sections is not meant to limit the invention. Each section can apply to any aspect of the invention. In this application, the use of “or” means “and/or” unless stated otherwise.

### Adaptation to Growth Factor-Free Medium

As used herein, adaptation to growth factor-free medium is a process of transitioning cells from a growth factor-containing medium to a growth factor-free medium and growing the cells under appropriate conditions such that the cells can grow in the growth factor-free medium for multiple generations (e.g., more than 10, 20, 30, 40, 50 generations) before the end of stability. Typically, adapting cells to growth factor-free medium involves growing cells over a period of time sufficient for cells to proliferate and to achieve desirable cell density, viability and/or productivity. For example, a typical adaptation process may involve growing cells in a growth factor-free medium for more than, e.g., 1, 2, 3, 4, 5, 6 weeks.

Cells may be adapted to growth factor-free medium using various processes. In general, cells may be adapted to a growth factor-free medium through, for example, many passages in the medium. According to the present invention, a growth factor-free medium may be a medium substantially free of insulin, peptone, hydrolysates, tranferrins, insulin-like growth factor I (IGF-I) and/or any other growth factor or growth factor-like components. Typically, a growth factor-free medium is substantially free of serum. In some cases, a growth factor-free medium is an entirely protein-free medium, which is substantially free of protein (also referred to as protein-free medium).

Typically, a growth factor-free medium suitable for the present invention is a chemically defined medium that provides essential and non-essential amino acids, vitamins, energy sources, lipids, and trace elements required by the cell for minimal growth and/or survival. Such a medium may also contain supplementary components that enhance growth and/or survival above the minimal rate, including, but not limited to, particular ions (such as sodium, chloride, calcium, magnesium, and phosphate), buffers, vitamins, nucleosides or nucleotides, trace elements (inorganic compounds usually present at very low final concentrations), inorganic compounds present at high final concentrations (e.g., iron), amino acids, lipids, and/or glucose or other energy source. A growth factor-free medium is preferably formulated to a pH and salt concentration optimal for cell survival and proliferation.

In some embodiments, cells may go insulin-free at the beginning of cell culture. In this case, cells are typically introduced to serum-free and growth factor-free conditions simultaneously. For example, frozen cell stocks (typically kept in a serum-free but growth factor-containing medium) may be thawed into a serum-free and insulin-free medium. In some embodiments, cells are first grown in a serum-free but growth factor-containing medium before being transitioned into growth factor-free medium. In this case, frozen cell stocks may be thawed into a serum-free but growth factor-containing medium and cultivated for a period of time (e.g., about 2 or 4 weeks) typically until the cells reach stable growth and productivities. The cells are then transitioned into a growth factor-free medium. Alternatively, cells may be first grown in serum-containing but growth factor-free medium before being transitioned into serum-free and growth factor-free medium.

Various seed densities may be used for adaptation culture. Typically, high seed densities are used to start a culture and for passages. A suitable exemplary seed density may be 0.5e6, 0.75e6, 1.0e6, 1.5e6, or 2.0e6 cells/mL. In some embodiments, seed densities may be 0.1e6, 0.2e6, 0.3e6, 0.4e6 cells/mL.

Cells may be cultured in a growth factor-free medium under standard or modified cell culture conditions. For example, cells may be grown at a temperature between approximately 25-42° C. (e.g., 25, 30, 31, 37, 40° C.). Cells may be grown in suspension or as adherent cells. Cells may also be cultured in a small volume (e.g., approximately 1 mL, 5 mL, 10 mL, 15 mL, 50 mL, or 1 L) or at a large scale (e.g., 100 L, 250 L, 400 L). Tubes, plates, flasks, bioreactors or any other containers may be used to grow cells during the adaptation process. The cell culture can be agitated or shaken to increase oxygenation of the medium and dispersion of nutrients to the cells. Typically, cell density, viability, productivity and/or titer may be measured regularly (e.g., daily, weekly or bi-weekly) to monitor the growth or productivity of a grow factor-free cell culture.

As used herein, growth factor-free adapted cells or cell lines refer to cells that can grow in a growth factor-free medium for multiple generations (e.g., more than 10, 20, 30, 40, 50, 60, 70, 80, 90, 100, 110, 120, 130 or more generations) before the end of stability. Typically a well adapted growth factor-free cell culture displays high viable cell density, viability, specific productivity, and/or titer. Growth factor-free adapted cells or cell lines are also referred to as growth factor-independent cells or cell lines.

Exemplary adaptation processes are described in detail in the Examples section (see, e.g., Example 1). Additional methods for culturing and/or adapting cells to growth factor-free medium are known in the art and can be used to practice the present invention. See, WO 97/05240, JP 2696001, U.S. Pat. No. 5,393,668, U.S. Pat. No. 6,100,061 and Burky J. E. et al., Biotechnology & Bioengineering, 2007, Vol. 96, No. 2, p 281-293, the teachings of all of which are hereby incorporated by reference.

Production Culture Systems with Re-Addition of Growth Factors

Growth factor-free adapted cells may be used for production culture. The present inventors have demonstrated that adapting or conditioning cells to a growth factor-free or protein-free medium is not only possible, but provides desirable consequences for the production culture. For example, growth factor-free adapted cells may be used in production culture also in the absence of such growth factors, displaying surprisingly superior growth and productivity as compared to growth factor-dependent cells cultured in similar conditions. More surprisingly, the inventors have discovered that the growth factor-free adapted (i.e., growth factor-independent) cells are more responsive to the re-addition of growth factors to the production culture, demonstrating significantly further enhanced growth and productivity as compared to growth-factor dependent culture or completely growth factor-free cell culture. Thus, the present invention contemplates a method of cell culture by cultivating cells adapted to growth factor-free medium in a production cell culture system that provides at least one growth factor.

Providing Growth Factors

As used herein, by providing growth factors, it is meant that one or more growth factors are added to a cell culture medium in which growth factor-free adapted cells are cultivated. As used herein, the term “growth factor” refers to any substance that is capable of stimulating cellular growth or proliferation. In some embodiments, growth factors are short peptides such as hormones. Various growth factors may be added to a production culture according to the present invention. Exemplary suitable growth factors include, but are not limited to, insulin, IGF-1, synthetic analogs of IGF-I (e.g., LR3), and functional variants thereof.

As used herein, the term “functional variants” denotes, in the context of a growth factor, a molecule that retains a biological activity (e.g., activity to stimulate cell growth or proliferation) that is substantially similar to that of the original growth factor. A functional variant or equivalent may be a natural derivative or is prepared synthetically. Exemplary functional variants include amino acid sequences having substitutions, deletions, or additions of one or more amino acids, provided that the biological activity of the original growth factor is conserved (e.g., activity to stimulate cell growth or proliferation). For example, a functional variant may have an amino acid sequence at least 50%, 55%, 60%, 65%, 70%, 75%, 80%, 85%, 90%, 95%, 96%, 97%, 98%, or 99% identical to the amino acid sequence of an original growth factor such as insulin. In some embodiments, functional variants of insulin are insulin-like growth factors. Insulin-like growth factors include, but are not limited to, IGF-1, LR3.

In some embodiments, a single growth factor (e.g., insulin) is added to a production culture. In some embodiments, a combination of growth factors may be added to a production culture. According to the present invention, growth factors may be provided at any stage during production culture. For example, growth factors may be added at the beginning of the production culture. Alternatively or additionally, growth factors may be added at one or more time points subsequently. When multiple growth factors (e.g., insulin and LR3) are used, they may be added at the same time or sequentially to a production culture.

Growth factors may be included as part of media components for production culture or added separately. For example, a growth factor may be added in the base medium, feed media, or both, of a fed batch culture. In some embodiments, a growth factor is only added in a base medium of a fed batch culture. When multiple growth factors are used, they may also be added in different media parts to a production culture. For example, one growth factor (e.g., insulin) may be added in base medium and another growth factor (e.g., LR3) may be added in feed media. Multiple growth factors may provide additive or synergistic effects in production culture. In some embodiments, growth factors may be provided prior to the production culture. For example, a growth factor (e.g., insulin) can be re-introduced into a culture (e.g., an adaptation culture or initial culture) before the cells are taken to seed a production culture.

Growth factors may be added at various concentrations. For example, a suitable concentration of an individual growth factor (or combined concentration of multiple growth factors) may range between approximately 0-2000 mg/L (e.g., 0-1000 mg/L, 0-750 mg/L, 0-500 mg/L, 0-250 mg/L, 0-200 mg/L, 0-150 mg/L, 0-100 mg/L, 0-75 mg/L, 0-50 mg/L, 0-25 mg/L, 0-10 mg/L, 0-1 mg/L, 0-750 μg/L, 0-500 μg/L, 0-250 μg/L, 0-200 μg/L, 0-150 μg/L, 1-100 μg/L, 0-75 μg/L, 0-50 μg/L, 0-40 μg/L, 0-30 μg/L, 0-25 μg/L, 0-20 μg/L, 0-15 μg/L, 0-10 μg/L, 0-5 μg/L, 0-1 μg/L, 0-750 ng/L, 0-500 ng/L, 0-250 ng/L, 0-200 ng/L, 0-150 ng/L, 0-50 ng/L, 0-25 ng/L, 0-10 ng/L, 0-5 ng/L). In some embodiments, a suitable concentration of an individual growth factor (or combined concentration of multiple growth factors) may be approximately 0.1 ng/L, 1 ng/L, 5 ng/L, 25 ng/L, 50 ng/L, 75 ng/L, 0.1 μg/L, 0.5 μg/L, 1 μg/L, 5 μg/L, 10 μg/L, 15 μg/L, 20 μg/L, 25 μg/L, 50 μg/L, 75 μg/L, 0.1 mg/L, 0.5 mg/L, 1.0 mg/L, 1.5 mg/L, 2 mg/L, 5 mg/L, 10 mg/L, 15 mg/L, 20 mg/L, 25 mg/L, 50 mg/L, 100 mg/L, 110 mg/L, 120 mg/L, 130 mg/L, 140 mg/L, 150 mg/L, 175 mg/L, 200 mg/L, 250 mg/L, 300 mg/L, 400 mg/L, 500 mg/L, 600 mg/L, 700 mg/L, 800 mg/L, 900 mg/L, 1000 mg/L, 1500 mg/L, or 2000 mg/L.

Production Cultures

Various production cultures may be used for the present invention including, but not limited to, batch cultures, fed-batch cultures, perfusion systems, and spin tube cultures. Batch culture processes typically comprise inoculating a large-scale production culture with a seed culture of a particular cell density, growing the cells under conditions conducive to cell growth and viability, harvesting the culture when the cells reach a specified cell density, and purifying the expressed protein. Fed-batch culture procedures include an additional step or steps of supplementing the batch culture with nutrients and other components that are consumed during the growth of the cells.

Media

As used herein, the term “medium” and “media” refer to a solution or solutions containing nutrients which nourish growing mammalian cells. Various media may be used for production culture including both serum-based and serum-free media. Typically, such solutions provide essential and non-essential amino acids, vitamins, energy sources, lipids, and trace elements required by the cell for minimal growth and/or survival. Such a solution may also contain supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), inorganic compounds present at high final concentrations (e.g., iron), amino acids, lipids, and/or glucose or other energy source. In certain embodiments, a medium is advantageously formulated to a pH and salt concentration optimal for cell survival and proliferation. In certain embodiments of the present invention, it may be beneficial to supplement the media with chemical inductants such as hexamethylene-bis(acetamide) (“HMBA”) and sodium butyrate (“NaB”). These optional supplements may be added at the beginning of the culture or may be added at a later point in order to replenish depleted nutrients or for another reason (e.g., as a feed medium).

A wide variety of mammalian growth media may be used in accordance with the present invention. In certain embodiments, cells may be grown in one of a variety of chemically defined media, wherein the components of the media are both known and controlled. In certain embodiments, cells may be grown in a complex medium, in which not all components of the medium are known and/or controlled.

Chemically defined growth media for mammalian cell culture have been extensively developed and published over the last several decades. All components of defined media are well characterized, and so defined media do not contain complex additives such as serum or hydrolysates. Recently, media formulations have been developed with the express purpose of supporting highly productive recombinant protein producing cell cultures and such media can be used in practicing the present invention.

In some embodiments, defined media typically includes roughly fifty chemical entities at known concentrations in water. In some embodiments, defined media require no protein components and so are referred to as protein-free defined media. Typical chemical components of the media fall into five broad categories: amino acids, vitamins, inorganic salts, trace elements, and a miscellaneous category that defies neat categorization.

Typically, trace elements refer to a variety of inorganic salts included at micromolar or lower levels. For example, commonly included trace elements are zinc, selenium, copper, and others. In some embodiments, iron (ferrous or ferric salts) can be included as a trace element in the initial cell culture medium at micromolar concentrations. Manganese is also frequently included among the trace elements as a divalent cation (MnCl₂ or MnSO₄) a range of nanomolar to micromolar concentrations. The numerous less common trace elements are usually added at nanomolar concentrations.

Not all components of complex media are well characterized, and so complex media may contain additives such as simple and/or complex carbon sources, simple and/or complex nitrogen sources, and serum, among other things. In some embodiments, complex media suitable for the present invention contains additives such as hydrolysates in addition to other components of defined medium as described herein.

Various media are known in the art and can be adapted to practice the present invention. For example, suitable exemplary media are described in U.S. Pat. Nos. 7,294,484, 7,300,773, and 7,335,491, the disclosures of all of which are hereby incorporated by reference. Various commercial media may also be used to practice the present invention.

One or more growth factors may be added to various media described herein at various concentrations according to the present invention.

Typically, serum-free media such as defined media are used for production cultures. In some embodiments, except for the re-added growth factor, suitable media for production culture are otherwise substantially free of serum, other growth factors, or typical protein supplements including peptone, hydrolysates, transferrin, etc. In some embodiments, except for the re-added growth factor, suitable media for production culture are otherwise substantially free of proteins. In some embodiments, a medium for production culture is otherwise identical to the growth factor-free medium used for adaptation except for the re-added growth factor.

Seeding

According to the present invention, cells adapted to growth factor-free medium (also known as growth factor-independent cells or cell lines) are used to start a production culture. Typically, growth factor cells suitable for production culture show good growth and viability in the growth factor-free adaptation culture. They may be taken from the adaptation culture at various stages (e.g., in the beginning, middle or near the end of an adaptation culture) to seed a production culture. The starting cell density in the production culture can be chosen by one of ordinary skill in the art. In accordance with the present invention, the starting cell density in the production culture can be as low as a single cell per culture volume. In preferred embodiments, however, starting cell densities in the production culture can range from about 2×10² viable cells per mL to about 2×10³, 2×10⁴, 1×10⁵, 2×10⁵, 1×10⁶, 2×10⁶, 5×10⁶ or 10×10⁶ viable cells per mL and higher.

In some embodiments, cells are first grown in an initial culture. The initial culture volume can be of any size, but is often smaller than the culture volume of the production bioreactor used in the final production, and frequently cells are passaged several times in bioreactors of increasing volume prior to seeding the production bioreactor.

Initial and intermediate cell cultures may be grown to any desired density before seeding the next intermediate or final production bioreactor. It is preferred that most of the cells remain alive prior to seeding, although total or near total viability is not required. In one embodiment of the present invention, the cells may be removed from the supernatant, for example, by low-speed centrifugation. It may also be desirable to wash the removed cells with a medium before seeding the next bioreactor to remove any unwanted metabolic waste products or medium components. The medium may be the medium in which the cells were previously grown or it may be a different medium or a washing solution selected by the practitioner of the present invention.

The cells may then be diluted to an appropriate density for seeding the production bioreactor. In a preferred embodiment of the present invention, the cells are diluted into the same medium that will be used in the production bioreactor. Alternatively, the cells can be diluted into another medium or solution, depending on the needs and desires of the practitioner of the present invention or to accommodate particular requirements of the cells themselves, for example, if they are to be stored for a short period of time prior to seeding the production bioreactor.

Culture Conditions

Once the production bioreactor has been seeded as described above, the cell culture is maintained in the initial growth phase under conditions conducive to the survival, growth and viability of the cell culture. The precise conditions will vary depending on the cell type, the organism from which the cell was derived, and the nature and character of the expressed recombinant protein of interest.

In accordance with the present invention, the production bioreactor can be any volume that is appropriate for large-scale production of polypeptides or proteins. In a preferred embodiment, the volume of the production bioreactor is at least 500 liters. In other preferred embodiments, the volume of the production bioreactor is 1000, 2500, 5000, 8000, 10,000, 12,000 liters or more, or any volume in between. One of ordinary skill in the art will be aware of and will be able to choose a suitable bioreactor for use in practicing the present invention. The production bioreactor may be constructed of any material that is conducive to cell growth and viability that does not interfere with expression or stability of the produced polypeptide or protein.

The temperature of the cell culture in the initial growth phase will be selected based primarily on the range of temperatures at which the cell culture remains viable. For example, during the initial growth phase, CHO cells grow well at 37° C. In general, most mammalian cells grow well within a range of about 25° C. to 42° C. Preferably, mammalian cells grow well within the range of about 35° C. to 40° C. Those of ordinary skill in the art will be able to select appropriate temperature or temperatures in which to grow cells, depending on the needs of the cells and the production requirements of the practitioner.

In one embodiment of the present invention, the temperature of the initial growth phase is maintained at a single, constant temperature. In another embodiment, the temperature of the initial growth phase is maintained within a range of temperatures. For example, the temperature may be steadily increased or decreased during the initial growth phase. Alternatively, the temperature may be increased or decreased by discrete amounts at various times during the initial growth phase. One of ordinary skill in the art will be able to determine whether a single or multiple temperatures should be used, and whether the temperature should be adjusted steadily or by discrete amounts.

The cell culture can be agitated or shaken to increase oxygenation of the medium and dispersion of nutrients to the cells. Alternatively or additionally, special sparging devices that are well known in the art can be used to increase and control oxygenation of the culture. In accordance with the present invention, one of ordinary skill in the art will understand that it can be beneficial to control or regulate certain internal conditions of the bioreactor, including but not limited to pH, temperature, oxygenation, etc.

The cells may be grown during the initial growth phase for a greater or lesser amount of time, depending on the needs of the practitioner and the requirement of the cells themselves. In one embodiment, the cells are grown for a period of time sufficient to achieve a viable cell density that is a given percentage of the maximal viable cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal viable cell density.

In another embodiment the cells are allowed to grow for a defined period of time. For example, depending on the starting concentration of the cell culture, the temperature at which the cells are grown, and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days. In some cases, the cells may be allowed to grow for a month or more. The cells would be grown for 0 days in the production bioreactor if their growth in a seed bioreactor, at the initial growth phase temperature, was sufficient that the viable cell density in the production bioreactor at the time of its inoculation is already at the desired percentage of the maximal viable cell density. The practitioner of the present invention will be able to choose the duration of the initial growth phase depending on polypeptide or protein production requirements and the needs of the cells themselves.

Shifting Culture Conditions

In accordance with the teaching of the present invention, at the end of the initial growth phase, at least one of the culture conditions may be shifted so that a second set of culture conditions is applied and a metabolic shift occurs in the culture. The accumulation of inhibitory metabolites, most notably lactate and ammonia, inhibits growth. A metabolic shift, accomplished by, e.g., a change in the temperature, pH, osmolality or chemical inductant level of the cell culture, may be characterized by a reduction in the ratio of a specific lactate production rate to a specific glucose consumption rate. In one non-limiting embodiment, the culture conditions are shifted by shifting the temperature of the culture. However, as is known in the art, shifting temperature is not the only mechanism through which an appropriate metabolic shift can be achieved. For example, such a metabolic shift can also be achieved by shifting other culture conditions including, but not limited to, pH, osmolality, and sodium butyrate levels. As discussed above, the timing of the culture shift will be determined by the practitioner of the present invention, based on polypeptide or protein production requirements or the needs of the cells themselves.

When shifting the temperature of the culture, the temperature shift may be relatively gradual. For example, it may take several hours or days to complete the temperature change. Alternatively, the temperature shift may be relatively abrupt. For example, the temperature change may be complete in less than several hours. Given the appropriate production and control equipment, such as is standard in the commercial large-scale production of polypeptides or proteins, the temperature change may even be complete within less than an hour.

The temperature of the cell culture in the subsequent growth phase will be selected based primarily on the range of temperatures at which the cell culture remains viable and expresses recombinant polypeptides or proteins at commercially adequate levels. In general, most mammalian cells remain viable and express recombinant polypeptides or proteins at commercially adequate levels within a range of about 25° C. to 42° C. Preferably, mammalian cells remain viable and express recombinant polypeptides or proteins at commercially adequate levels within a range of about 25° C. to 35° C. Those of ordinary skill in the art will be able to select appropriate temperature or temperatures in which to grow cells, depending on the needs of the cells and the production requirements of the practitioner.

In one embodiment of the present invention, the temperature of the subsequent growth phase is maintained at a single, constant temperature. In another embodiment, the temperature of the subsequent growth phase is maintained within a range of temperatures. For example, the temperature may be steadily increased or decreased during the subsequent growth phase. Alternatively, the temperature may be increased or decreased by discrete amounts at various times during the subsequent growth phase. One of ordinary skill in the art will understand that multiple discrete temperature shifts are encompassed in this embodiment. For example, the temperature may be shifted once, the cells maintained at this temperature or temperature range for a certain period of time, after which the temperature may be shifted again—either to a higher or lower temperature. The temperature of the culture after each discrete shift may be constant or may be maintained within a certain range of temperatures.

Monitoring Culture Conditions, Growth or Productivity

In certain embodiments of the present invention, the practitioner may find it beneficial to periodically monitor particular conditions of the growing cell culture. Monitoring cell culture conditions allows the practitioner to determine whether the cell growth or productivity is at optimal levels or whether the culture is about to enter into a suboptimal production phase such that the cell culture conditions may be adjusted accordingly. In order to monitor certain cell culture conditions, small aliquots of the culture are removed for analysis.

As non-limiting example, it may be beneficial to monitor temperature, pH, cell density, cell viability, integrated viable cell density, lactate levels, ammonium levels, osmolarity, cellular productivity or titer of the expressed recombinant protein. Numerous techniques are well known in the art that will allow one of ordinary skill in the art to measure these conditions. For example, cell density may be measured using a hemacytometer, a Coulter counter, or Cell density examination (CEDEX). Viable cell density may be determined by staining a culture sample with Trypan blue. Since only dead cells take up the Trypan blue, viable cell density can be determined by counting the total number of cells, dividing the number of cells that take up the dye by the total number of cells, and taking the reciprocal. HPLC can be used to determine the levels of lactate, ammonium or the expressed polypeptide or protein. Alternatively, the level of the expressed protein can be determined by standard molecular biology techniques such as coomassie staining of SDS-PAGE gels, Western blotting, Bradford assays, Lowry assays, Biuret assays, and UV absorbance. It may also be beneficial or necessary to monitor the post-translational modifications of the expressed polypeptide or protein, including phosphorylation and glycosylation.

In some embodiments, cell cultures are also monitored by Ellman's assays to detect Ellman's signals. As used herein, the term “Ellman's assays” refers to an assay performed to measure free sulfhydryl groups in cell culture medium. Ellman's reagent, 5,5′-dithio-bis-(2-nitrobenzoic acid) (DTNB), is a water-soluble compound for quantitating free sulfhydryl groups in solution. In particular, a solution of this compound produces a measurable yellow-colored product when it reacts with sulfhydryls. DTNV reacts with a free sulfhydryl groups to yield a mixed disulfide and 2-nitro-5-thiobenzoic acid (TNB). The target of DTNB in this reaction is the conjugate base (R—S—) of a free sulfhydryl group. Typically, the rate of this reaction is dependent on several factors: 1) the reaction pH, 2) the pKa′ of the sulfhydryl and 3) steric and electrostatic effects. TNB is the “colored” species produced in this reaction and has a high molar extinction coefficient in the visible range. Sulfhydryl groups may be estimated in a sample by comparison to a standard curve composed of known concentrations of a sulfhydryl-containing compound such as cysteine. Additionally or alternatively, sulfhydryl groups may be quantitated by reference to the extinction coefficient of TNB.

In some embodiments, monitoring cell culture conditions also involves comparing the cell growth, productivity, nutrition utilization and/or waste accumulation to a control. Typically, a control culture is a growth factor-dependent culture. Additionally or alternatively, a control culture is a protein or growth factor-free production culture without the re-addition of any growth factors. A proper control may be a culture that is run simultaneously to provide a comparator. Alternatively, a proper control may also be a historical control (i.e., data from a control performed previously, or historical results that are previously known). Comparison to a proper control may facilitate adjusting the cell culture conditions so that the cell growth and/or productivity may be maximized.

It is contemplated that the cells may be cultivated under cell culture conditions according to the present invention such that the cell growth and/or productivity (e.g., the cell density, cell viability, integrated viable cell density, cellular productivity and/or titer) are increased as compared to those of a growth factor-dependent culture or a protein or growth factor-free culture without the re-addition of growth factors. In some embodiments, the growth of a cell culture according to the present invention is increased by at least 10%, 20%, 30%, 40%, 50%, 60%, 70%, 80%, 90%, 100% (1-fold). The growth of a cell culture may be determined by viable cell density, viability, and/or integrated viable cell density (IVCD). In some embodiments, the productivity of a cell culture according to the present invention is increased by at least 20%, 30%, 40%, 50%, 60%, 70%, 80%, 90%, 1-fold, 1.5-fold, 2-fold, 2.5-fold, 3-fold, 3.5-fold, 4-fold, 4.5-fold or 5-fold. The productivity may be determined by specific productivity and/or titer of the expressed recombinant protein of interest.

It is also contemplated that a cell culture of the present invention has increased utilization of nutritions (e.g., glucose). In some embodiments, to maximize cell growth and production, glucose may be added back during the culture process to replenish depleted glucose. A persistent and unsolved problem with traditional growth factor-dependent culture is the production of metabolic waste products, which have detrimental effects on cell growth, viability, and production of expressed proteins. It is contemplated that a cell culture of the present invention has decreased accumulation of metabolic waste products. In particular, as described in the Examples section, a cell culture of the present invention has reduced accumulation of free sulfhydryl's as, e.g., monitored by Ellman's assays.

### Cells

Any mammalian cell or cell type susceptible to cell culture, and to expression of proteins, may be utilized in accordance with the present invention. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +/−DHFR(CHO, Urlaub and Chasin, Proc. Natl. Acad. Sci. USA, 77:4216 (1980); e.g., CHO, CHO-K1, CHO-DG44, or CHO-DUX cells); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2). In a particularly preferred embodiment, the present invention is used in the culturing of and expression of polypeptides and proteins from CHO cell lines.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and protein expression, and will be able to modify conditions as needed.

As noted above, in many instances the cells will be selected or engineered to produce high levels of protein. Often, cells are genetically engineered to produce high levels of protein, for example by introduction of a gene encoding the protein of interest and/or by introduction of control elements that regulate expression of the gene (whether endogenous or introduced) encoding the protein of interest.

Certain proteins may have detrimental effects on cell growth, cell viability or some other characteristic of the cells that ultimately limits production of the protein of interest in some way. Even amongst a population of cells of one particular type engineered to express a specific polypeptide, variability within the cellular population exists such that certain individual cells will grow better and/or produce more polypeptide of interest. In certain preferred embodiments of the present invention, the cell line is empirically selected by the practitioner for robust growth under the particular conditions chosen for culturing the cells. In particularly preferred embodiments, individual cells engineered to express a particular polypeptide are chosen for large-scale production based on cell growth, final cell density, percent cell viability, titer of the expressed polypeptide or any combination of these or any other conditions deemed important by the practitioner.

### Expression of Recombinant Proteins

Cells may be engineered to express various proteins of interest. The protein of interest may be expressed from a gene that is endogenous to the host cell, or from a gene that is introduced into the host cell through genetic engineering. The protein may be one that occurs in nature, or may alternatively have a sequence that was engineered or selected by the hand of man. An engineered protein may be assembled from other polypeptide segments that individually occur in nature, or may include one or more segments that are not naturally occurring.

Proteins that may desirably be expressed in accordance with the present invention will often be selected on the basis of an interesting biological or chemical activity. For example, the present invention may be employed to express any pharmaceutically or commercially relevant antibodies or fragments thereof, nanobodies, single domain antibodies, Small Modular ImmunoPharmaceuticals™ (SMIPs), VHH antibodies, camelid antibodies, shark single domain polypeptides (IgNAR), single domain scaffolds (e.g., fibronectin scaffolds), SCORPION™ therapeutics (single chain polypeptides comprising an N-terminal binding domain, an effector domain, and a C-terminal binding domain), growth factors, clotting factors, cytokines, fusion proteins, pharmaceutical drug substances, vaccines, enzymes, receptors and combinations thereof.

### Antibodies

Given the large number of antibodies currently in use or under investigation as pharmaceutical or other commercial agents, production of antibodies is of particular interest in accordance with the present invention. Antibodies are proteins that have the ability to specifically bind a particular antigen. Any antibody that can be expressed in a host cell may be used in accordance with the present invention. In a preferred embodiment, the antibody to be expressed is a monoclonal antibody.

In another preferred embodiment, the monoclonal antibody is a chimeric antibody. A chimeric antibody contains amino acid fragments that are derived from more than one organism. Chimeric antibody molecules can include, for example, an antigen binding domain from an antibody of a mouse, rat, or other species, with human constant regions. A variety of approaches for making chimeric antibodies have been described. See e.g., Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

In another preferred embodiment, the monoclonal antibody is a human antibody derived, e.g., through the use of ribosome-display or phage-display libraries (see, e.g., Winter et al., U.S. Pat. No. 6,291,159 and Kawasaki, U.S. Pat. No. 5,658,754) or the use of xenographic species in which the native antibody genes are inactivated and functionally replaced with human antibody genes, while leaving intact the other components of the native immune system (see, e.g., Kucherlapati et al., U.S. Pat. No. 6,657,103).

In another preferred embodiment, the monoclonal antibody is a humanized antibody. A humanized antibody is a chimeric antibody wherein the large majority of the amino acid residues are derived from human antibodies, thus minimizing any potential immune reaction when delivered to a human subject. In humanized antibodies, amino acid residues in the complementarity determining regions are replaced, at least in part, with residues from a non-human species that confer a desired antigen specificity or affinity. Such altered immunoglobulin molecules can be made by any of several techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and are preferably made according to the teachings of PCT Publication WO92/06193 or EP 0239400, all of which are incorporated herein by reference). Humanized antibodies can be commercially produced by, for example, Scotgen Limited, 2 Holly Road, Twickenham, Middlesex, Great Britain. For further reference, see Jones et al., Nature 321:522-525 (1986); Riechmann et al., Nature 332:323-329 (1988); and Presta, Curr. Op. Struct. Biol. 2:593-596 (1992), all of which are incorporated herein by reference.

In another preferred embodiment, the monoclonal, chimeric, or humanized antibodies described above may contain amino acid residues that do not naturally occur in any antibody in any species in nature. These foreign residues can be utilized, for example, to confer novel or modified specificity, affinity or effector function on the monoclonal, chimeric or humanized antibody. In another preferred embodiment, the antibodies described above may be conjugated to drugs for systemic pharmacotherapy, such as toxins, low-molecular-weight cytotoxic drugs, biological response modifiers, and radionuclides (see e.g., Kunz et al., Calicheamicin derivative-carrier conjugates, US20040082764 A1).

In one embodiment, the present invention is used to produce an antibody that specifically binds to the Aβ fragment of amyloid precursor protein or to other components of an amyloid plaque, and is useful in combating the accumulation of amyloid plaques in the brain which characterize Alzheimer's disease. (See, e.g., U.S. Provisional Application 60/636,684.) In some embodiments, the present invention is used to produce an antibody that specifically binds the HER2/neu receptor. In some embodiments, the present invention is used to produce an anti-CD20 antibody. In some embodiments, the present invention is used to produce antibodies against TNFα, CD52, CD25, VEGF, EGFR, CD11a, CD33, CD3, alpha-4 integrin, and/or IgE.

In another embodiment, antibodies of the present invention are directed against cell surface antigens expressed on target cells and/or tissues in proliferative disorders such as cancer.

In one embodiment, the antibody is an IgG1 anti-Lewis Y antibody. Lewis Y is a carbohydrate antigen with the structure Fuc [symbol] 1→2Galβ1→4[Fuc [symbol] 1→3]GlcNacβ1→3R (Abe et al. (1983) J. Biol. Chem., 258 11793-11797). Lewis Y antigen is expressed on the surface of 60% to 90% of human epithelial tumors (including those of the breast, colon, lung, and prostate), at least 40% of which overexpress this antigen, and has limited expression in normal tissues.

In order to target Ley and effectively target a tumor, an antibody with exclusive specificity to the antigen is ideally required. Thus, preferably, the anti-Lewis Y antibodies of the present invention do not cross-react with the type 1 structures (i.e., the lacto-series of blood groups (Lea and Leb)) and, preferably, do not bind other type 2 epitopes (i.e., neolacto-structure) like Lex and H-type 2 structures. An example of a preferred anti-Lewis Y antibody is designated hu3S193 (see U.S. Pat. Nos. 6,310,185; 6,518,415; 5,874,060, incorporated herein in their entirety). The humanized antibody hu3S193 (Attia, M. A., et al. 1787-1800) was generated by CDR-grafting from 3S193, which is a murine monoclonal antibody raised against adenocarcinoma cell with exceptional specificity for Ley (Kitamura, K., 12957-12961). Hu3S193 not only retains the specificity of 3S193 for Ley but has also gained in the capability to mediate complement dependent cytotoxicity (hereinafter referred to as CDC) and antibody dependent cellular cytotoxicity (hereinafter referred to as ADCC) (Attia, M. A., et al. 1787-1800). This antibody targets Ley expressing xenografts in nude mice as demonstrated by biodistribution studies with hu3S193 labeled with 125I, 111In, or 18F, as well as other radiolabels that require a chelating agent, such as 111In, 99 mTc, or 90Y (Clark, et al. 4804-4811).

In another embodiment, the antibody is one of the human anti-GDF-8 antibodies termed Myo29, Myo28, and Myo22, and antibodies and antigen-binding fragments derived therefrom. These antibodies are capable of binding mature GDF-8 with high affinity, inhibit GDF-8 activity in vitro and in vivo as demonstrated, for example, by inhibition of ActRIIB binding and reporter gene assays, and may inhibit GDF-8 activity associated with negative regulation of skeletal muscle mass and bone density. See, e.g., Veldman, et al, U.S. Patent Application No. 20040142382.

Receptors

Another class of polypeptides that have been shown to be effective as pharmaceutical and/or commercial agents includes receptors. Receptors are typically trans-membrane glycoproteins that function by recognizing an extra-cellular signaling ligand. Receptors typically have a protein kinase domain in addition to the ligand recognizing domain, which initiates a signaling pathway by phosphorylating target intracellular molecules upon binding the ligand, leading to developmental or metabolic changes within the cell. In one embodiment, the receptors of interest are modified so as to remove the transmembrane and/or intracellular domain(s), in place of which there may optionally be attached an Ig-domain. In a preferred embodiment, receptors to be produced in accordance with the present invention are receptor tyrosine kinases (RTKs). The RTK family includes receptors that are crucial for a variety of functions numerous cell types (see, e.g., Yarden and Ullrich, Ann. Rev. Biochem. 57:433-478, 1988; Ullrich and Schlessinger, Cell 61:243-254, 1990, incorporated herein by reference). Non-limiting examples of RTKs include members of the fibroblast growth factor (FGF) receptor family, members of the epidermal growth factor receptor (EGF) family, platelet derived growth factor (PDGF) receptor, tyrosine kinase with immunoglobulin and EGF homology domains-1 (TIE-1) and TIE-2 receptors (Sato et al., Nature 376(6535):70-74 (1995), incorporated herein be reference) and c-Met receptor, some of which have been suggested to promote angiogenesis, directly or indirectly (Mustonen and Alitalo, J. Cell Biol. 129:895-898, 1995). Other non-limiting examples of RTK's include fetal liver kinase 1 (FLK-1) (sometimes referred to as kinase insert domain-containing receptor (KDR) (Terman et al., Oncogene 6:1677-83, 1991) or vascular endothelial cell growth factor receptor 2 (VEGFR-2)), fins-like tyrosine kinase-1 (Flt-1) (DeVries et al. Science 255; 989-991, 1992; Shibuya et al., Oncogene 5:519-524, 1990), sometimes referred to as vascular endothelial cell growth factor receptor 1 (VEGFR-1), neuropilin-1, endoglin, endosialin, and Ax1. Those of ordinary skill in the art will be aware of other receptors that can preferably be expressed in accordance with the present invention.

In a particularly preferred embodiment, tumor necrosis factor inhibitors, in the form of tumor necrosis factor alpha and beta receptors (TNFR-1; EP 417,563 published Mar. 20, 1991; and TNFR-2, EP 417,014 published Mar. 20, 1991) are expressed in accordance with the present invention (for review, see Naismith and Sprang, J Inflamm. 47(1-2):1-7 (1995-96), incorporated herein by reference). According to one embodiment, the tumor necrosis factor inhibitor comprises a soluble TNF receptor and preferably a TNFR-Ig. In one embodiment, the preferred TNF inhibitors of the present invention are soluble forms of TNFRI and TNFRII, as well as soluble TNF binding proteins, in another embodiment, the TNFR-Ig fusion is a TNFR:Fc, a term which as used herein refers to “etanercept,” which is a dimer of two molecules of the extracellular portion of the p75 TNF-α receptor, each molecule consisting of a 235 amino acid Fc portion of human IgG1.

Growth Factors and Other Signaling Molecules

Another class of polypeptides that have been shown to be effective as pharmaceutical and/or commercial agents includes growth factors and other signaling molecules. Growth factors include glycoproteins that are secreted by cells and bind to and activate receptors on other cells, initiating a metabolic or developmental change in the receptor cell. In one embodiment, the protein of interest is an ActRIIB fusion polypeptide comprising the extracellular domain of the ActRIIB receptor and the Fc portion of an antibody (see, e.g., Wolfman, et al., ActRIIB fusion polypeptides and uses therefor, US2004/0223966 A1). In another embodiment, the growth factor may be a modified GDF-8 pro-peptide (see., e.g., Wolfman, et al., Modified and stabilized GDF propeptides and uses thereof, US2003/0104406 A1). Alternatively, the protein of interest could be a follistatin-domain-containing protein (see, e.g., Hill, et al., GASP1: a follistatin domain containing protein, US 2003/0162714 A1, Hill, et al., GASP1: a follistatin domain containing protein, US 2005/0106154 A1, Hill, et al., Follistatin domain containing proteins, US 2003/0180306 A1).

Non-limiting examples of mammalian growth factors and other signaling molecules include cytokines; epidermal growth factor (EGF); platelet-derived growth factor (PDGF); fibroblast growth factors (FGFs) such as aFGF and bFGF; transforming growth factors (TGFs) such as TGF-alpha and TGF-beta, including TGF-beta 1, TGF-beta 2, TGF-beta 3, TGF-beta 4, or TGF-beta 5; insulin-like growth factor-I and -II (IGF-I and IGF-II); des(1-3) -IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (TLs), e.g., IL-1 to IL-10; tumor necrosis factor (TNF) alpha and beta; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin, hemopoietic growth factor; enkephalinase; RANTES (regulated on activation normally T-cell expressed and secreted); human macrophage inflammatory protein (MIP-1-alpha); mullerian-inhibiting substance; relaxin A-chain; relaxin B-chain; prorelaxin; mouse gonadotropin-associated peptide; neurotrophic factors such as bone-derived neurotrophic factor (BDNF), neurotrophin-3, -4, -5, or -6 (NT-3, NT-4, NT-5, or NT-6), or a nerve growth factor such as NGF-beta. One of ordinary skill in the art will be aware of other growth factors or signaling molecules that can be expressed in accordance with the present invention.

G-Protein Coupled Receptors

Another class of polypeptides that have been shown to be effective as pharmaceutical and/or commercial agents includes growth factors and other signaling molecules. G-protein coupled receptors (GPCRs) are proteins that have seven transmembrane domains. Upon binding of a ligand to a GPCR, a signal is transduced within the cell which results in a change in a biological or physiological property of the cell.

GPCRs, along with G-proteins and effectors (intracellular enzymes and channels which are modulated by G-proteins), are the components of a modular signaling system that connects the state of intracellular second messengers to extracellular inputs. These genes and gene-products are potential causative agents of disease.

Specific defects in the rhodopsin gene and the V2 vasopressin receptor gene have been shown to cause various forms of autosomal dominant and autosomal recessive retinitis pigmentosa, nephrogenic diabetes insipidus. These receptors are of critical importance to both the central nervous system and peripheral physiological processes. The GPCR protein superfamily now contains over 250 types of paralogues, receptors that represent variants generated by gene duplications (or other processes), as opposed to orthologues, the same receptor from different species. The superfamily can be broken down into five families: Family I, receptors typified by rhodopsin and the beta2-adrenergic receptor and currently represented by over 200 unique members; Family II, the recently characterized parathyroid hormone/calcitonin/secretin receptor family; Family III, the metabotropic glutamate receptor family in mammals; Family IV, the cAMP receptor family, important in the chemotaxis and development of D. discoideum; and Family V, the fungal mating pheromone receptors such as STE2.

GPCRs include receptors for biogenic amines, for lipid mediators of inflammation, peptide hormones, and sensory signal mediators. The GPCR becomes activated when the receptor binds its extracellular ligand. Conformational changes in the GPCR, which result from the ligand-receptor interaction, affect the binding affinity of a G protein to the GPCR intracellular domains. This enables GTP to bind with enhanced affinity to the G protein.

Activation of the G protein by GTP leads to the interaction of the G protein α subunit with adenylate cyclase or other second messenger molecule generators. This interaction regulates the activity of adenylate cyclase and hence production of a second messenger molecule, cAMP. cAMP regulates phosphorylation and activation of other intracellular proteins. Alternatively, cellular levels of other second messenger molecules, such as cGMP or eicosinoids, may be upregulated or downregulated by the activity of GPCRs. The G protein a subunit is deactivated by hydrolysis of the GTP by GTPase, and the α, β, and γ subunits reassociate. The heterotrimeric G protein then dissociates from the adenylate cyclase or other second messenger molecule generator. Activity of GPCR may also be regulated by phosphorylation of the intra- and extracellular domains or loops.

Glutamate receptors form a group of GPCRs that are important in neurotransmission. Glutamate is the major neurotransmitter in the CNS and is believed to have important roles in neuronal plasticity, cognition, memory, learning and some neurological disorders such as epilepsy, stroke, and neurodegeneration (Watson, S. and S. Arkinstall (1994) The G-Protein Linked Receptor Facts Book, Academic Press, San Diego Calif., pp. 130-132). These effects of glutamate are mediated by two distinct classes of receptors termed ionotropic and metabotropic. Ionotropic receptors contain an intrinsic cation channel and mediate fast excitatory actions of glutamate. Metabotropic receptors are modulatory, increasing the membrane excitability of neurons by inhibiting calcium dependent potassium conductances and both inhibiting and potentiating excitatory transmission of ionotropic receptors. Metabotropic receptors are classified into five subtypes based on agonist pharmacology and signal transduction pathways and are widely distributed in brain tissues.

The vasoactive intestinal polypeptide (VIP) family is a group of related polypeptides whose actions are also mediated by GPCRs. Key members of this family are VIP itself, secretin, and growth hormone releasing factor (GRF). VIP has a wide profile of physiological actions including relaxation of smooth muscles, stimulation or inhibition of secretion in various tissues, modulation of various immune cell activities. and various excitatory and inhibitory activities in the CNS. Secretin stimulates secretion of enzymes and ions in the pancreas and intestine and is also present in small amounts in the brain. GRF is an important neuroendocrine agent regulating synthesis and release of growth hormone from the anterior pituitary (Watson, S. and S. Arkinstall supra, pp. 278-283).

Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995), the contents of which is incorporated herein by reference.

GPCRs are a major target for drug action and development. In fact, receptors have led to more than half of the currently known drugs (Drews, Nature Biotechnology, 1996, 14: 1516) and GPCRs represent the most important target for therapeutic intervention with 30% of clinically prescribed drugs either antagonizing or agonizing a GPCR (Milligan, G. and Rees, S., (1999) TIPS, 20: 118-124). This demonstrates that these receptors have an established, proven history as therapeutic targets.

In general, practitioners of the present invention will selected their polypeptide of interest, and will know its precise amino acid sequence. Any given protein that is to be expressed in accordance with the present invention will have its own idiosyncratic characteristics and may influence the cell density or viability of the cultured cells, and may be expressed at lower levels than another polypeptide or protein grown under identical culture conditions. One of ordinary skill in the art will be able to appropriately modify the steps and compositions of the present invention in order to optimize cell growth and/or production of any given expressed polypeptide or protein.

Enzymes

Another class of proteins that have been shown to be effective as pharmaceutical and/or commercial agents includes enzymes. Enzymes may be proteins whose enzymatic activity may be affected by cell culture conditions under which they were produced. Thus, production of enzymes with desirable enzymatic activity in accordance with the present invention is also of particular interest. One of ordinary skill in the art will be aware of many known enzymes that may be expressed by cells in culture.

Non-limiting examples of enzymes include a carbohydrase, such as an amylase, a cellulase, a dextranase, a glucosidase, a galactosidase, a glucoamylase, a hemicellulase, a pentosanase, a xylanase, an invertase, a lactase, a naringanase, a pectinase and a pullulanase; a protease such as an acid protease, an alkali protease, bromelain, ficin, a neutral protease, papain, pepsin, a peptidase (e.g., an aminopeptidase and carboxypeptidase), rennet, rennin, chymosin, subtilisin, thermolysin, an aspartic proteinase, and trypsin; a lipase or esterase, such as a triglyceridase, a phospholipase, a pregastric esterase, a phosphatase, a phytase, an amidase, an iminoacylase, a glutaminase, a lysozyme, and a penicillin acylase; an isomerase such as glucose isomerase; an oxidoreductases, such as an amino acid oxidase, a catalase, a chloroperoxidase, a glucose oxidase, a hydroxysteroid dehydrogenase or a peroxidase; a lyase such as a acetolactate decarboxylase, an aspartic decarboxylase, a fumarase or a histadase; a transferase such as cyclodextrin glycosyltranferase; a ligase; a chitinase, a cutinase, a deoxyribonuclease, a laccase, a mannosidase, a mutanase, a pectinolytic enzyme, a polyphenoloxidase, ribonuclease and transglutaminase.

Genetic Control Elements

As will be clear to those of ordinary skill in the art, genetic control elements may be employed to regulate gene expression of the polypeptide or protein. Such genetic control elements should be selected to be active in the relevant host cell. Control elements may be constitutively active or may be inducible under defined circumstances. Inducible control elements are particularly useful when the expressed protein is toxic or has otherwise deleterious effects on cell growth and/or viability. In such instances, regulating expression of the polypeptide or protein through inducible control elements may improve cell viability, cell density, and/or total yield of the expressed polypeptide or protein. A large number of control elements useful in the practice of the present invention are known and available in the art.

Representative constitutive mammalian promoters that may be used in accordance with the present invention include, but are not limited to, the hypoxanthine phosphoribosyl transferase (HPTR) promoter, the adenosine deaminase promoter, the pyruvate kinase promoter, the beta-actin promoter as well as other constitutive promoters known to those of ordinary skill in the art. Additionally, viral promoters that have been shown to drive constitutive expression of coding sequences in eukaryotic cells include, for example, simian virus promoters, herpes simplex virus promoters, papilloma virus promoters, adenovirus promoters, human immunodeficiency virus (HIV) promoters, Rous sarcoma virus promoters, cytomegalovirus (CMV) promoters, the long terminal repeats (LTRs) of Moloney murine leukemia virus and other retroviruses, the thymidine kinase promoter of herpes simplex virus as well as other viral promoters known to those of ordinary skill in the art.

Inducible promoters drive expression of operably linked coding sequences in the presence of an inducing agent and may also be used in accordance with the present invention. For example, in mammalian cells, the metallothionein promoter is induces transcription of downstream coding sequences in the presence of certain metal ions. Other inducible promoters will be recognized by and/or known to those of ordinary skill in the art.

In general, the gene expression sequence will also include 5′ non-transcribing and 5′ non-translating sequences involved with the initiation of transcription and translation, respectively, such as a TATA box, capping sequence, CAAT sequence, and the like. Enhancer elements can optionally be used to increase expression levels of the polypeptides or proteins to be expressed. Examples of enhancer elements that have been shown to function in mammalian cells include the SV40 early gene enhancer, as described in Dijkema et al., EMBO J. (1985) 4: 761 and the enhancer/promoter derived from the long terminal repeat (LTR) of the Rous Sarcoma Virus (RSV), as described in Gorman et al., Proc. Natl. Acad. Sci. USA (1982b) 79:6777 and human cytomegalovirus, as described in Boshart et al., Cell (1985) 41:521.

Systems for linking control elements to coding sequences are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N.Y., 1989, which is incorporated herein by reference). Commercial vectors suitable for inserting preferred coding sequence for expression in various mammalian cells under a variety of growth and induction conditions are also well known in the art.

Introduction of Coding Sequences and Related Control Elements into Host Cells

Methods suitable for introducing into mammalian host cells nucleic acids sufficient to achieve expression of the proteins of interest are well known in the art. See, for example, Gething et al., Nature, 293:620-625 (1981); Mantei et al., Nature, 281:40-46 (1979); Levinson et al.; EP 117,060; and EP 117,058, all incorporated herein by reference.

For mammalian cells, preferred methods of transformation include the calcium phosphate precipitation method of Graham and van der Erb, Virology, 52:456-457 (1978) or the Lipofectamine™. (Gibco BRL) Method of Hawley-Nelson, Focus 15:73 (1193). General aspects of mammalian cell host system transformations have been described by Axel in U.S. Pat. No. 4,399,216 issued Aug. 16, 1983. For various techniques for transforming mammalian cells, see Keown et al., Methods in Enzymology (1989), Keown et al., Methods in Enzymology, 185:527-537 (1990), and Mansour et al., Nature, 336:348-352 (1988). Non-limiting representative examples of suitable vectors for expression of polypeptides or proteins in mammalian cells include pcDNA1; pCD, see Okayama, et al. (1985) Mol. Cell Biol. 5:1136-1142; pMClneo Poly-A, see Thomas, et al. (1987) Cell 51:503-512; and a baculovirus vector such as pAC 373 or pAC 610.

In preferred embodiments, the polypeptide or protein is stably transfected into the host cell. However, one of ordinary skill in the art will recognize that the present invention can be used with either transiently or stably transfected mammalian cells.

### Isolation of Expressed Protein

In general, it will typically be desirable to isolate and/or purify proteins or polypeptides expressed according to the present invention. In a preferred embodiment, the expressed polypeptide or protein is secreted into the medium and thus cells and other solids may be removed, as by centrifugation or filtering for example, as a first step in the purification process. This embodiment is particularly useful when used in accordance with the present invention, since the methods and compositions described herein result in increased cell viability. As a result, fewer cells die during the culture process, and fewer proteolytic enzymes are released into the medium which can potentially decrease the yield of the expressed polypeptide or protein.

Alternatively, the expressed polypeptide or protein is bound to the surface of the host cell. In this embodiment, the media is removed and the host cells expressing the polypeptide or protein are lysed as a first step in the purification process. Lysis of mammalian host cells can be achieved by any number of means well known to those of ordinary skill in the art, including physical disruption by glass beads and exposure to high pH conditions.

The polypeptide or protein may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly desired when cells must be lysed in order to isolate and purify the expressed polypeptide or protein. One of ordinary skill in the art will appreciate that the exact purification technique will vary depending on the character of the polypeptide or protein to be purified, the character of the cells from which the polypeptide or protein is expressed, and the composition of the medium in which the cells were grown.

### Pharmaceutical Formulations

In certain preferred embodiments of the invention, produced polypeptides or proteins will have pharmacologic activity and will be useful in the preparation of pharmaceuticals. Inventive compositions as described above may be administered to a subject or may first be formulated for delivery by any available route including, but not limited to parenteral (e.g., intravenous), intradermal, subcutaneous, oral, nasal, bronchial, opthalmic, transdermal (topical), transmucosal, rectal, and vaginal routes. Inventive pharmaceutical compositions typically include a purified polypeptide or protein expressed from a mammalian cell line, a delivery agent (i.e., a cationic polymer, peptide molecular transporter, surfactant, etc., as described above) in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the purified polypeptide or protein in the required amount in an appropriate solvent with one or a combination of ingredients enumerated above, as required, followed by filtered sterilization. Generally, dispersions are prepared by incorporating the purified polypeptide or protein expressed from a mammalian cell line into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the purified polypeptide or protein can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions comprising a purified polypeptide or protein expressed from a mammalian cell line and a delivery agent are preferably delivered in the form of an aerosol spray from a pressured container or dispenser which contains a suitable propellant, e.g., a gas such as carbon dioxide, or a nebulizer. The present invention particularly contemplates delivery of the compositions using a nasal spray, inhaler, or other direct delivery to the upper and/or lower airway. Intranasal administration of DNA vaccines directed against influenza viruses has been shown to induce CD8 T cell responses, indicating that at least some cells in the respiratory tract can take up DNA when delivered by this route, and the delivery agents of the invention will enhance cellular uptake. According to certain embodiments of the invention the compositions comprising a purified polypeptide expressed from a mammalian cell line and a delivery agent are formulated as large porous particles for aerosol administration.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

The compositions can also be prepared in the form of suppositories (e.g., with conventional suppository bases such as cocoa butter and other glycerides) or retention enemas for rectal delivery.

In one embodiment, the compositions are prepared with carriers that will protect the polypeptide or protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active polypeptide or protein calculated to produce the desired therapeutic effect in association with the required pharmaceutical carrier.

The polypeptide or protein expressed according to the present invention can be administered at various intervals and over different periods of time as required, e.g., one time per week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a polypeptide or protein as described herein can include a single treatment or, in many cases, can include a series of treatments. It is furthermore understood that appropriate doses may depend upon the potency of the polypeptide or protein and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

The present invention includes the use of inventive compositions for treatment of nonhuman animals. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

Inventive pharmaceutical compositions can be included in a container, pack, or dispenser together with instructions for administration.

The foregoing description is to be understood as being representative only and is not intended to be limiting. Alternative methods and materials for implementing the invention and also additional applications will be apparent to one of skill in the art, and are intended to be included within the accompanying claims.

### EXAMPLES

### Example 1

### Adaptation to Insulin-Free Medium

This example demonstrates that various cell lines may be adapted to insulin-free medium through, for example, many passages in medium substantially lacking insulin, other growth factors and/or any protein components. In some cases, cells may go insulin-free at the start of adaptation culture. In some embodiments, cell lines can be transitioned well into serum-free and insulin-free simultaneously. In some cases, it may be desirable by first growing cells in serum-free but insulin-containing medium before transitioning into insulin-free medium. Similar methods may be used to adapt cells to other growth factor-free media.

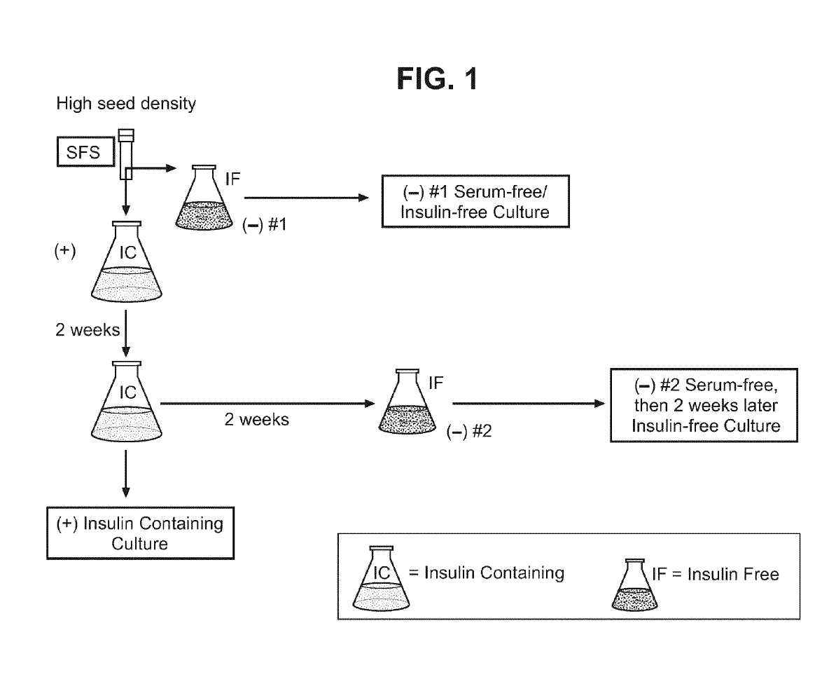

An exemplary adaptation experimental design is illustrated in FIG. 1. Specifically, frozen cell stocks may be thawed directly into serum-free medium/insulin-free culture (adaptation process #1). Alternatively, cells may first be grown in serum-free but insulin-containing culture for 2 weeks and then subsequently transitioned to insulin-free culture (adaptation process #2). Cells grown in insulin-containing culture are used as control. Typically, high seed density is used for passages in the adaptation process.

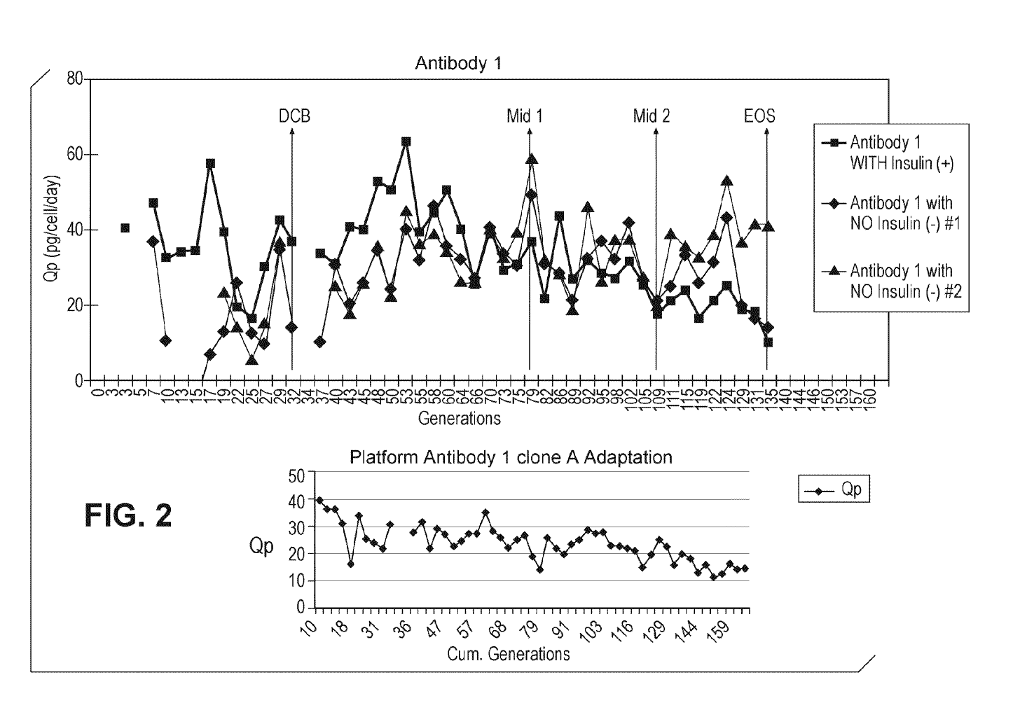

As shown in FIG. 2, a clonal cell line producing Antibody 1 was adapted using adaptation process #1 or #2. Control cells were grown in insulin-containing culture. All three cell cultures started from thaw at about 32 generations and grown for about 135 generations before reaching the end of stability (EOS). Cellular productivity (Qp; pg/cell/day) was measured at intervals throughout the cell culture process and is depicted in FIG. 2. Both insulin-free cultures had similar and stable growth and productivity levels. Control cells demonstrated signs of instability during the culture process.

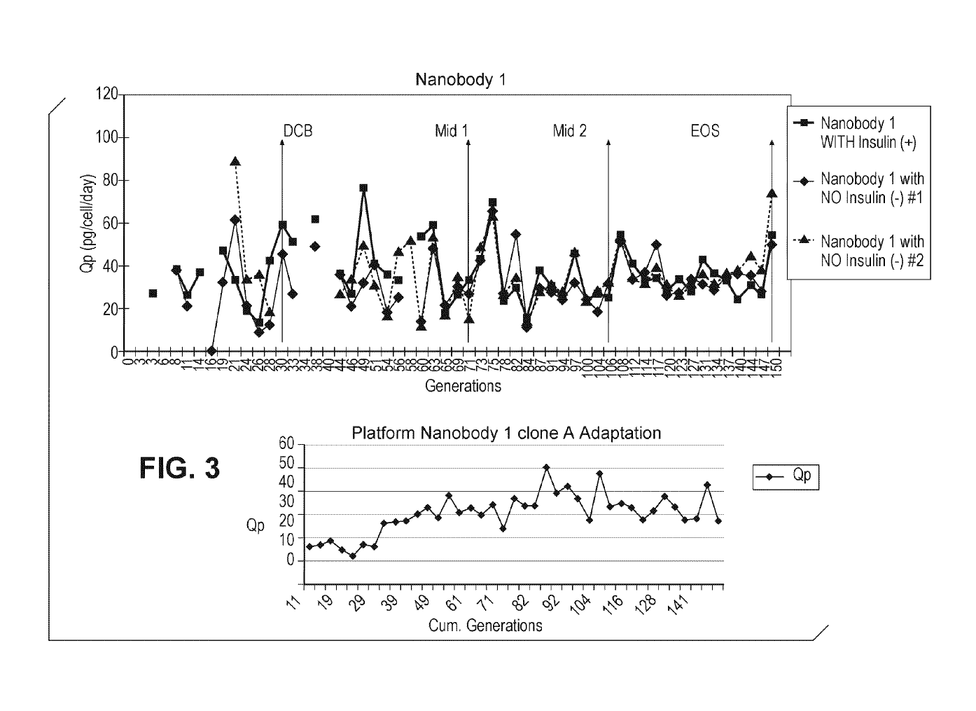

Another example is shown in FIG. 3. A clonal cell line producing Nanobody 1 was adapted using adaptation process #1 or #2. Control cells were grown in insulin-containing culture. All three cultures started from thaw at about 32 generations and were grown for about 150 generations before EOS. Cellular productivity (Qp; pg/cell/day) was measured at intervals throughout the cell culture process and is depicted in FIG. 3. No major differences were observed in cultures with or without insulin. Cells were clumpy and chunky at various times. In general, the cell lines transitioned well into insulin-free medium.

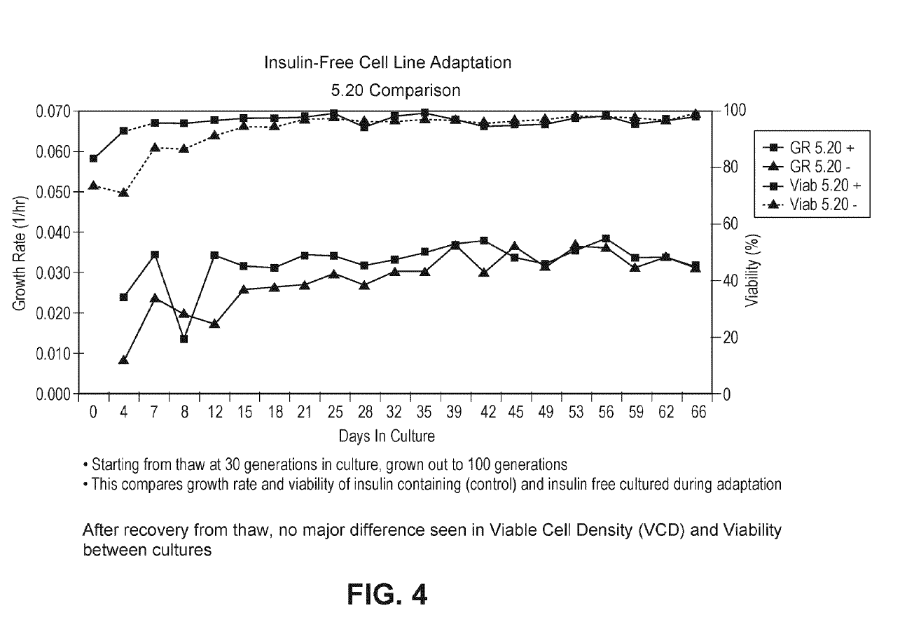

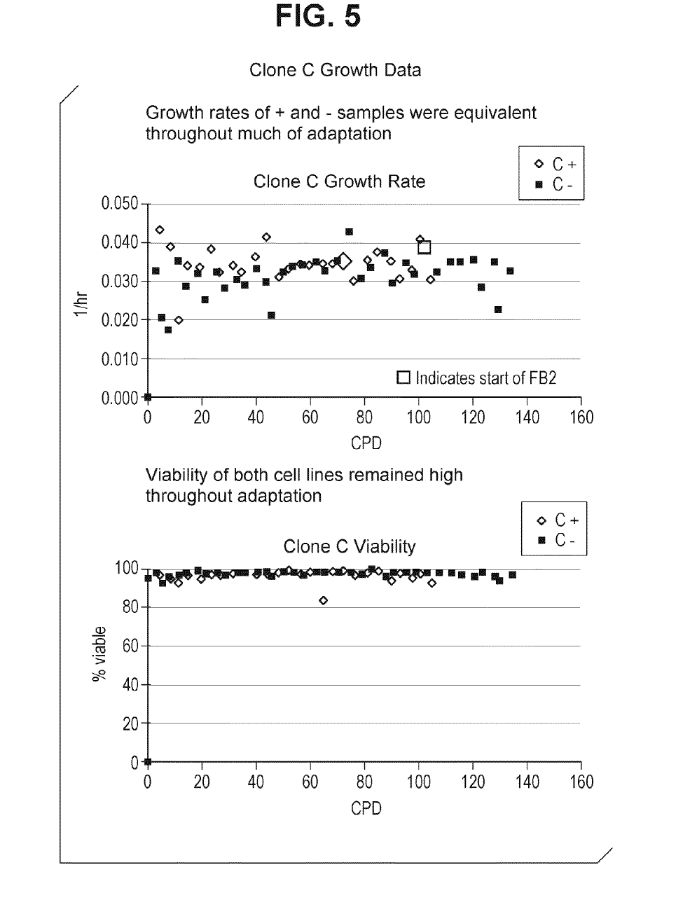

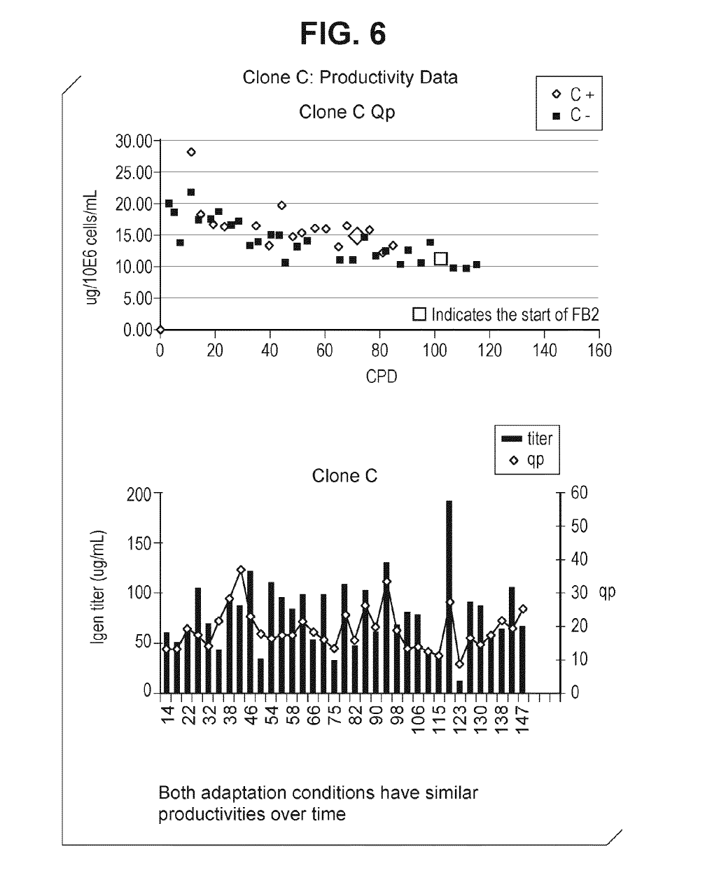

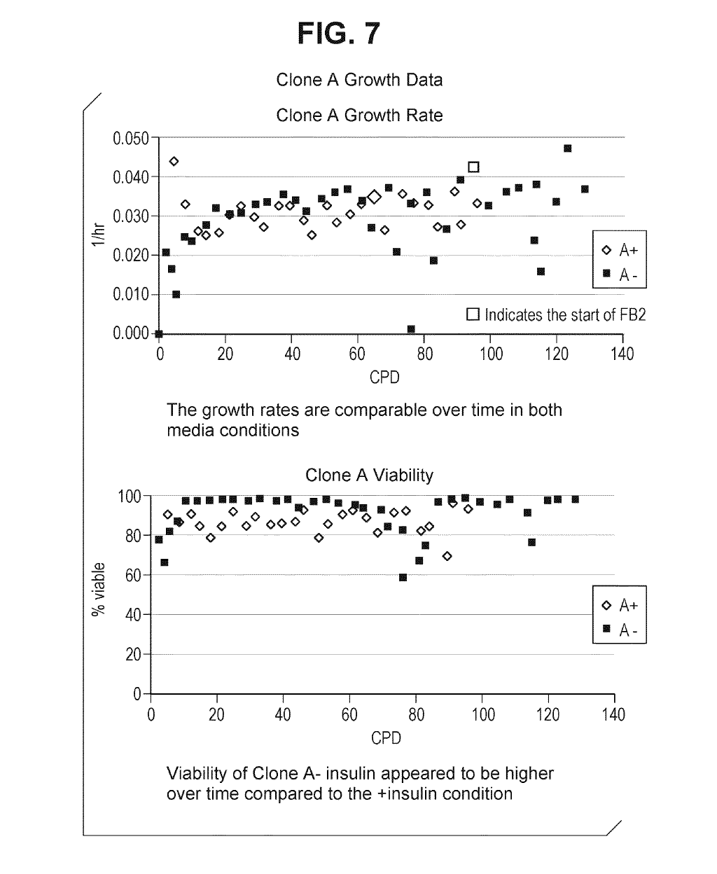

Additional exemplary adaptation results are shown in FIGS. 4-7.

All of these results demonstrate that various cell lines may be successfully adapted to grow in insulin-free culture with high viability, growth rate and productivity. So far more than 10 cell lines expressing antibodies, fusion proteins and nanobodies have been successfully adapted to insulin-free culture.

### Example 2

### Fed Batch Production Cultures with Re-Addition of Growth Factors

Many insulin-free adapted cells have been tested in fed batch culture with little to no negative impact on growth, viability or productivity. Experiments described in this example showed that, surprisingly, cells conditioned or adapted to growth factor-free medium are more responsive to the re-addition of growth factors in the production culture, demonstrating even further enhanced growth and productivity, as compared to growth factor-dependent culture, or completely growth factor-free cell culture.

Experiment 1

Cells adapted well may be used to run fed batch or other type of production culture. Typically, decision point is every two weeks. When cells show good growth and viability, they may be taken from adaptation culture and put into fed batch. In this experiment, insulin-free adapted cells and control cells were taken at DCB (Development Cell Bank), Mid 1 (Middle of Culture Timepoint 1), Mid 2 (Middle of Culture Timepoint 2), and EOS (End of Stability of Culture Timepoint) from the adaptation culture to run a fed batch culture. In this experiment, base medium of the fed batch contained Medium A basal medium supplemented with amino acids and insulin at 10 mg/L. Medium B containing 140 mg/L insulin was used as feed media. Cells were grown in 15 mL culture volume. 1.5e6 cells/mL seed density was used. pH adjusted post-temperature shift at day 7 and 9. Supplemental feed at day 4 (5%), day 7 (4%) and day 9 (3%). Two cell lines (Cell Line 1 expressing a nanobody and Cell Line 2 expressing a monoclonal antibody) were used in this experiment.

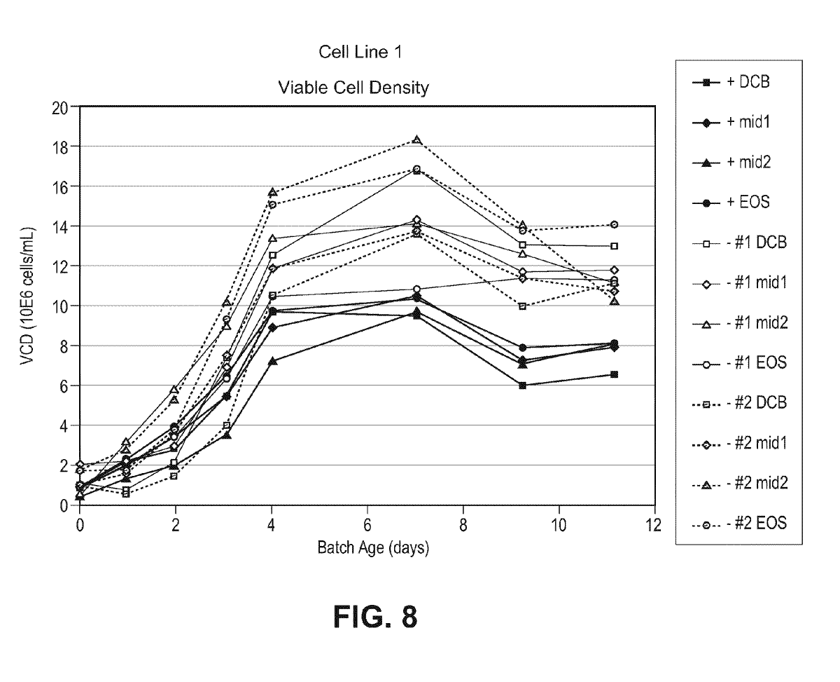

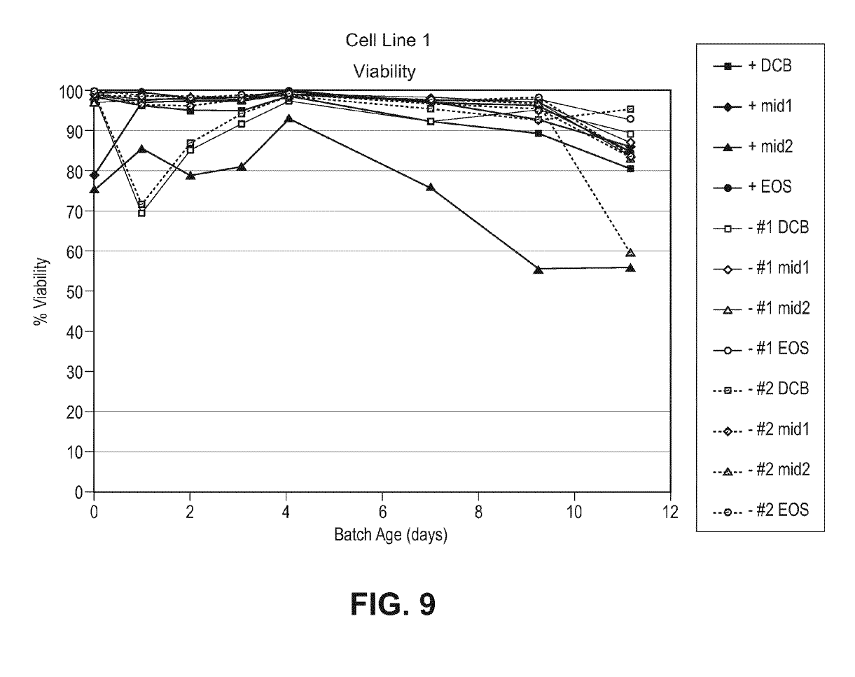

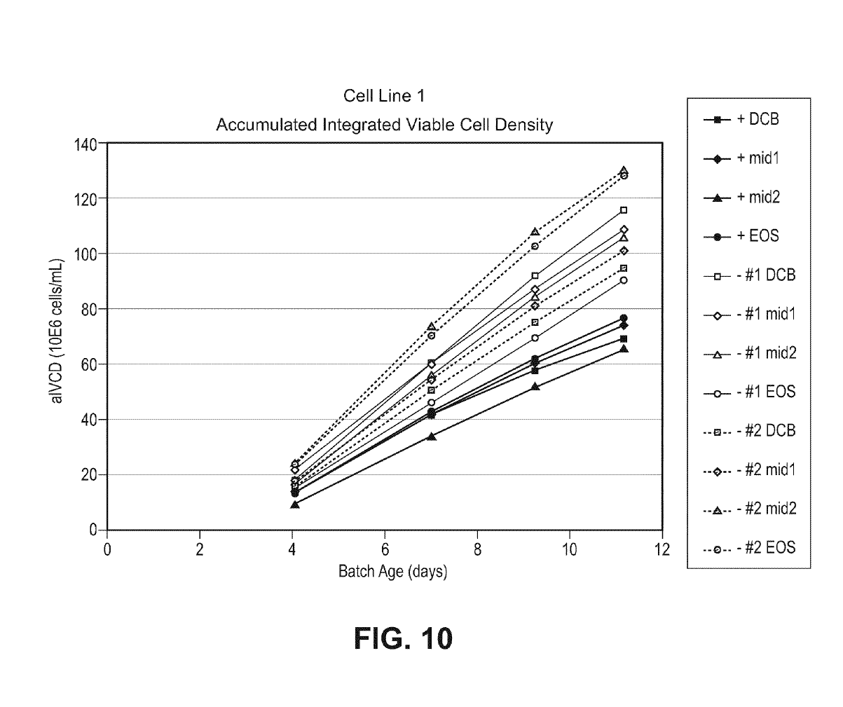

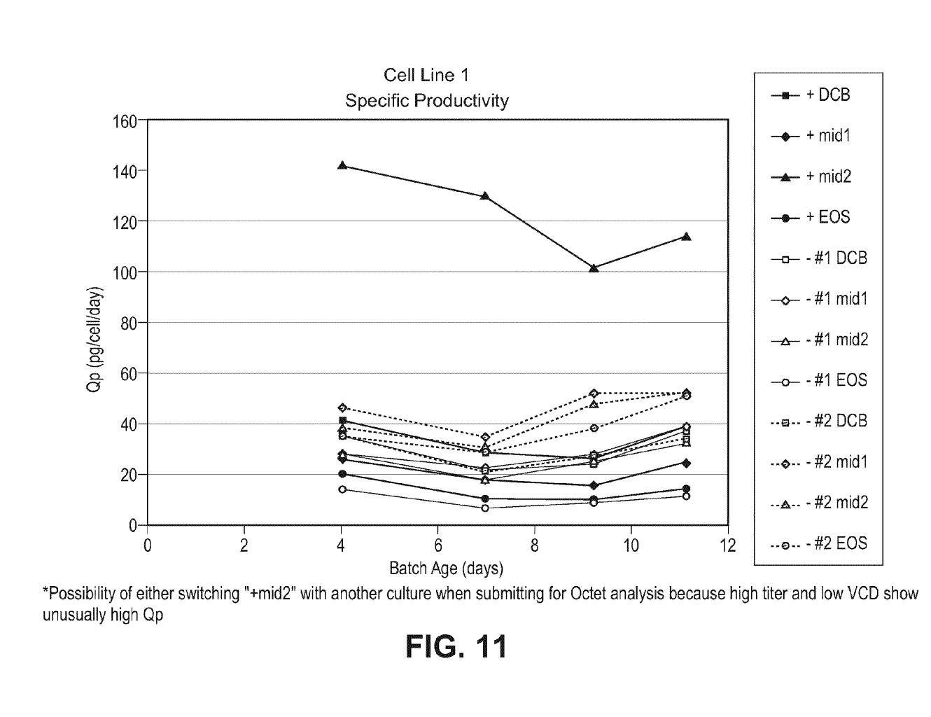

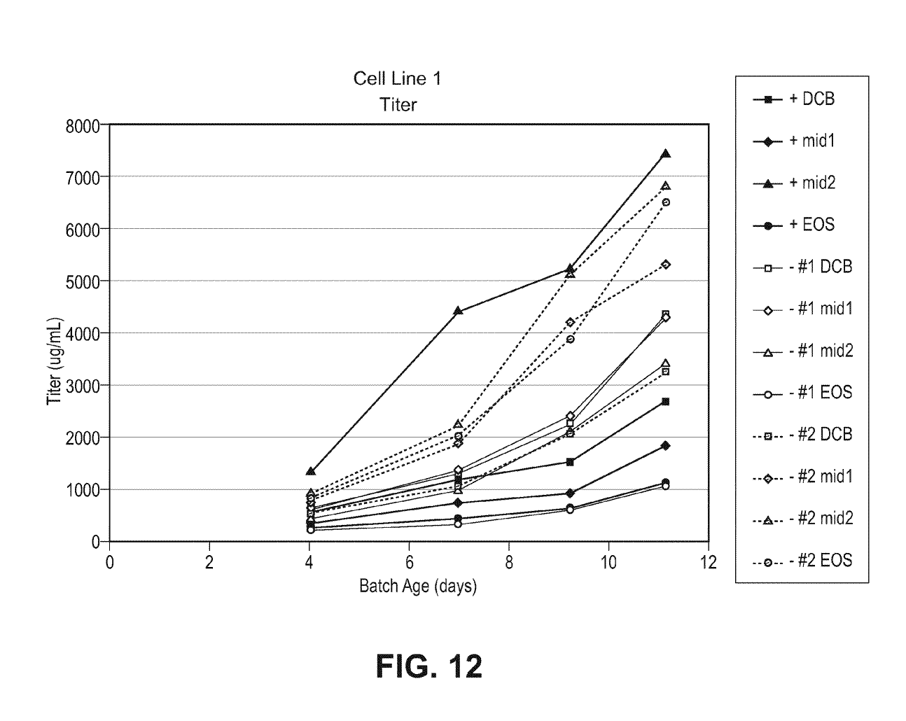

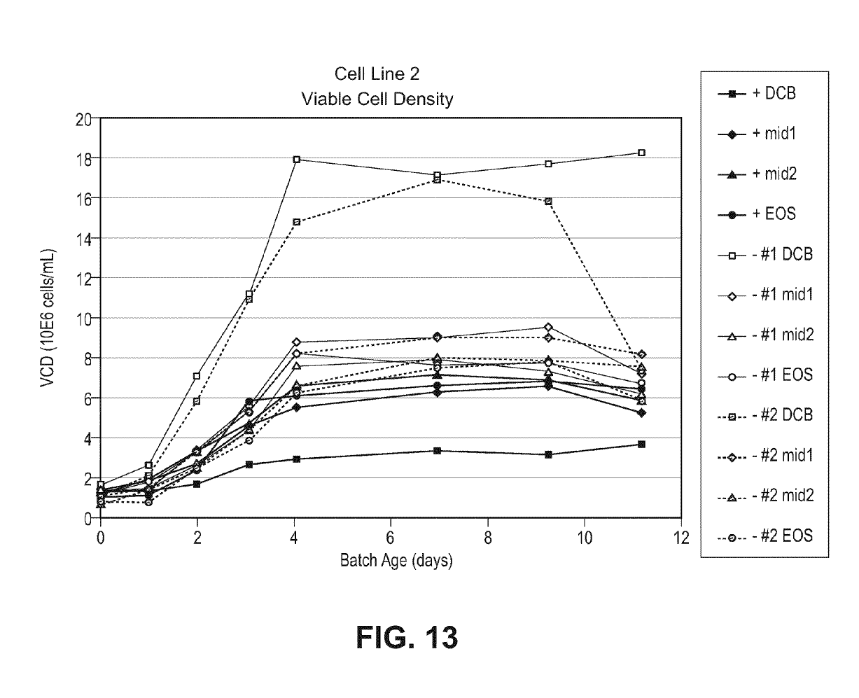

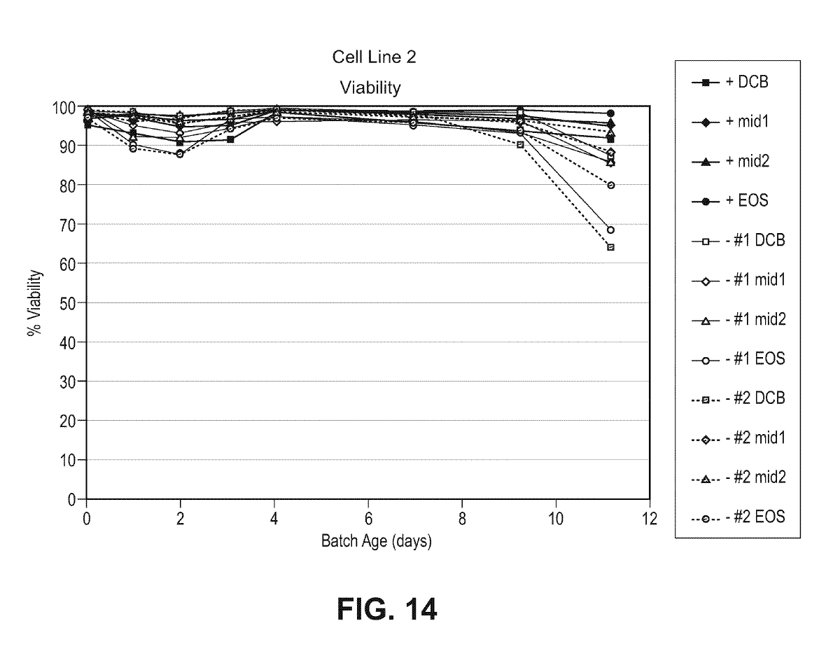

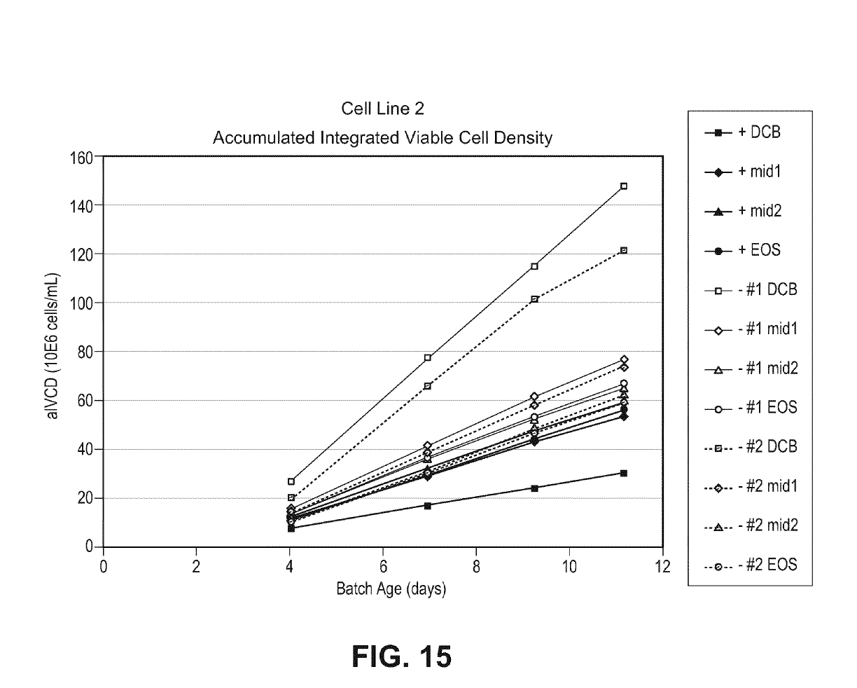

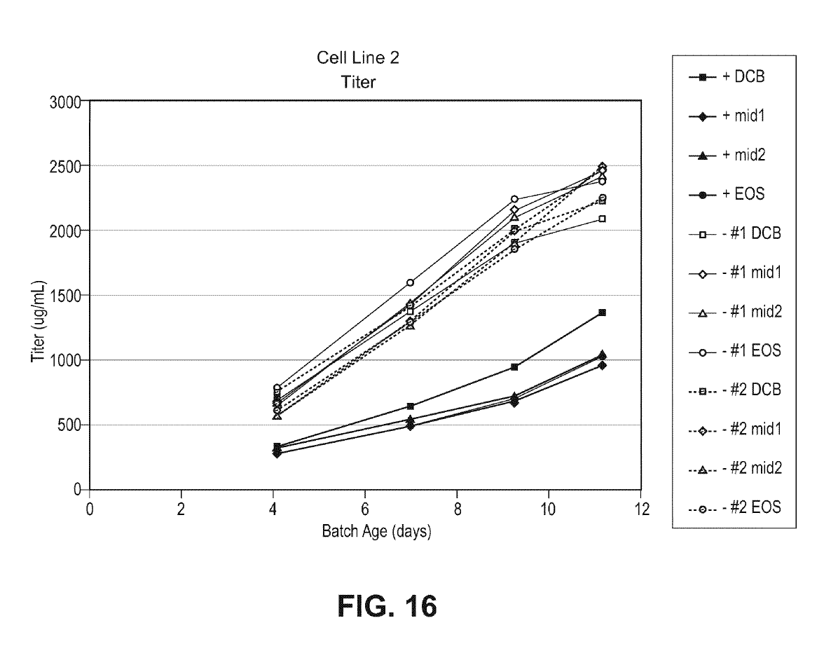

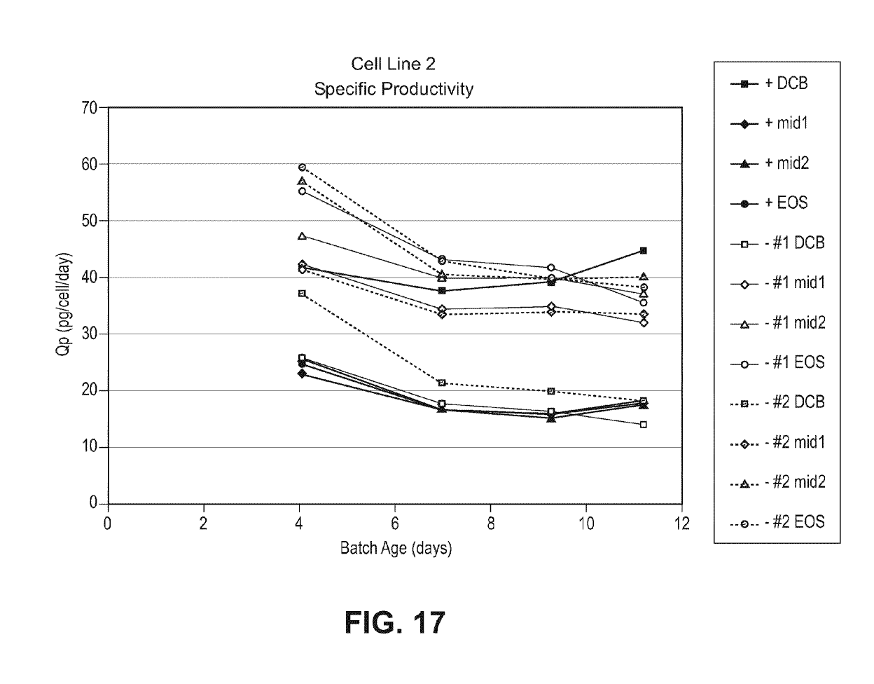

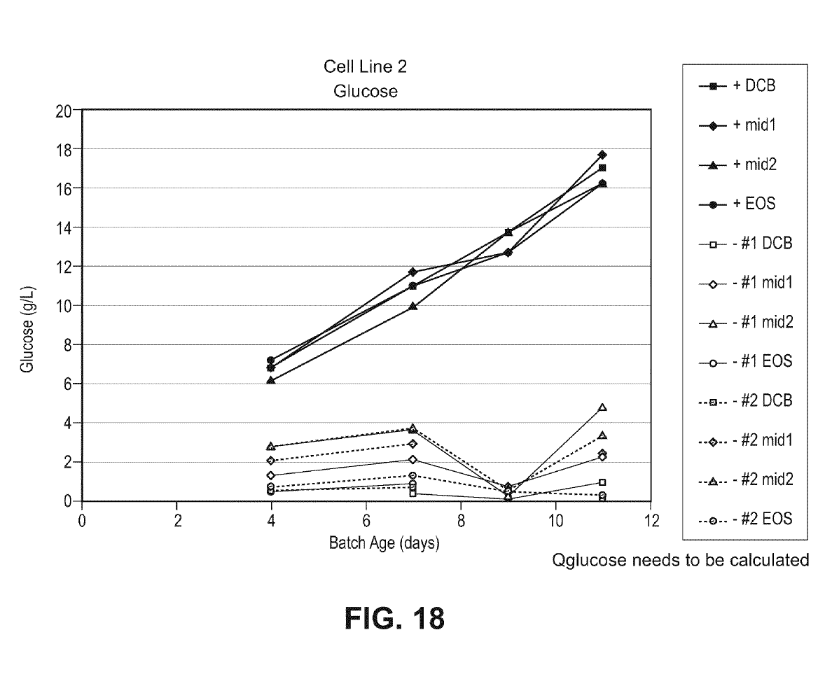

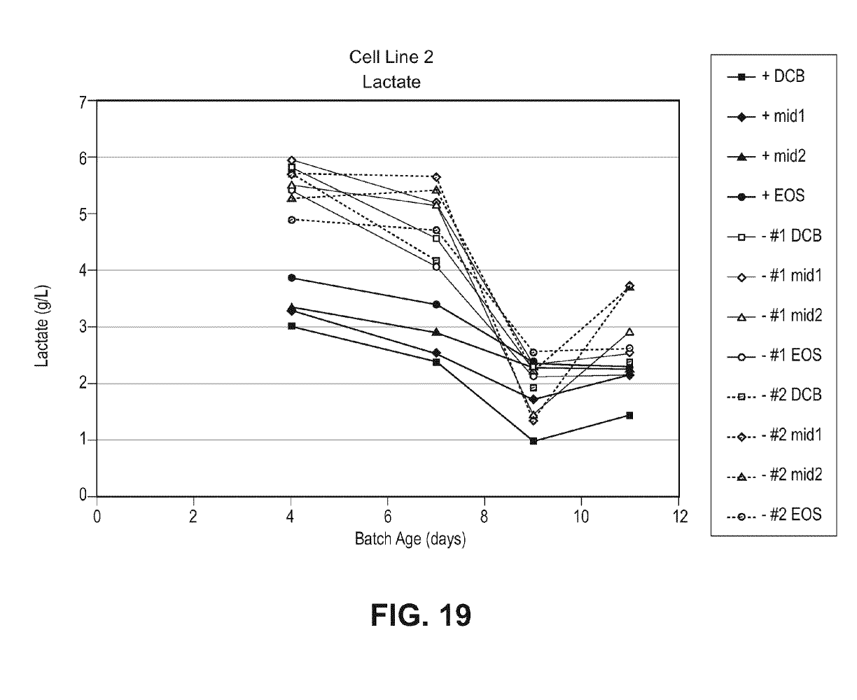

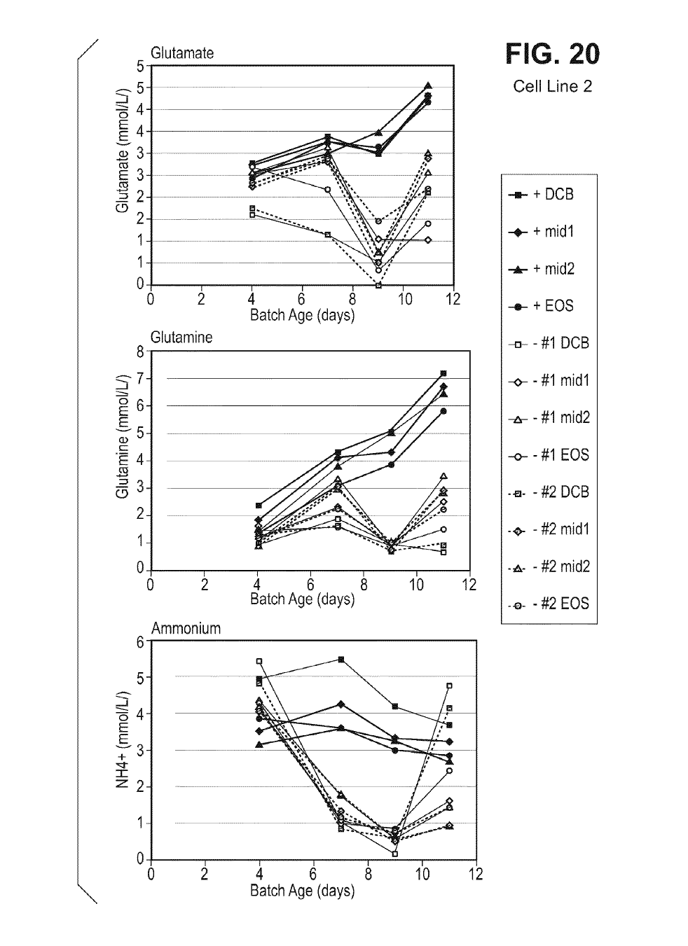

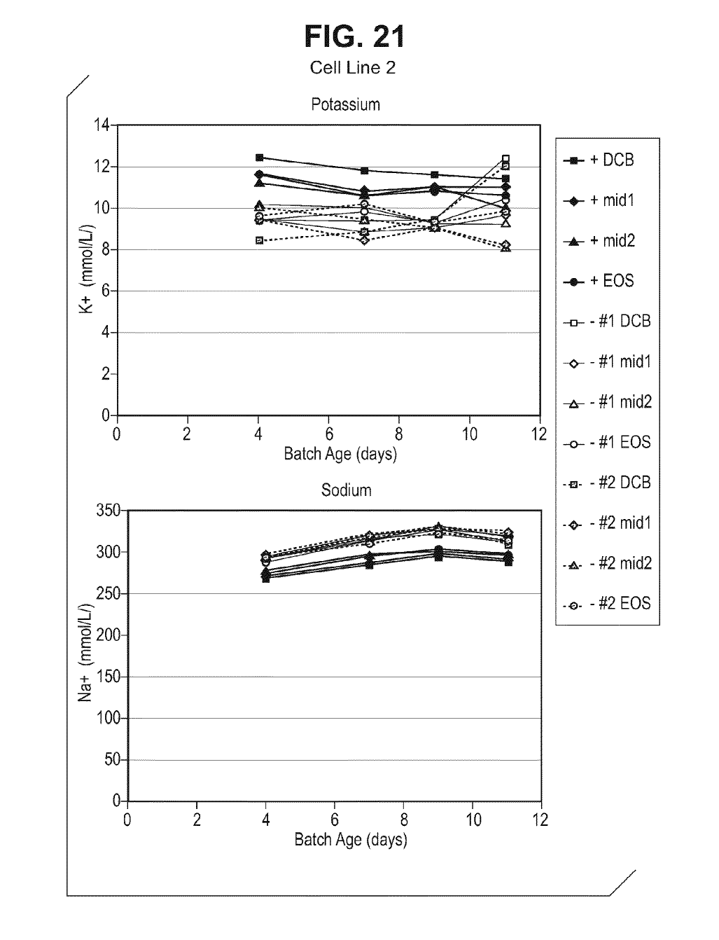

Exemplary results on viable cell density, viability, accumulated integrated viable cell density (aIVCD), specific productivity, titer, nutrient utilization and metabolic waste accumulation are shown in FIGS. 8-21. In this experiment, viable cell density was measured by Guava Cell Counter. aIVCD was measured by determining the average density of viable cells over the course of the culture multiplied by the amount of time the culture has run. Specific productivity was measured by determining the total amount of recombinantly expressed protein produced by the cell culture in a given amount of medium volume. Titer was measured by interferometry using an ForteBio Octet instrument. Nutrient utilization and metabolic waste accumulation levels were measured by detecting concentrations of glucose, lactate, glutamate, glutamine, ammonium, sodium or potassium in cell culture medium.

The results showed that minimal differences were seen in fed batch cultures started from adapted cells taken from DCB to EOS during adaptation process. Importantly, cultures from insulin-free adapted cells produced 2×-3× titer when placed in high cell density fed batch process, as compared to control cultures with cells that were not adapted. Cultures from insulin-free adapted cells also had higher IVCDs (e.g., average increase of 50% over completely insulin-free production and average increase of 30% over existing insulin-dependent platform). Cultures from insulin-free adapted cells also showed differences in nutrient utilization when compared to insulin-dependent cells. For example, cultures of insulin-free adapted cells utilized more glucose.

Experiment 2

This experiment was designed to test re-addition of various concentrations of insulin and LR3 (synthetic IGF-1) in fed batch culture. In this experiment, Medium C was used as base medium and Medium B was used as feed medium. Target seed density was 0.5×10⁶ cells/mL. pH was adjusted post-temperature shift at days 7, 9 or 11. Supplemental feed was added to the cultures on day 4, 7, and day 9.50% glucose was fed to cultures if necessary. Cells were grown in 15 mL cultures. Novo insulin or LR3 (synthetic IGF-1) was supplemented into insulin-free base and/or feed media so that media lots were the same for the experiments. Various concentrations of insulin and LR3 used in the base and/or feed media are summarized in Table 1. This study was designed for concentration range finding exploration and to test the following factors: base insulin, base LR3 growth factor, feed insulin, feed LR3 growth factor. Condition #1, which was a completely insulin-free culture, was used as baseline control. Condition #2 was an insulin-dependent platform control. 50% glucose was fed at 5 g/L to cells cultured under condition #4 on day 11. Cells used in this experiment express a monoclonal antibody.

TABLE 1
Exemplary insulin/LR3 study design

| | | | Base | | Feed |
|---|---|---|---|---|---|
| | | | LR3 | | LR3 |
| | | Base | Growth | Feed | Growth |
| | | Insulin | Factor | Insulin | Factor |
| Adaptation | | Concentration | Conc. | Conc. | Conc. |
| (±Insulin) | Condition | (mg/L) | (ug/L) | (mg/L) | (ug/L) |
| − | 1 | 0 | 0 | 0 | 0 |
| + | 2 | 10 | 0 | 165 | 0 |
| − | 3 | 0 | 0 | 0.165 | 0 |
| − | 4 | 0 | 0 | 165 | 1.67 |
| − | 5 | 0 | 5 | 0 | 0.167 |
| − | 6 | 0 | 50 | 0.165 | 1.67 |
| − | 7 | 0 | 50 | 16.5 | 0.167 |
| − | 8 | 1 | 0 | 0 | 1.67 |
| − | 9 | 1 | 0 | 16.5 | 0 |
| − | 10 | 1 | 5 | 0.165 | 0 |
| − | 11 | 1 | 5 | 1.65 | 0.167 |
| − | 12 | 1 | 50 | 165 | 0.167 |
| − | 13 | 10 | 0 | 0.165 | 0.167 |
| − | 14 | 10 | 5 | 16.5 | 1.67 |
| − | 15 | 10 | 5 | 165 | 0 |
| − | 16 | 10 | 50 | 0 | 0 |
| − | 17 | 10 | 50 | 1.65 | 0.167 |

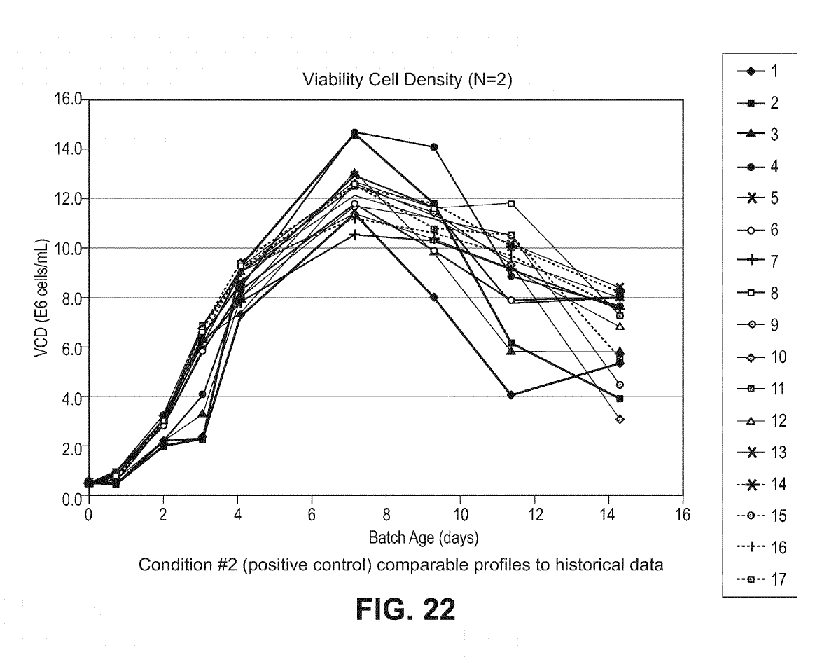

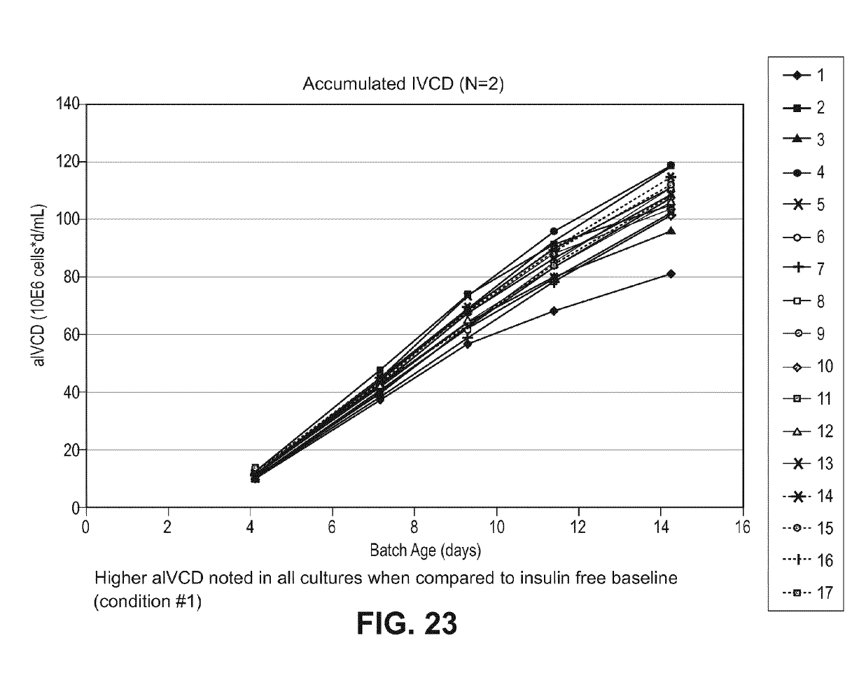

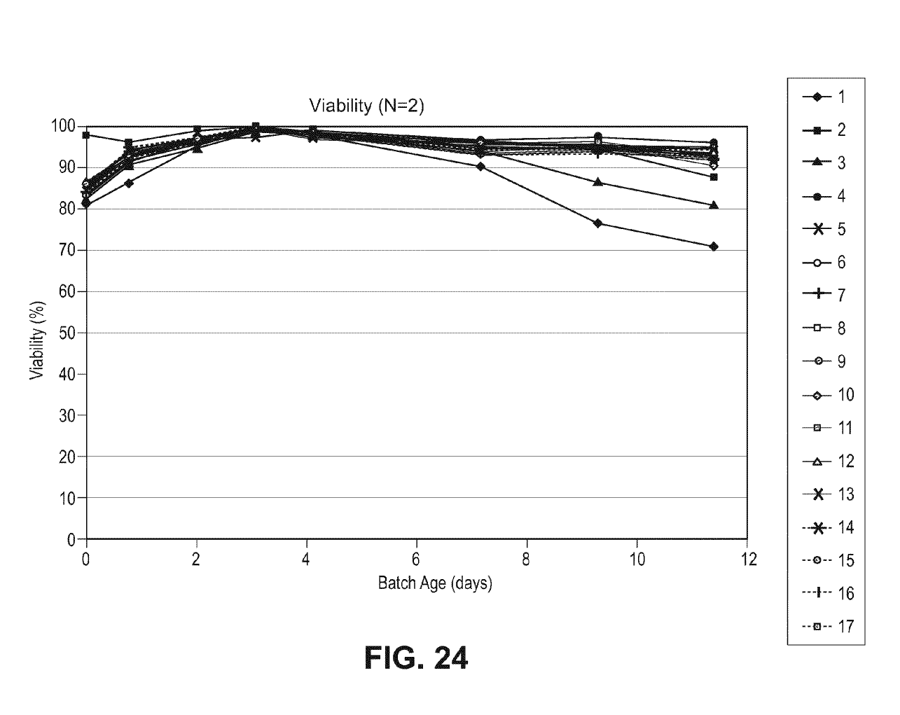

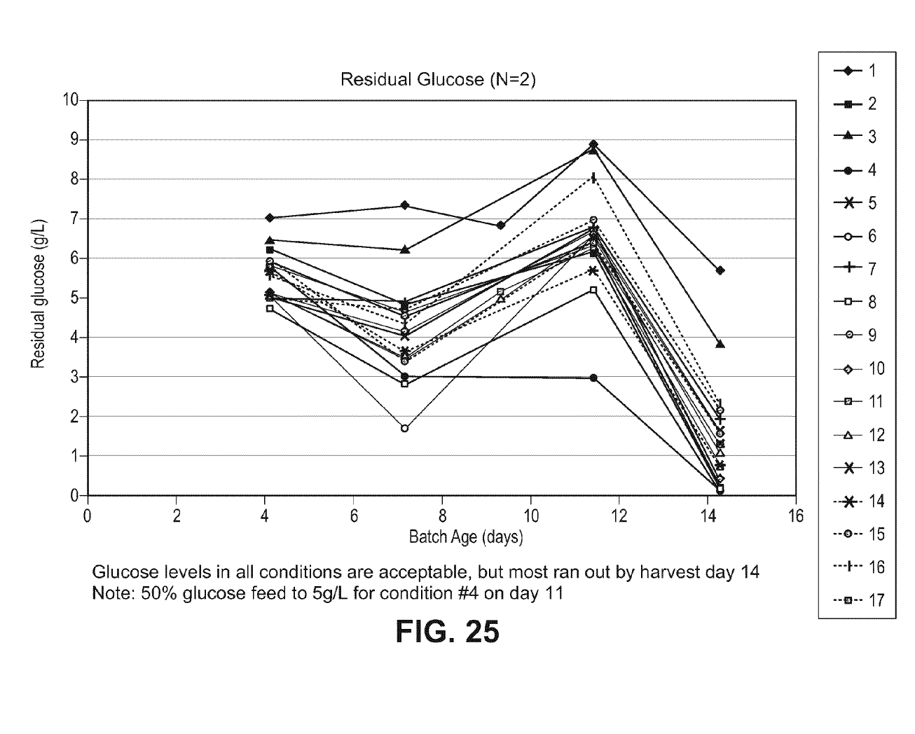

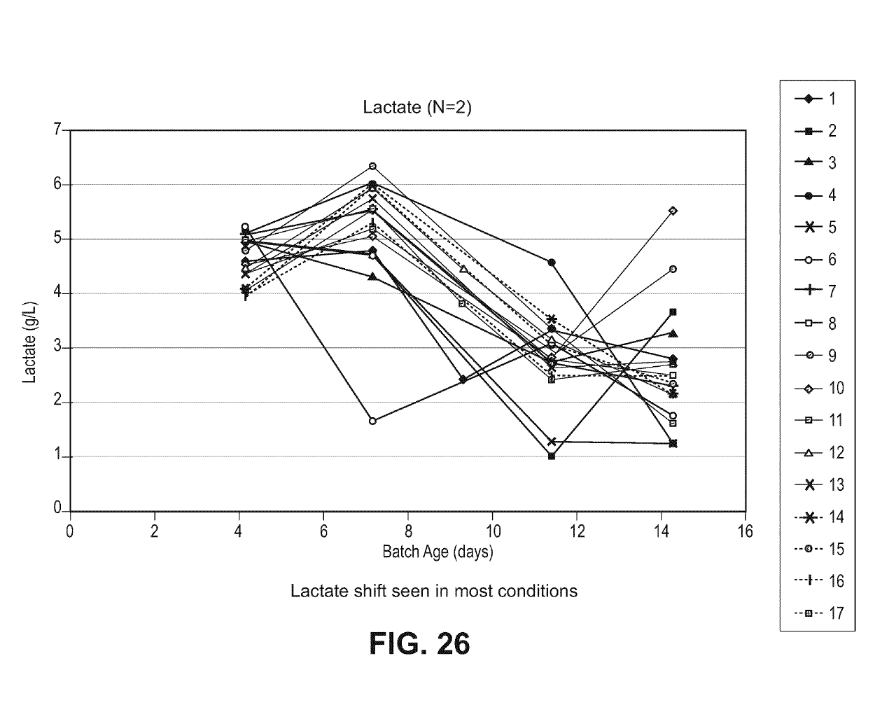

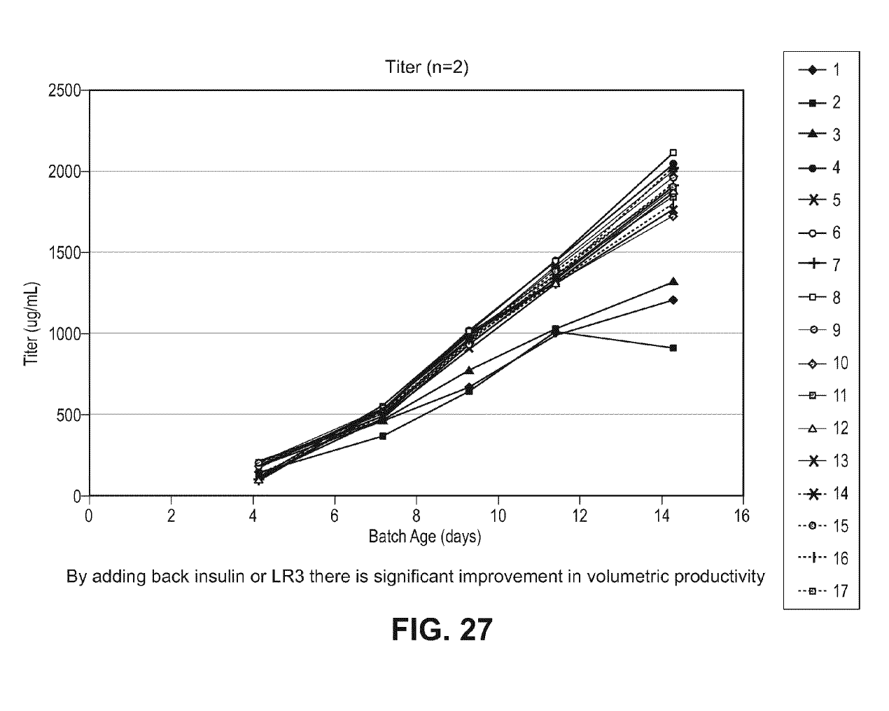

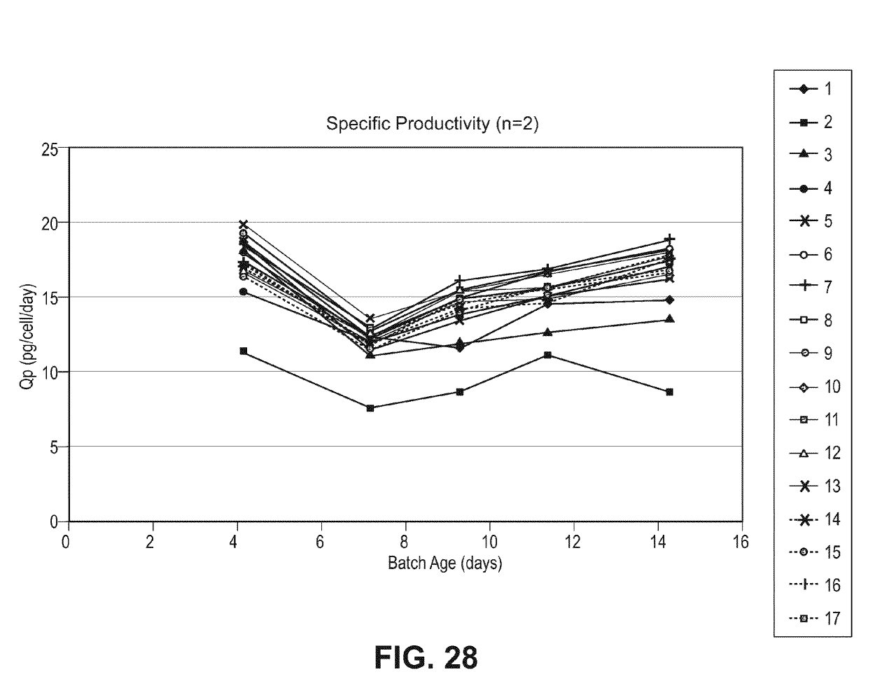

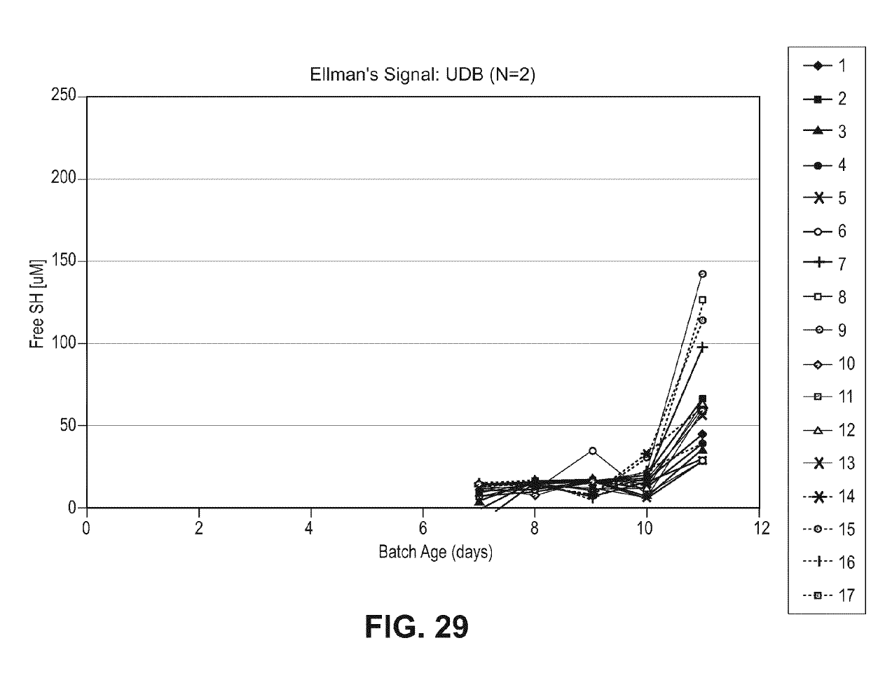

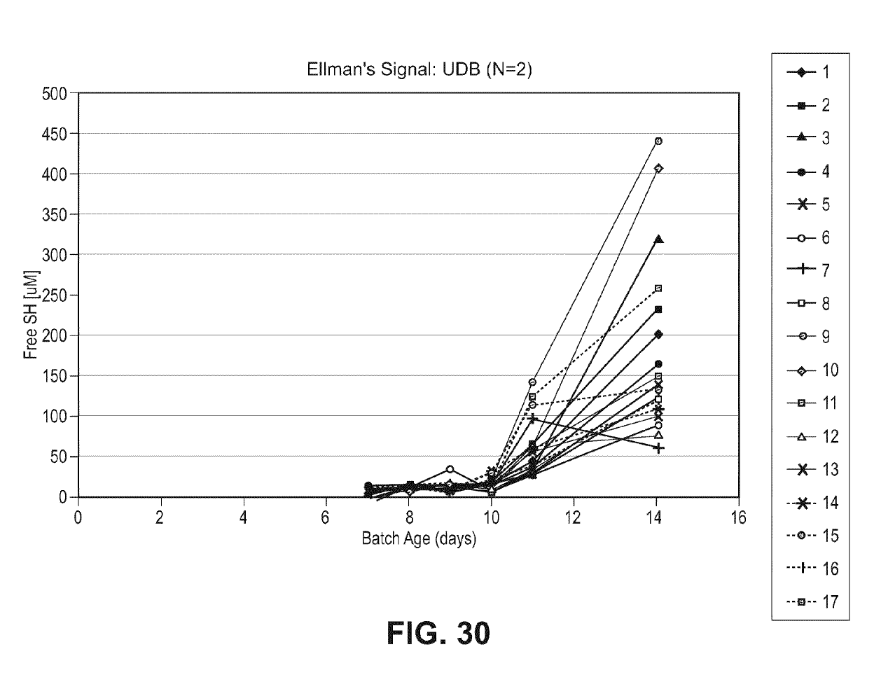

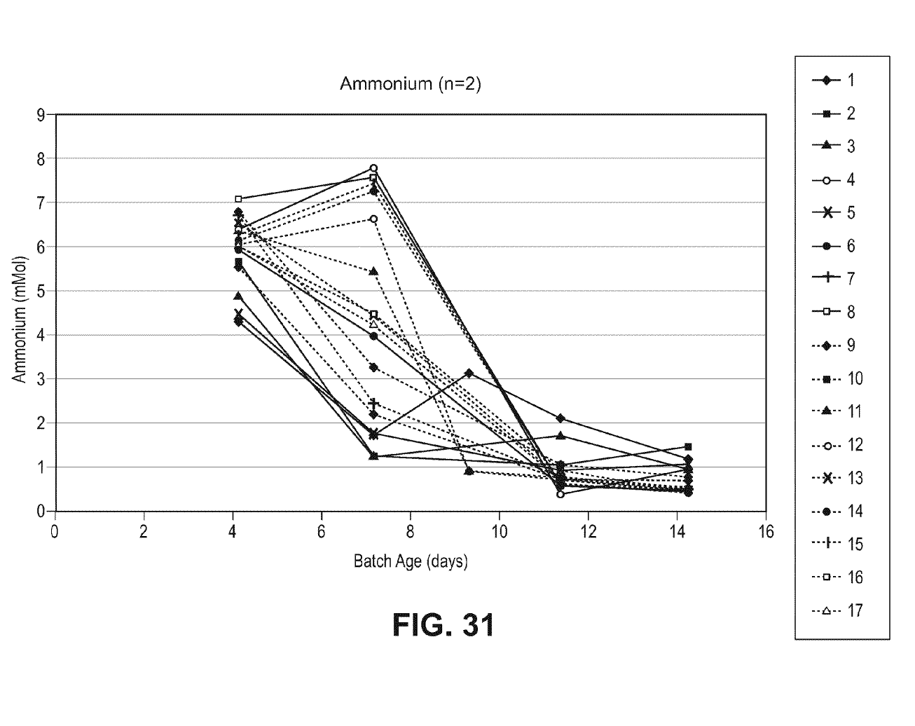

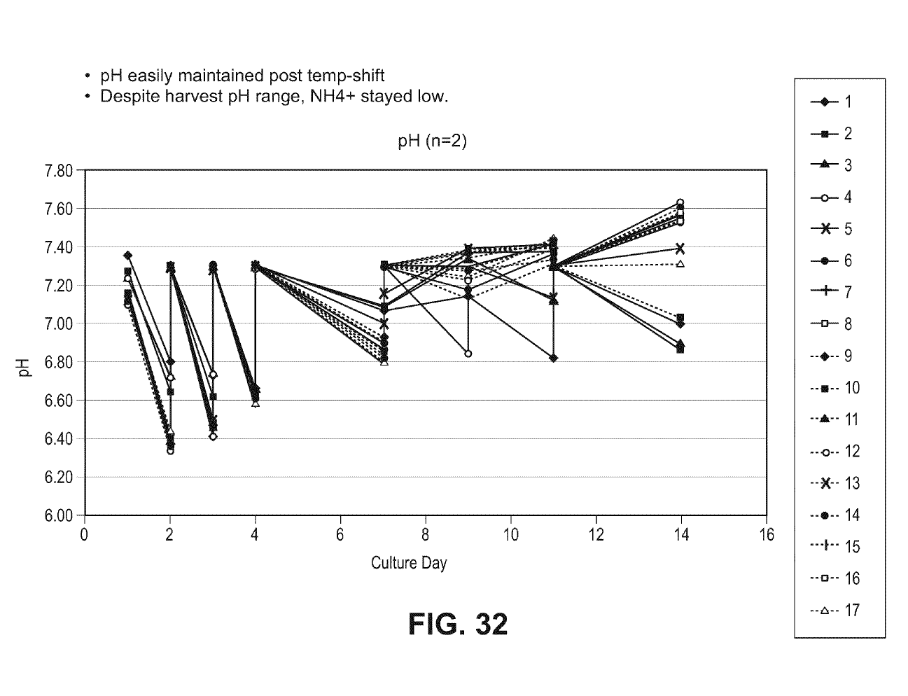

Endpoints collected included GUAVA, pH, metabolic analysis (NOVA), Osmo, Titer (Octet), insulin, Ellman's, product quality (e.g., SEC, N-glycans). Exemplary results are shown in FIGS. 22-32. The results showed that positive control (condition #2) demonstrated comparable profiles to historical data (FIG. 22). Higher aIVCD was observed in all cultures when compared to insulin free baseline (condition #1) (FIG. 23). Completely insulin-free condition has lowest harvest viability and density (condition #1) (FIG. 24). Viability may be negatively affected with very low insulin concentrations (e.g., condition #3=0, 0, 0.165, 0). Glucose levels in all conditions were acceptable, but generally ran out by harvest day 14 (FIG. 25). Lactate shift seen in most conditions (FIG. 26). By adding back insulin or LR3, there is significant improvement in volumetric productivity (FIG. 27). The specific cellular productivity of insulin-free adapted cells is increased compared to that of the insulin-dependent control cells (FIG. 28).

Experiment 3

In this experiment, adapted cells expressing a monoclonal antibody were cultivated in fed batch with re-addition of insulin or LR3. In this experiment, adaptation medium contained 10 mg/L insulin, base medium contained 10 mg/L insulin, and feed medium contained 165 mg/L insulin. LR3 (synthetic IGF-1) was supplemented into feed media so that media lots at a concentration of 50 ng/mL in culture per feed.

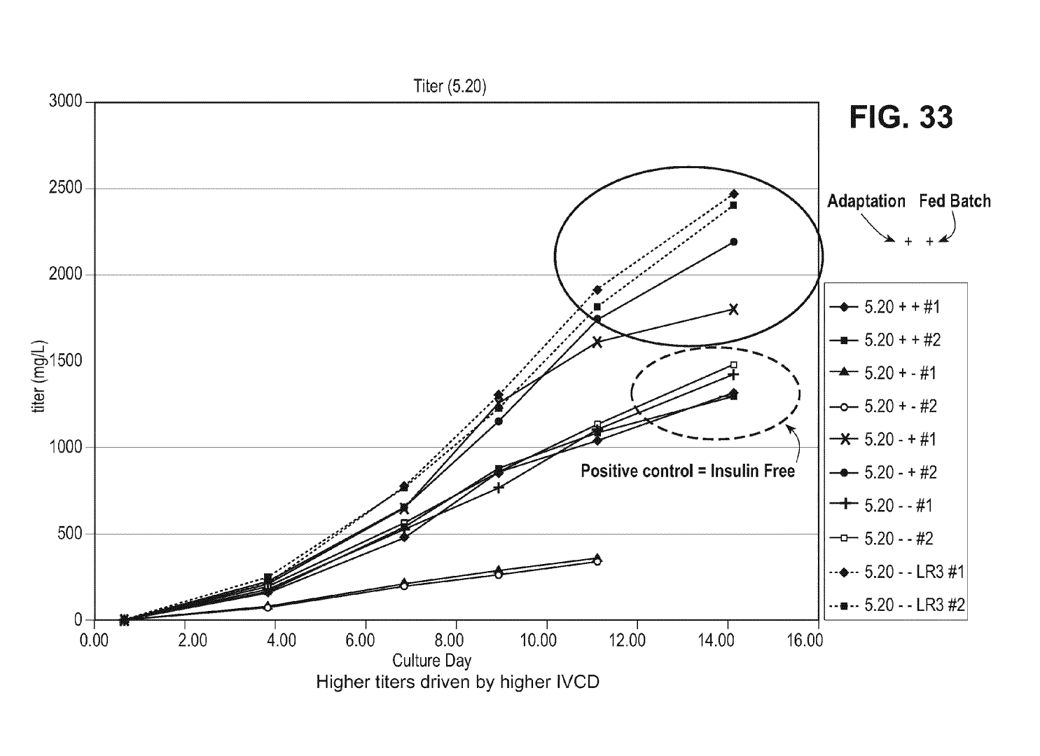

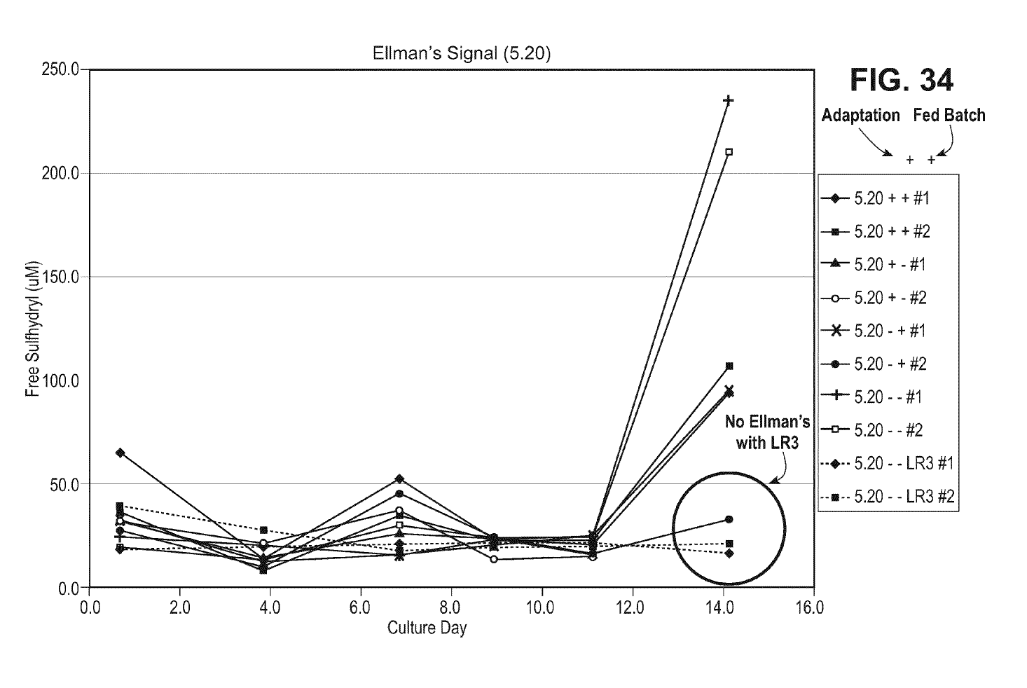

Exemplary results on titer and Ellman's Signal are shown in FIGS. 33 and 34. Highest titers were seen in insulin-free adapted cells plus insulin or LR3 in production (FIG. 33). No Ellman's signals in production cultures using insulin-free adapted cells with LR3 re-addition (FIG. 34). Ellman's assays measure free sulfhydryl groups in cell culture medium. Reduced Ellman's signal indicates that reduced amount of free sulfhydryl group.

Thus, insulin-free adapted cell cultures demonstrated delayed increase in Ellman's measurements of the cell culture medium compared to control cells, indicating lower levels of free sulfhydryl groups in the medium.

In general, ideal performance were seen in insulin-free adapted culture plus LR3. It has highest day 14 titer, cellular productivity (Qp) and integrated viable cell density (IVCD). Viability was maintained above 85% through day 14. Delayed Ellman's signal rise and no late stage lactate production were observed. Insulin free adapted cultures plus insulin have comparable titer, Qp and IVCD to insulin-free cultures with LR3. Ellman's rise was delayed at platform insulin concentrations. Best growth at 37° C. Insulin-free adapted cultures without insulin showed slowest growth at 37° C. with low IVCD. It also has early Ellman's signal rise.

Experiment 4

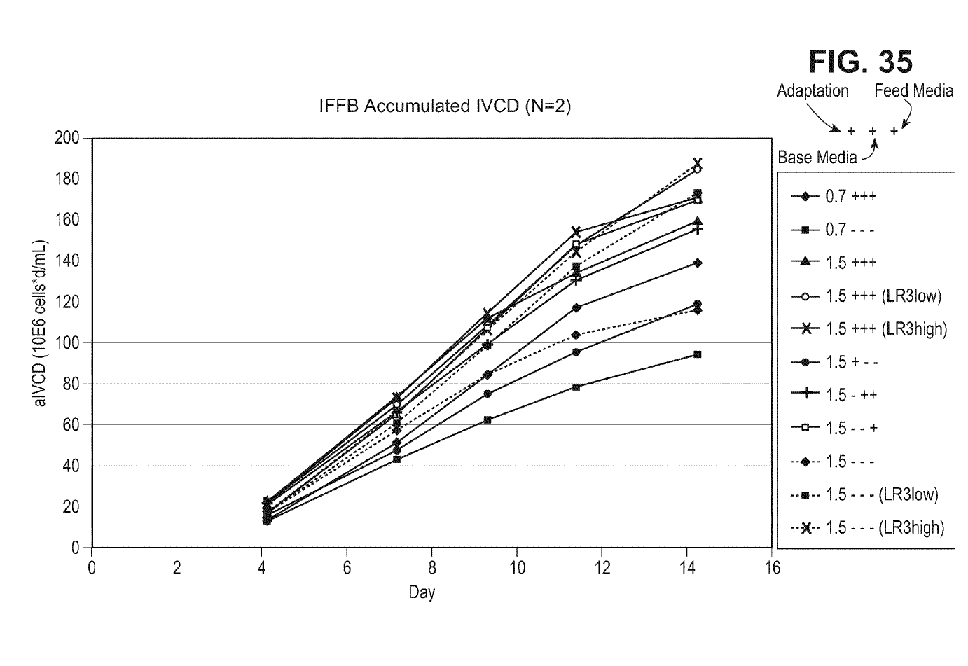

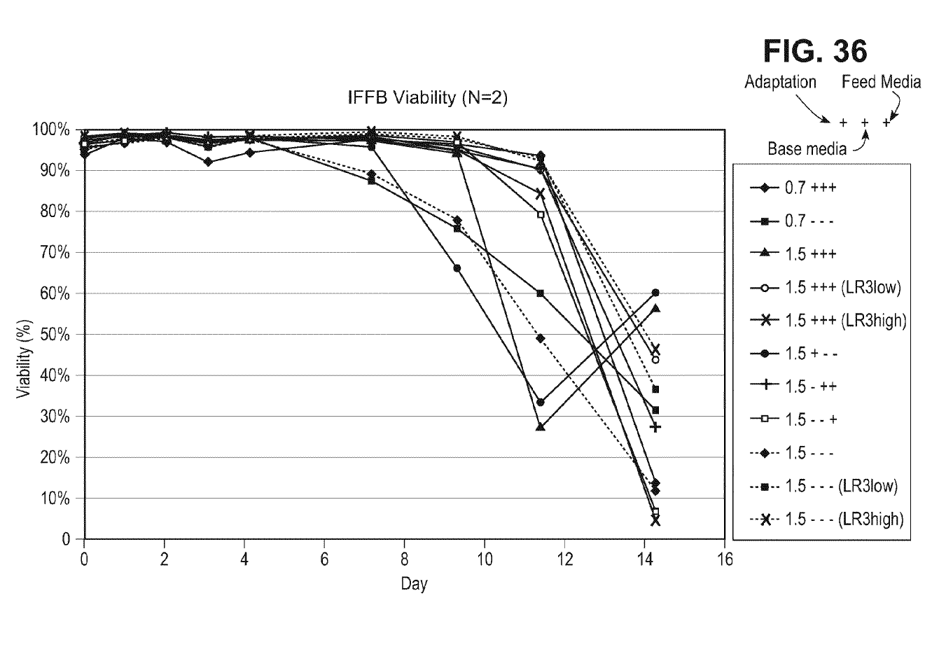

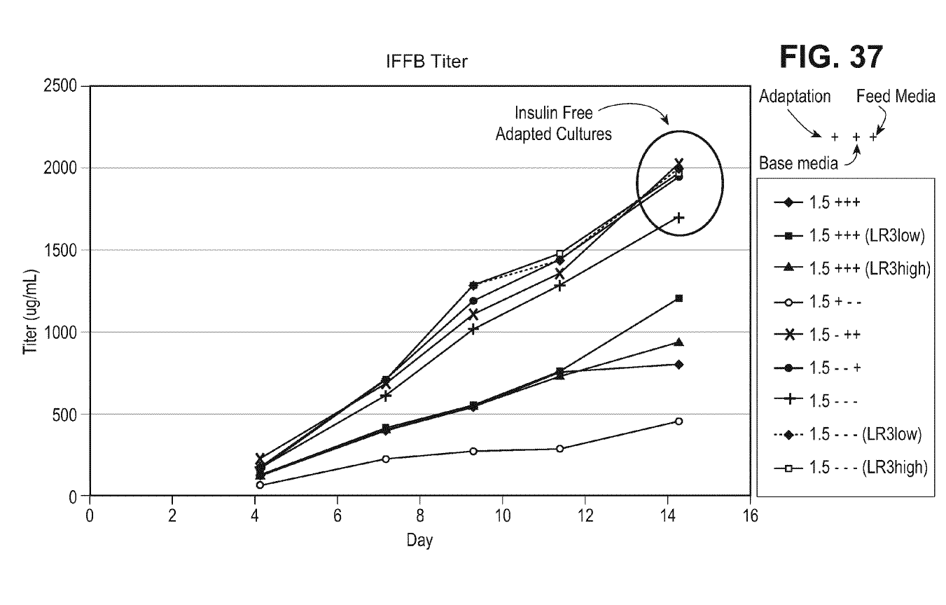

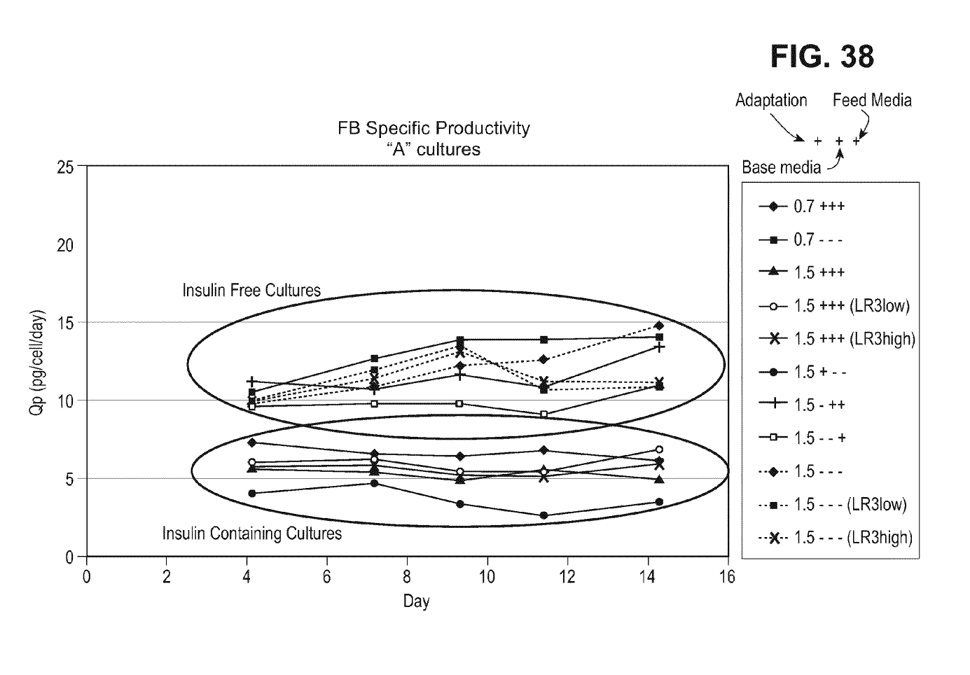

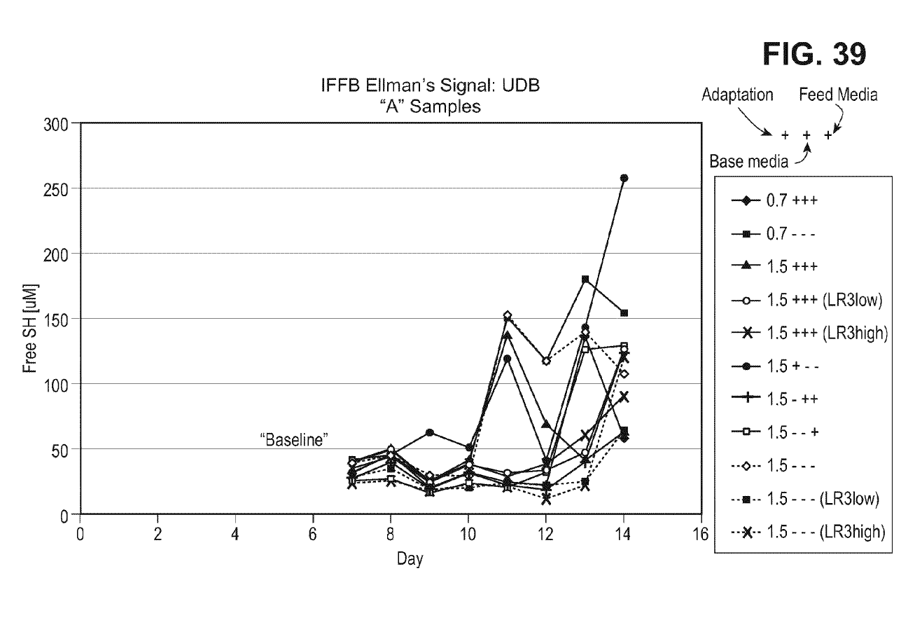

This experiment was designed to further test re-addition of LR3 with supplemental feeds. Standard pH-adjusted fed batch was used. Medium C (+/−insulin) was used as base medium. Medium B (+/−insulin) was used as feed medium. LongR3 was added with supplemental feeds at a low level of 50 ng/mL or a high level of 150 ng/mL. Two seed densities were used: 0.7e6 cells/mL and 1.5e6 cells/mL. pH adjusted post temperature shift on sample days 7, 9 and 11. Cells used in this experiment express a monoclonal antibody. Exemplary results illustrating integrated viable cell density, viability, titer, specific productivity, Ellman's signals, are shown in FIGS. 35-39.

In summary, control cultures routinely passaged with insulin and then put in a fed batch performed as expected. Cultures not adapted to insulin-free media performed poorly when placed into a fed batch without insulin. Adding insulin back shows improvement to production. LongR3 addition aided in culture growth.

Experiment 5

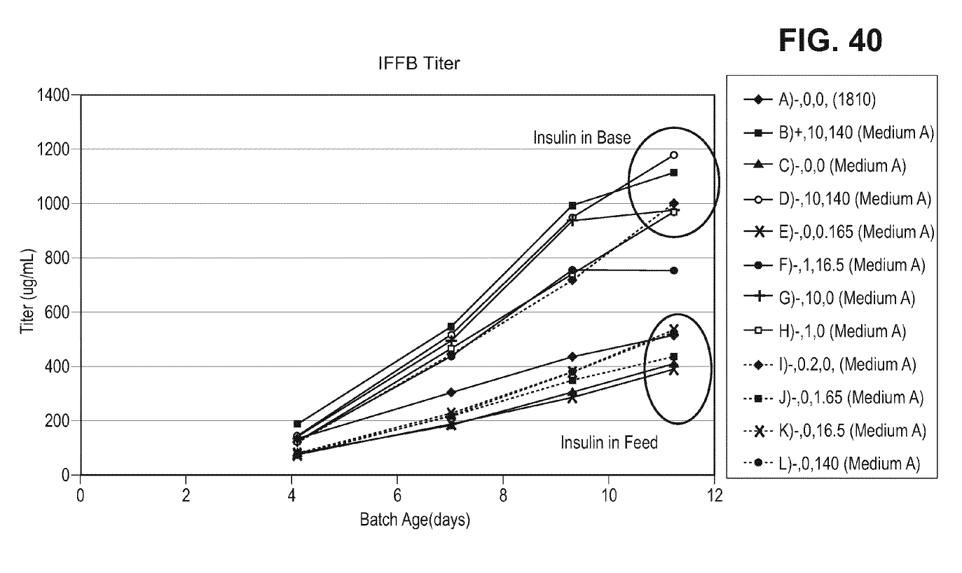

This experiment was designed to test if insulin is more effective in the base or feed medium and to ask how low we can go with insulin. Base medium (+/−insulin) contained MEDIUM A basal medium supplemented with amino acids. Medium B was used as feed medium (+/−insulin). We tested 4 base insulin levels (0, 0.2, 1, 10 mg/L) and 5 feed insulin levels (0, 0.165, 1.65, 16.5, 140 mg/L). Cells cultured in this experiment express a monoclonal antibody. Exemplary results on titer are shown in FIG. 40. As can be seen, insulin added back in the base was more effective in this experiment. The cells also showed some dose-response to insulin levels.

Experiment 6

This experiment was designed to test additional insulin concentration levels in base and/or feed media. Base medium (+/−insulin) contained Medium A basal medium supplemented with amino acids. Medium B was used as feed medium (+/−insulin). Cells were grown in 15 mL culture volumes. Target seed density was 1.5e6 cells/mL. pH adjusted post-temperature shift at day 7, 9 and 10. Supplemental Feed at day 4 (5%), day 7 (4%), and day 9 (3%). Four different cell lines were cultured in this experiment, including Cell Line 1 expressing an Fc-fusion protein, Cell Line 2 expressing a nanobody, Cell Line 3 and 4 expressing a monoclonal antibody. The following conditions were tested.

Adaptation, Base, Feed
(mg/L insulin)

+, 10, 140
−, 10, 140
−, 0, 0
−, 0.2, 0
−, 1, 0
−, 2, 0

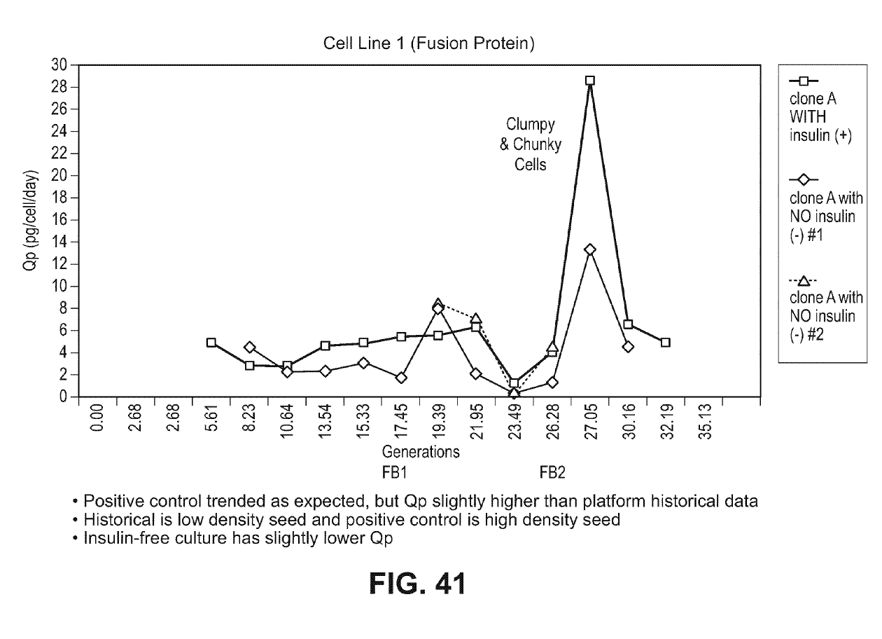

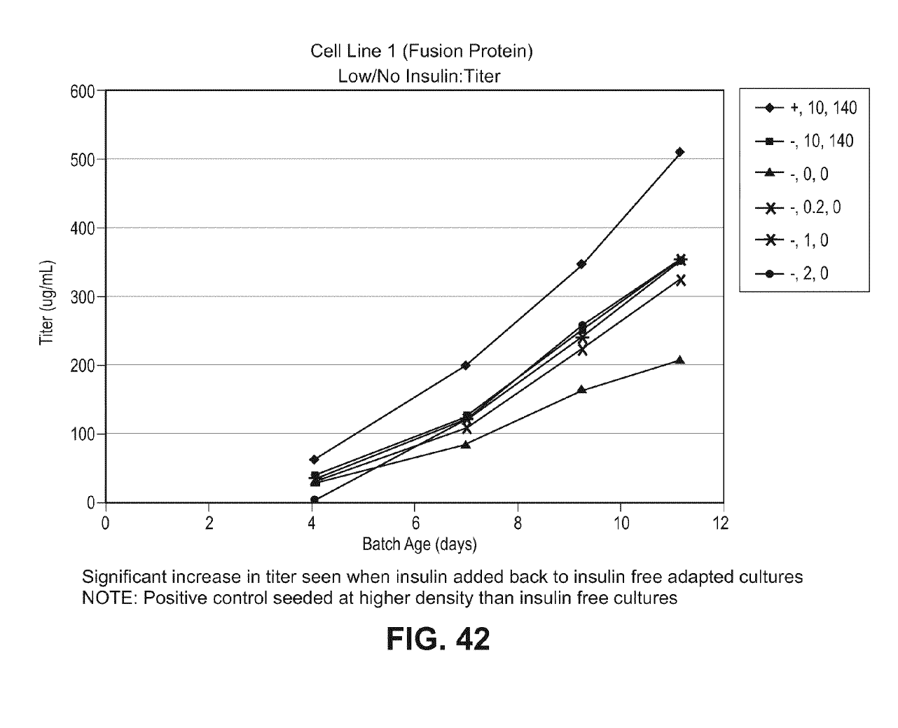

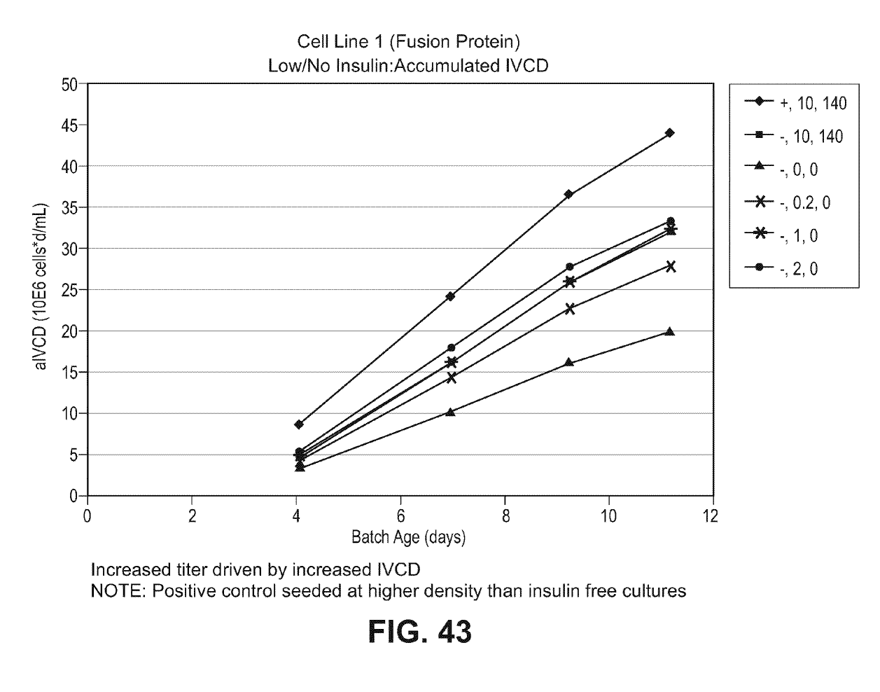

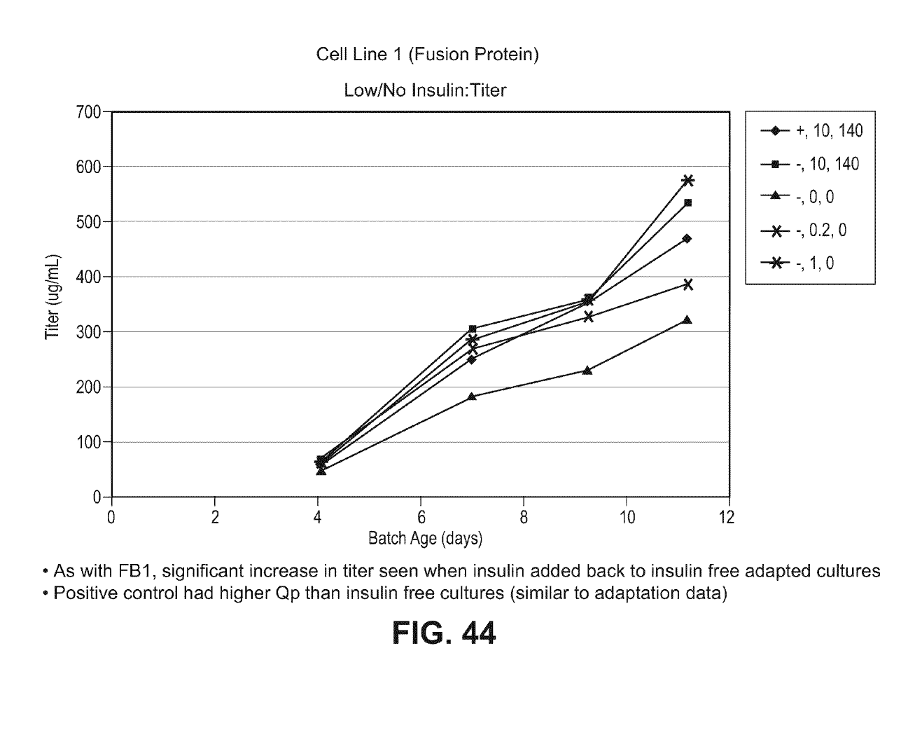

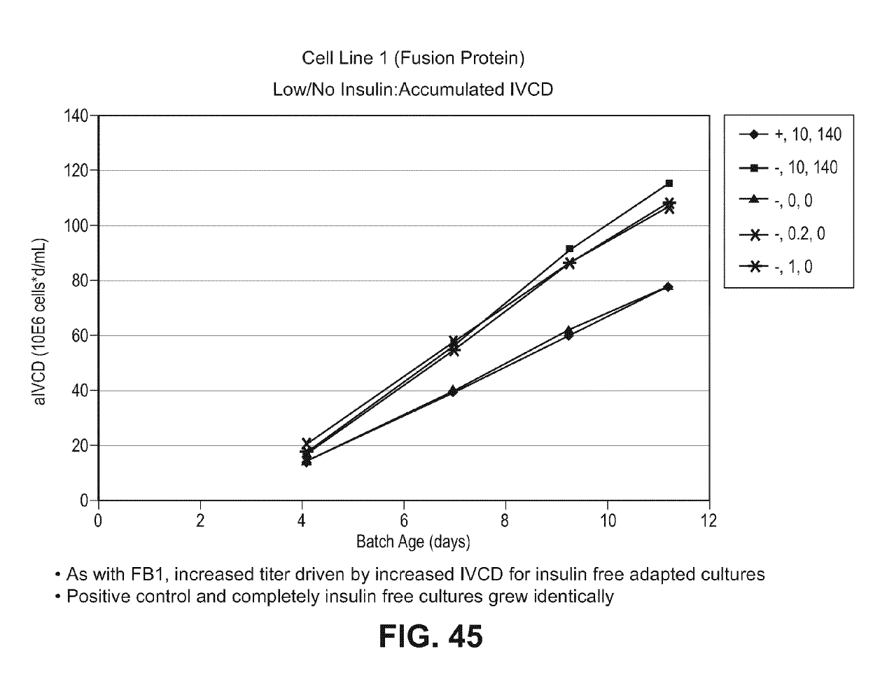

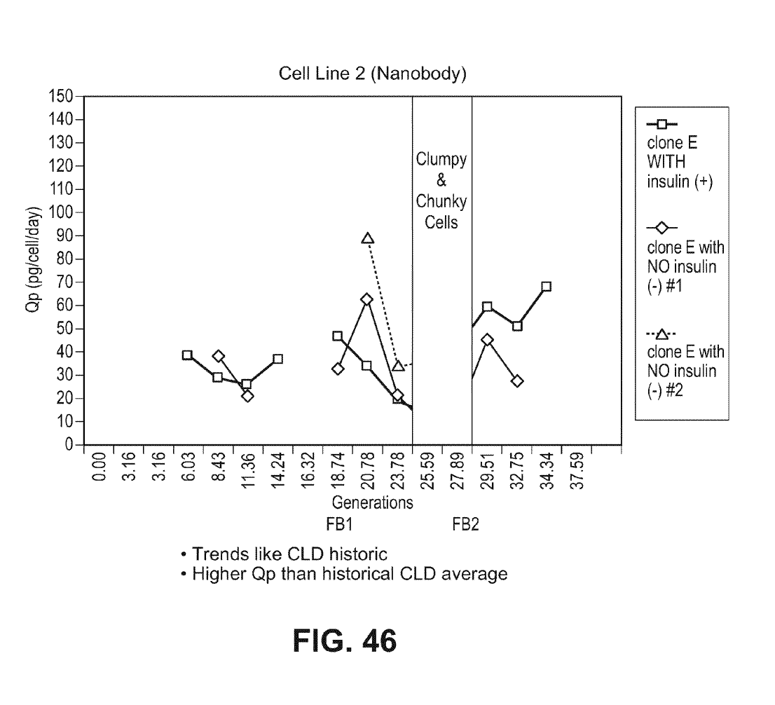

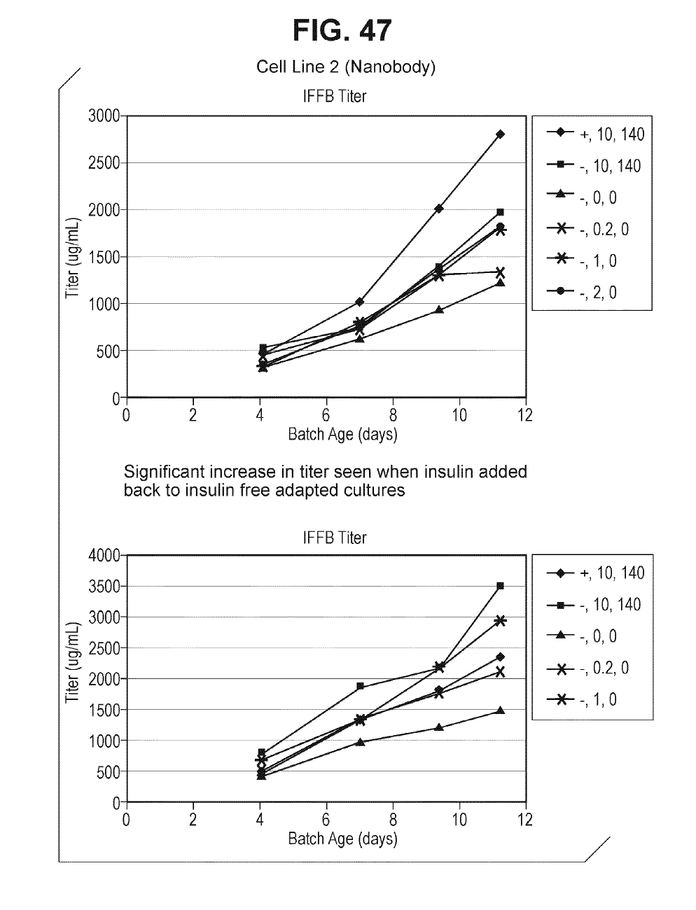

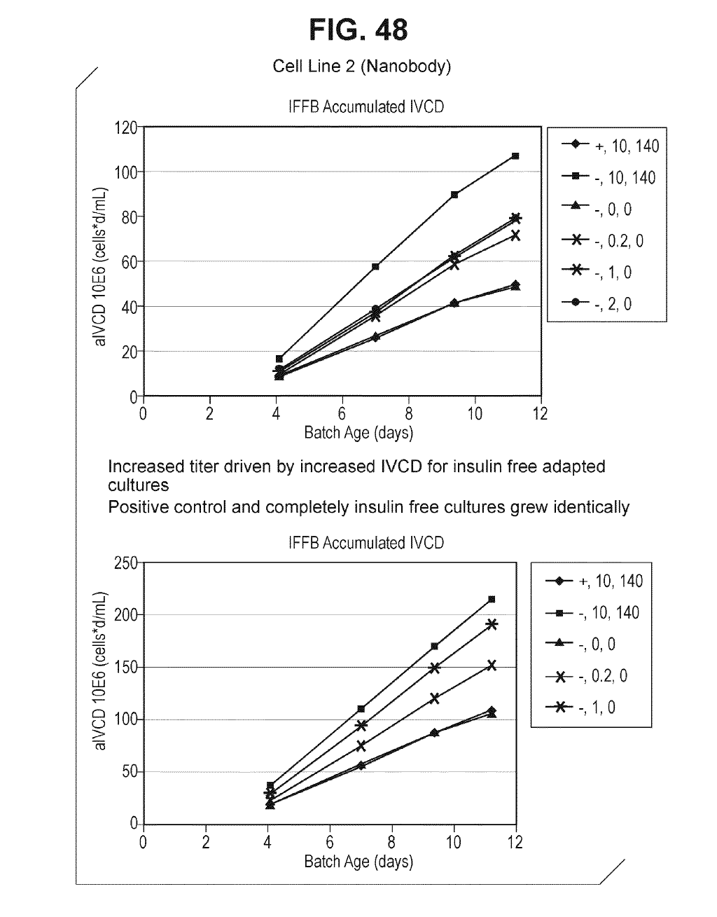

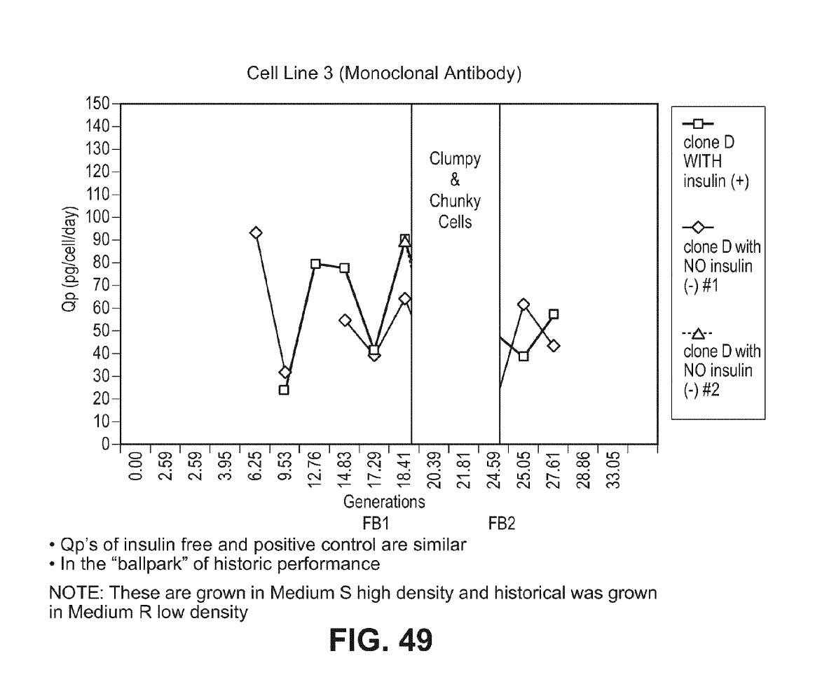

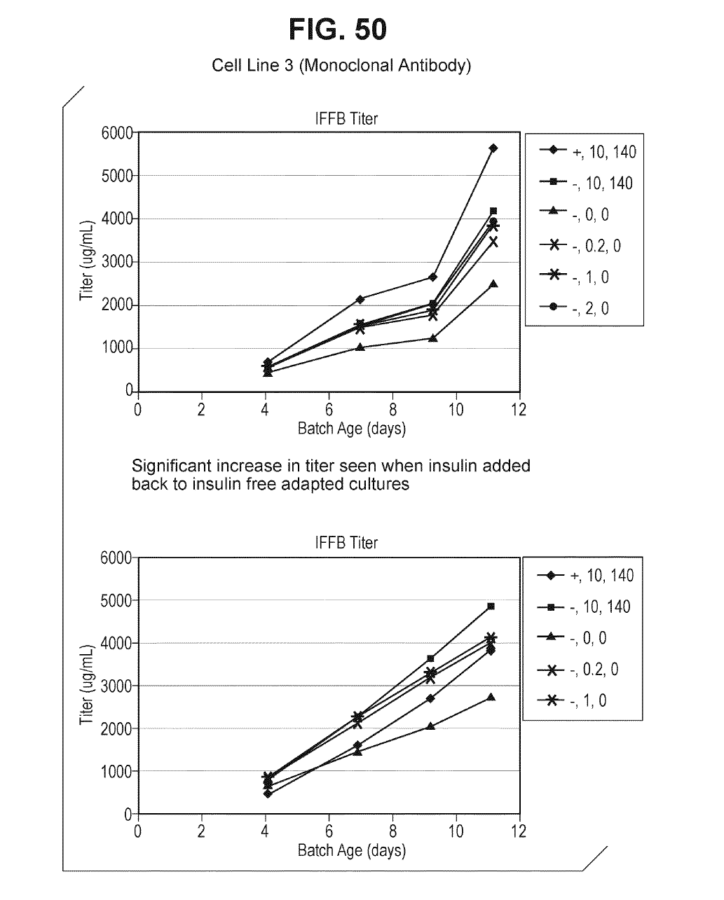

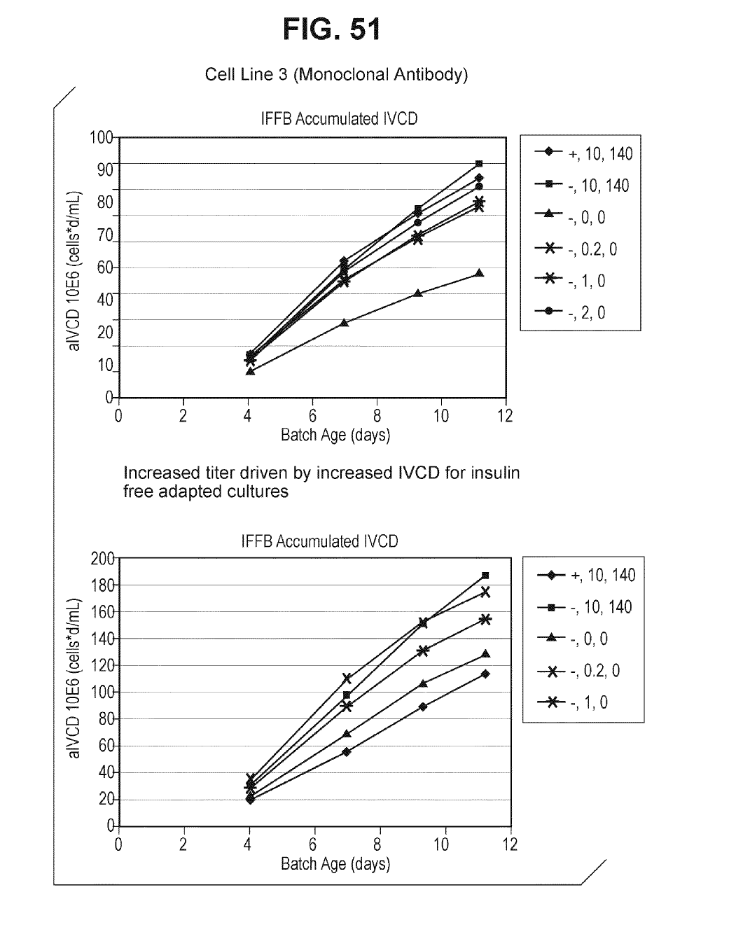

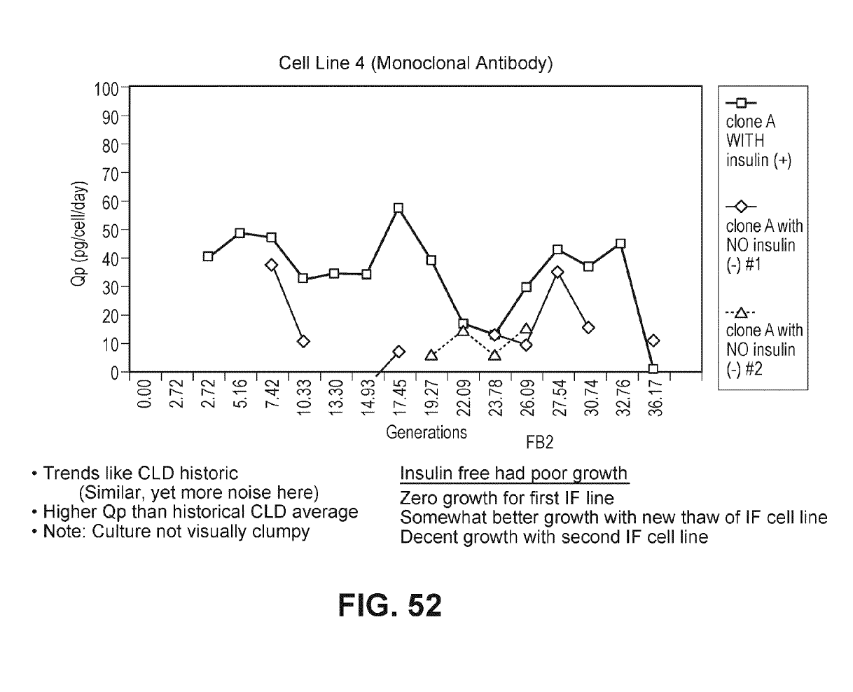

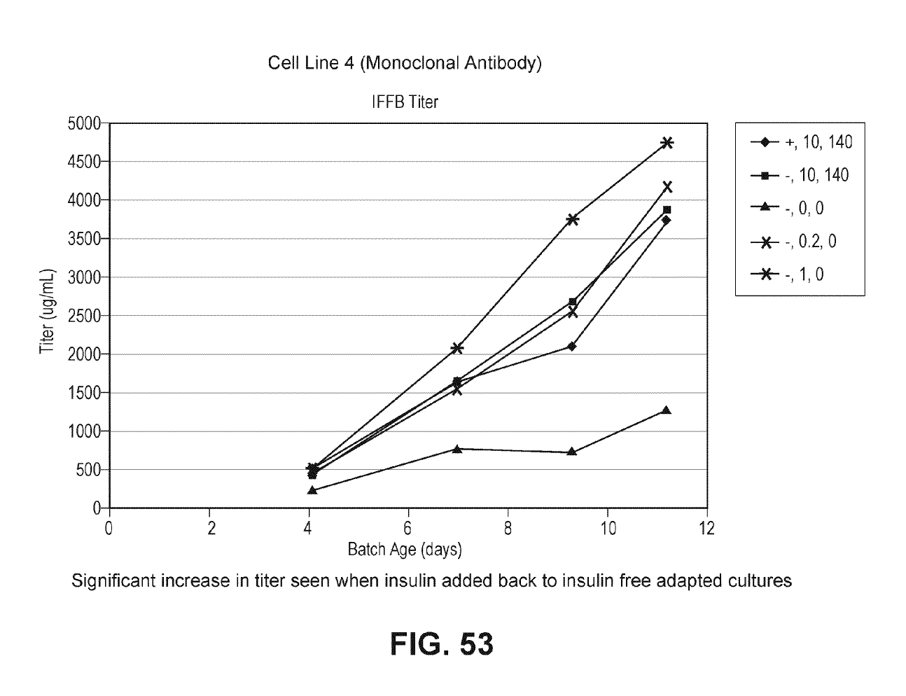

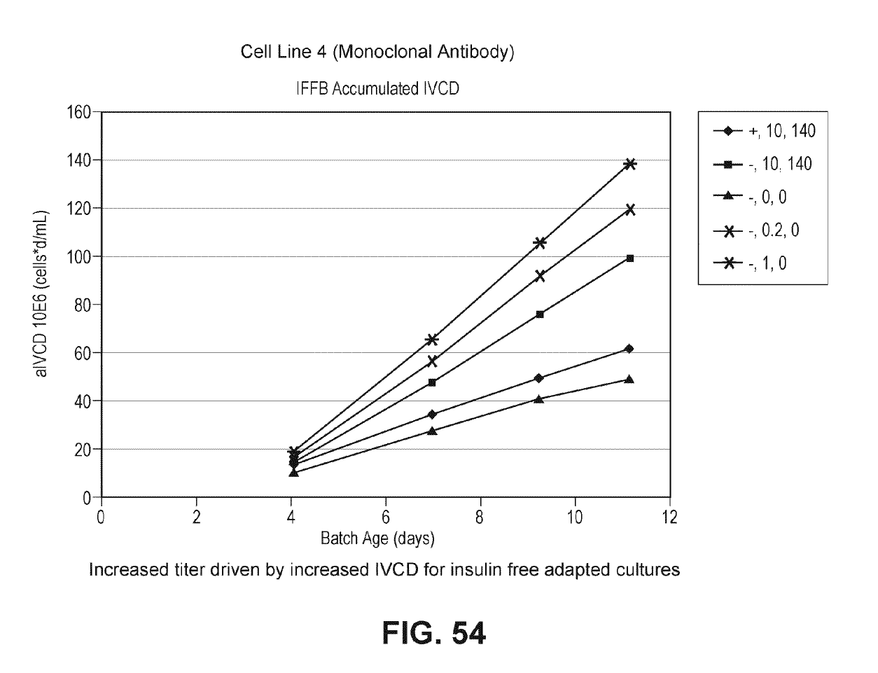

Exemplary results are shown in FIGS. 41-54. The results showed that significant increase in titer was seen when insulin added back to insulin-free adapted cultures in different cell lines. Insulin-free adapted cultures with insulin added-back also displayed increased IVCD. Average increase of growth and titer is about 50% compared to completely insulin-free cultures. Insulin level in the base medium as low as 0.2 mg/L enhanced cell growth and productivity.

### Example 3

### Optimization Using Design-Expert

To further optimize the fed batch culture conditions, Design-Expert® 7.0.1 Software (Stat-Ease, Inc.) was used. Design-Expert® is a software package which uses historical data from a variety of characterization steps to design optimal ranges for cell culture parameters. Typically, such study type is known as response surface historical data. Design model is known as reduced quadratic. Design-Expert® were used for range finding exploration and test factors such as base insulin, base growth factor, feed insulin and feed growth factor.

Typically, the following desirable criteria are used:
Base insulin=minimize
Base growth factor=equal to zero
Feed insulin=minimize
Feed growth factor=equal to zero
Titre=maximize
Qp=maximize
Ellman's=maximize (culture day on which Ellman's rises above baseline)

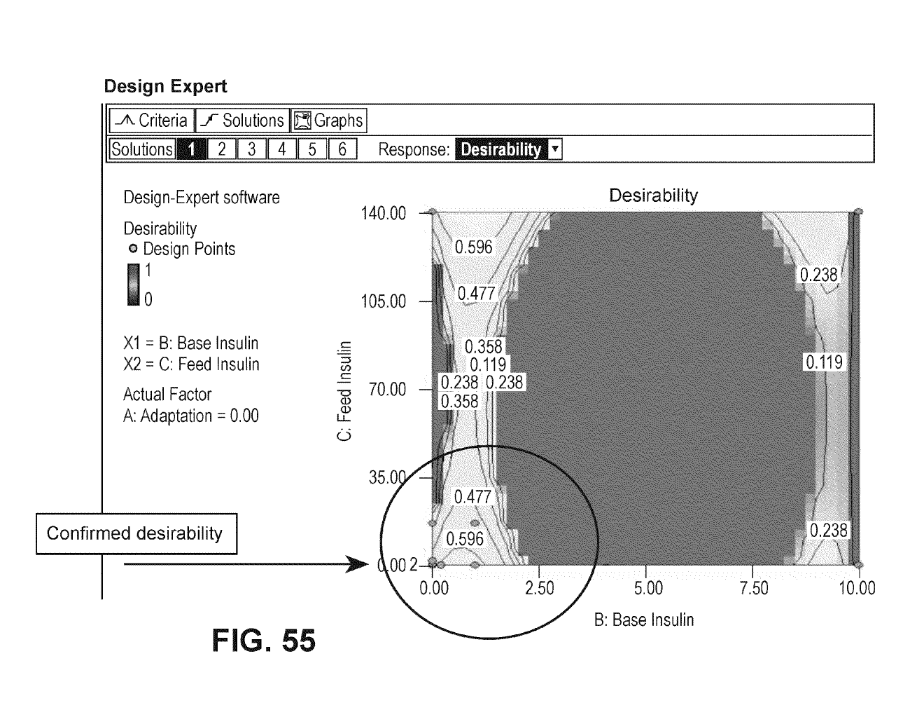

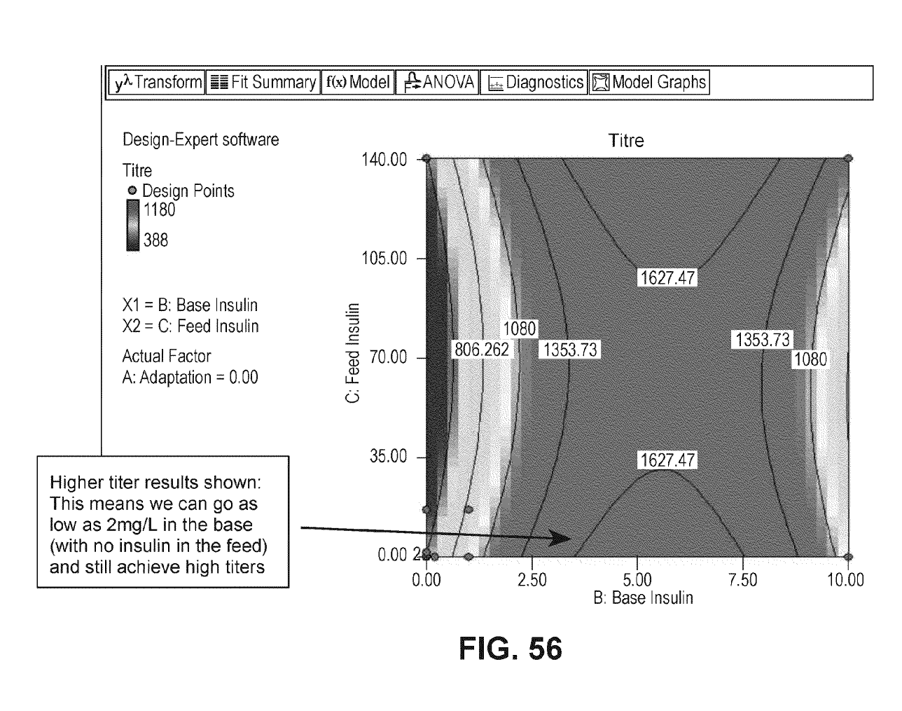

FIGS. 55 and 56 depict exemplary heatmaps produced by analysis using Design-Expert® Software, indicating predicted desired results (e.g., titer in FIG. 56) in cell cultures grown in a range of insulin concentrations in the base medium (B; X axis) and feed medium (C; Y axis). As shown in FIG. 56, highest titer predictions are indicated in red, while lowest titer predictions are indicated in blue. FIG. 56 illustrates that it may be possible to obtain desirable cell culture results (e.g., high titers) using as little as about 2 mg/L insulin supplemented in the base medium of a fed-batch culture, and no insulin in the feed medium.

### EQUIVALENTS

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any linking agent, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

### INCORPORATION OF REFERENCES

All publications and patent documents cited in this application are incorporated by reference in their entirety to the same extent as if the contents of each individual publication or patent document were incorporated herein.

What is claimed is:

1. A method of cell culture comprising a step of cultivating cells adapted to growth factor-free medium in a cell culture system that provides at least one growth factor.

2. The method of claim 1, wherein the method further comprises a step of first adapting the cells to a growth factor-free medium.

3. The method of claim 2, wherein the adapting step comprises growing the cells in the growth factor-free medium for more than approximately 20 generations, or for approximately 30-300 generations, or for approximately 25-50 generations.

4-5. (canceled)

6. The method of claim 3, wherein the growth factor-free medium is substantially free of insulin, and/or
the growth factor-free medium is substantially free of growth factors; and/or
the growth factor-free medium is substantially free of protein; and/or
the growth factor-free medium is substantially free of insulin, peptone, hydrolysates, transferrins, and insulin-like growth factor I (IGF-I).

7-9. (canceled)

10. The method of claim 3, wherein the growth factor-free medium is serum-free or serum-free, protein-containing medium.

11. (canceled)

12. The method of claim 3, wherein the adapting step comprises first growing the cells in a medium comprising a growth factor before growing the cells in the growth factor-free medium.

13. The method of claim 12, wherein the medium comprising the growth factor is a serum-free medium comprising the growth factor.

14. The method of claim 3, wherein the cell culture system is a fed batch system.

15. The method of claim 14, wherein the fed batch system comprises a base medium supplemented with feed media.

16. The method of claim 15, wherein the at least one growth factor is provided in the base medium or in the feed media.

17. The method of claim 16, wherein the at least one growth factor is provided in the base medium but not in the feed media.

18. (canceled)

19. The method of claim 14, wherein the base medium and/or feed media are otherwise substantially free of other growth factors except the at least one growth factor, or wherein the base medium and/or feed media are otherwise substantially free of peptone, hydrolysates, and/or transferrins except the at least one growth factor, or wherein the base medium and/or feed media are otherwise substantially free of protein except the at least one growth factor, or wherein the base medium and/or feed media are substantially free of serum.

20-22. (canceled)

23. The method of claim 3, wherein the at least one growth factor is selected from the group consisting of insulin, insulin-like growth factor (IGF-I), synthetic IGF-I (LR3) and combination thereof.

24. The method of claim 3, wherein the at least one growth factor is insulin.

25. The method of claim 24, wherein the insulin is provided at a concentration ranging from approximately 0.01 mg/L to 1 g/L, or a concentration of approximately 10 mg/L, or at a concentration of approximately 2 mg/L.

26-27. (canceled)

28. The method of claim 3, wherein the at least one growth factor is LR3.

29. The method of claim 28, wherein the LR3 is provided at a concentration ranging from approximately 1 ng/L to 1 mg/L, or a concentration of approximately 1 ng/L to 100 μg/L, or at a concentration of approximately 5 μg/L, or at a concentration of approximately 50 μg/L.

30-32. (canceled)

33. The method of claim 3, wherein
the cell culture system is a large-scale production system; and/or
the cell culture system uses a bioreactor; and/or
the cell culture system uses a shaken culture system.

34-35. (canceled)

36. The method of claim 3, wherein the cells are mammalian cells.

37. (canceled)

38. The method of claim 3, wherein the cells express a recombinant protein.

39-43. (canceled)

44. The method of claim 3, wherein the cells are cultivated under conditions such that the cell growth and/or productivity are increased as compared to control cells that are not first adapted to growth factor-free medium.

45. The method of claim 3, wherein the cells are cultivated under conditions such that the cell growth and/or productivity are increased as compared to control cells that are cultivated in growth factor-free medium without the at least one growth factor.

46-51. (canceled)

52. A recombinant protein produced using the method of claim 3.

53. A method of cell culture comprising steps of:
adapting cells to insulin-free culture;
cultivating the cells in a medium that comprises insulin or an insulin-like growth factor;
wherein the cells are cultivated under conditions such that the cell growth and/or productivity is increased as compared to control cells that are not first adapted to insulin-free culture but cultivated under otherwise identical conditions.
